(19) United States
(12) Patent Application Publication
(10) Pub. No.: US 2010/0178299 A1
(43) Pub. Date: Jul. 15, 2010

(54) METHODS AND COMPOSITIONS FOR IMPROVING IMMUNE RESPONSES

(75) Inventors: Michail V. Sitkovsky, Boston, MA (US); Akio Ohta, Newton, MA (US); Dmitriy Lukashev, Ashland, MA (US)

Correspondence Address: ROPES & GRAY LLP, PATENT DOCKETING 39/41, ONE INTERNATIONAL PLACE, BOSTON, MA, 02110-2624, US

(73) Assignee: Northeastern University, Boston, MA (US)

(21) Appl. No.: 12/527,181

(22) PCT Filed: Feb. 13, 2008
(86) PCT No.: PCT/US08/01891

§ 371 Date: Mar. 5, 2010

Related U.S. Application Data
(60) Provisional application No. 60/901,135, filed on Feb. 13, 2007.
(60) Provisional application No. 60/965,155, filed on Aug. 17, 2007.

(51) Int. Cl. A61K 39/00; A61P 35/00; C12Q 1/02

(52) U.S. Cl. 424/184.1; 435/29

(57) ABSTRACT

The present invention relates to compositions and methods for enhancing an immune response, for example to a vaccine, by combining the administration of oxygen (O₂ gas), an adenosine pathway antagonist and/or an HIF-1α antagonist, and/or inhibitors of enzymes that produce or generate adenosine with the administration of the vaccine to the patient.

The present invention also relates to methods of inducing or enhancing immune responses, methods of treating subjects having a tumor, methods of ablating or killing tumor cells and methods of disrupting the blood supply to a tumor, comprising administering oxygen alone or in combination with therapeutic agents that prevent inhibition of anti-tumor T cells. Tumor defense-resistant immune cells, anti-viral immune cells, and methods of their production are also disclosed.

### RELATED APPLICATIONS

This application claims the benefit of U.S. Provisional Application No. 60/901,135, filed Feb. 13, 2007, and U.S. Provisional Application No. 60/965,155, filed Aug. 17, 2007, the specifications of which are hereby incorporated herein by reference in their entirety.

### GOVERNMENT SUPPORT

The invention described herein was supported, in whole or in part, by grants 1 R01 CA1112561-01, 1 R21AT002788-01 A1, and 1 R01 CA111985-01 A2 from the U.S. National Institutes of Health. The United States Government has certain rights in the invention.

### BACKGROUND OF THE INVENTION

Cancer is one of the deadliest illnesses in the United States. It accounts for nearly 600,000 deaths annually, and costs billions of dollars for those who suffer from the disease. This disease is in fact a diverse group of disorders, which can originate in almost any tissue of the body. In addition, cancers may be generated by multiple mechanisms including pathogenic infections, mutations, and environmental insults (see, e.g., Pratt et al., Hum. Pathol. 36:861-70, 2005). The variety of cancer types and mechanisms of tumorigenesis add to the difficulty associated with treating a tumor, increasing the risk posed by the cancer to the patient's life and wellbeing.

Current cancer treatments include, among others, surgery, chemotherapeutics, radiation therapy, immunotherapy, and photodynamic therapy. However, none of these treatments is completely effective, and each has its own associated side effects. Further, hypoxia is a characteristic feature of locally advanced solid cancers resulting from an imbalance between oxygen supply and consumption (see Vaupel et al., Oncologist 9 Suppl. 5:4-9, 2004). Cancer tumor hypoxia can reduce the effectiveness of radiotherapy, some oxygen-dependent cytotoxic agents, and photodynamic therapy.

Invasive tumor growth and metastasis require angiogenesis, a physiological process involving the growth of new blood vessels and improved delivery of oxygen to oxygen-starving tumors. It is considered advantageous to prevent oxygen and nutrient delivery to tumors, and numerous studies have evaluated the use of angiogenesis inhibitors to suppress tumor growth (see, e.g., Folkman, Semin. Oncol. 29:15-18, 2002). Such inhibitors cut off the supply of oxygen to tumors, starving the tumors of oxygen and leading to apoptosis. The first angiogenesis inhibitors for cancer have now been approved by the FDA in the U.S. and in 28 other countries. The majority of these are monotherapies that block VEGF (see, e.g., Folkman, Exp. Cell. Res. 312:594-607, 2006). However, these drugs can have side effects, such as increasing the risk of internal bleeding, increasing the risk of developing a hole in the digestive tract, and raising blood pressure. Further, cancer tumor hypoxia can induce changes in the proteome and genome of neoplastic cells that further survival and malignant progression by enabling the cells to overcome nutritive deprivation or to escape their hostile environment.

Certain cancers, as well as other disorders such as asthma, emphysema, AIDS, arthritis, heart and vascular diseases, multiple sclerosis, Alzheimer's disease, scarlet fever, diphtheria, and pneumonia, have been alternatively treated with ozone gas and hydrogen peroxide. However, hydrogen peroxide has been known to be toxic if administered in high doses, and, according to the FDA, ozone is a toxic gas with no known useful medical application in specific, adjunctive, or preventive therapy. Long term oxygen therapy has been used in patients with chronic hypoxemia that can occur in several respiratory and cardiac disorders, including chronic obstructive pulmonary disease (COPD), chronic severe asthma and interstitial lung diseases. In patients with hypoxemia, oxygen supplementation improves survival, pulmonary hemodynamics, exercise capacity and improves the quality of life. On the other hand, the toxic consequences to the lung of prolonged exposure to high oxygen tension are known (see, e.g., Jenkinson, New Horiz. 1:504-511, 1993).

Thus, there still remains a need for treatments for cancer as well as other diseases, such as asthma, emphysema, AIDS, arthritis, heart and vascular diseases, multiple sclerosis, Alzheimer's disease, scarlet fever, diphtheria, and pneumonia, that are effective and nontoxic.

Cancer vaccines have been a subject of much attention. Various kinds of cancer vaccines, including tumor vaccines have been developed (Pardoll, D. M., Nature Med., 4(5 Suppl), pp. 525-531, 1998). Roughly tumor vaccines can be categorized depending on tumor-specific materials as follows: (1) vaccines wherein a tumor antigenic peptide with a known property is used; (2) vaccines wherein a tumor tissue extract containing an unidentified tumor antigenic peptide is used; (3) vaccines wherein the above peptide is bound to an antigen-presenting cell, especially a dendritic cell with a strong capability of antigen presentation (Nestle et al., Nature Med., 4, pp. 328-332, 1998); (4) vaccines wherein a tumor antigenic protein is taken into a dendritic cell and loaded; (5) vaccines wherein a dendritic cell and a tumor cell are fused; (6) vaccines wherein a tumor antigen is bound to a liposome for uptake together with the liposome (Nakanishi et al., Biochem. Biophys. Res. Comm., 240, pp. 793-797, 1997); (7) vaccines wherein a tumor cell, per se, is treated for inactivation with radiation or a fixing agent before administration; (8) vaccines wherein a cytokine gene, having an antigen-presenting cell stimulating effect or a lymphocyte stimulating effect, is introduced into a tumor cell and the cell is administered as a vaccine for a gene therapy, or wherein a tumor antigenic gene is introduced into a suitable cell and a tumor cell expressing the gene is administered as a vaccine; (9) vaccines wherein a tumor antigenic gene is integrated into a virus or a bacterium for infection of a patient; (10) vaccines wherein a live tumor cell, a tumor antigenic peptide or an extract of a tumor cell is administered, and separately a great amount of a cytokine is administered (Rosenberg et al., Nature Med., 4, pp. 321-327, 1998), or wherein a cytokine is formulated into a controlled release preparation and administered (Golumbek, P. T., et al., Cancer Res., 53, pp. 5841-5844, 1993) and the like.

Traditionally, the immunogenicity of a vaccine formulation has been improved by injecting it in a formulation that includes an adjuvant. Immunological adjuvants were initially described by Ramon (1924, Ann. Inst. Pasteur, 38: 1) “as substances used in combination with a specific antigen that produced a more robust immune response than the antigen alone.” A wide variety of substances, both biological and synthetic, have been used as adjuvants. However, despite extensive evaluation of a large number of candidates over many years, the only adjuvants currently approved by the U.S. Food and Drug administration are aluminum-based minerals including aluminum compounds (generically called Alum). Alum has a debatable safety record (see, e.g., Malakoff, Science, 2000, 288: 1323), and comparative studies show that it is a weak adjuvant for antibody induction to protein subunits and a poor adjuvant for Cell Mediated Immune responses. Moreover, Alum adjuvants can induce IgE antibody response and have been associated with allergic reactions in some subjects (see, e.g., Gupta et al., 1998, Drug Deliv. Rev. 32: 155-72; Relyveld et al., 1998, Vaccine 16: 1016-23). Many experimental adjuvants have advanced to clinical trials since the development of Alum, and some have demonstrated high potency but have proven too toxic for therapeutic use in humans. Thus, an on-going need exists for safe and potent immunostimulatory agents. As used herein, an immunostimulatory agent refers to an agent that stimulates, enhances, or potentiates a desired immune response. This immune response may be, for example, greater CD4+ cell anti-tumor activity, or greater production of a specific immunoglobulin.

Furthermore, many adjuvants are administered prior or simultaneous to immunization, in order to prime the immune system. There is a shortage of immunostimulatory agents that are effective when administered after the immunization.

Frequently, purified antigens from parasites, bacterial or viral pathogens, as well as recombinant subunit antigens and synthetic peptides, are inherently weak immunogens. Thus, it is important to combine the antigen with an adjuvant or other immunostimulatory agents to trigger stronger immune responses

In addition, recently, there appears to be an emerging consensus that tumor vaccines are less likely to be successful in the context of high tumor burden/load (see, e.g., Nature Medicine Commentary, 10(12): 1278 (2004) and Cancer Immunol. Immunother., 53(10): 844-54 (2004)). This is attributed to effective tumor-mediated immune suppression due to the secretion of IL-10, TGF-β, and PGE-2, among others that may suppress anti-tumor T cells responses.

Therefore, there is an unmet demand for new and effective vaccine adjuvants as well as immunostimulatory agents.

### SUMMARY OF THE INVENTION

The present invention relates to pharmaceutical compositions that are useful for the prevention and treatment of infectious diseases, primary and metastatic neoplastic diseases (i.e., cancer), neurodegenerative or amyloid diseases, or any other disease wherein the treatment of such disease would be improved by an enhanced immune response, and methods of formulating the compositions.

The present invention also relates to methods of using the compositions of the invention for treatment of patients. It is understood that the methods and compositions of the invention enhance the immune response to vaccines by preventing or reducing the physiological down-regulation of immune response in normal, inflamed or cancerous tissues resulting from, for example, secretion of adenosine and/or hypoxic conditions.

One aspect of the present invention relates to methods and compositions for eliciting an enhanced immune response from an immunogen in a patient. The method can be generally characterized as including a step of administering to the subject (human or veterinary patient) one or more of oxygen (e.g., O₂ gas) or an agent that enhances oxygen delivery to peripheral tissues, an adenosine pathway antagonist or a HIF-1α antagonist in conjunction with administering the immunogen, such as in the form of a vaccine, to the patient.

Another aspect of the present invention relates to vaccine formulations for eliciting an enhanced immune response to an immunogen. The subject vaccines include the immunogen along with an adenosine pathway antagonist (such as an adenosine receptor antagonist) and/or an HIF-1α antagonist.

In one embodiment, the present disclosure provides a method of treating cancer, comprising conjointly administering a therapeutically effective amount of an A2AR antagonist and a cancer vaccine to a patient in need thereof. The present disclosure also provides a method of treating cancer, comprising conjointly administering a therapeutically effective amount of an A2AR antagonist and oxygen to a patient in need thereof. Also disclosed is a method of treating solid tumors, comprising conjointly administering a therapeutically effective amount of an A2AR antagonist together with a vasculature-targeting agent to a patient in need thereof. This application also provides a method of treating cancer, comprising conjointly administering a therapeutically effective amount of at least 45% or 50% oxygen to a patient in need thereof. Also disclosed is a method of vaccinating an individual, comprising conjointly administering an effective amount of a pathogen vaccine and an effective amount of an A2AR antagonist to the individual. The present application discloses, inter alia, a method of eliciting an enhanced immune response to a cancer cell, comprising conjointly administering a therapeutically effective amount of an A2AR antagonist and a cancer vaccine to a patient in need thereof. Also disclosed is a method of eliciting an enhanced immune response to a cancer cell, comprising conjointly administering a therapeutically effective amount of an A2AR antagonist and oxygen to a patient in need thereof.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

The disclosed methods may include a first (priming) immunization and at least one subsequent booster immunization, and said A2AR antagonist is administered conjointly with at least one booster immunization. The vaccine may be administered repeatedly or continuously. When the cancer vaccine is for a solid tumor, the 2AR antagonist may be delivered locally to the site of the tumor. The A2AR antagonist may be administered repeatedly or continuously. For example, the A2AR antagonist may be administered repeatedly or continuously for a period of at least 1, 2, 3, or 4 weeks; 2, 3, 4, 5, 6, 8, 10, or 12 months; or 2, 3, 4, or 5 years.

Furthermore, the A2AR antagonist may be administered continuously or periodically between the priming and booster immunization. In certain embodiments, the vaccine is administered simultaneously with the A2AR antagonist. Alternatively, the A2AR antagonist may be administered before the vaccine. However, in a preferred embodiment, the A2AR antagonist is administered after the vaccine.

The A2AR antagonist may be administered 1, 2, 3, 5, 7, or more days after the vaccine. In another aspect, the A2AR antagonist may be administered 2, 3, 4, 5, or 6 or more weeks after the vaccine. Alternatively, the A2AR antagonist may be administered after a certain biological event. For example, the A2AR antagonist may be administered after antigen presenting cells present the vaccine antigen. Alternatively, the A2AR agonist may be administered after helper T cells activate B cells specific to the vaccine antigen. In other embodiments, the A2AR agonist may be administered after vaccine antigen-specific B cells exhibit class switching; after vaccine antigen-specific T cells undergo T cell expansion, and after memory T cells specific to the vaccine antigen are produced. In an especially preferred invention, the A2AR antagonist is administered after expansion of T cells specific to the vaccine. Alternatively, the A2AR antagonist is administered after the differentiation of CD4+ helper T cells, regulatory T cells (Treg cells) or both, specific to the vaccine antigen. In yet another embodiment, the A2AR antagonist is administered at a time when the vaccine is present at an effective serum concentration.

In the claimed methods, the cancer may be one of a variety of cancers including melanoma, prostate cancer, breast cancer, ovarian cancer, esophageal cancer, or kidney cancer. The cancer may be a solid tumor, blood cancer, or lymphatic cancer. The cancer may be benign or metastatic.

The methods herein may be practiced wherein oxygen is administered to the patient. The oxygen may be supplemental oxygen. Oxygen may be administered at different levels including 21%, 25%, 30%, 40%, 45%, 50%, 60%, 70%, 80%, 90%, and essentially 100%. The oxygen may be administered to the patient simultaneously with the A2AR antagonist. The oxygen may be administered using an oxygen delivery device, examples of which are provided herein.

The methods described herein may further be practiced by administering at least one additional anti-cancer therapy to the patient, wherein the additional anti-cancer therapy is selected from the group consisting of radiation therapy, chemotherapy, surgery, vasculature-targeting therapy, and a cancer vaccine. The cancer vaccine may be, for example, a melanoma vaccine. Vasculature-targeting therapy is defined herein as the administration of a vasculature-targeting agent.

In keeping with the present disclosures, one or more biomarkers may be used to assay the status of the cancer. Exemplary biomarker are CA-125, CA-19-9, or PSA, and more are listed throughout the specification.

The A2AR antagonist may be any A2AR antagonist. Specifically, the A2AR may be one of the following: caffeine and/or a caffeine derivatives; (−)-(R,S)-mefloquine; 3,7-Dimethyl-1-propargylxanthine (DMPX); 3-(3-hydroxypropyl)-7-methyl-8-(m-methoxystyryl)-1-propargylxanthine (MSX-2); 3-(3-hydroxypropyl)-8-(3-methoxystyryl)-7-methyl-1-propargylxanthine phosphate disodium salt; 7-methyl-8-styrylxanthine derivatives; 7-(2-phenylethyl)-5-amino-2-(2-furyl)-pyrazolo-[4,3-ε]-1,2,4-triazolo[1,5c]pyrimidine (SCH 58261); (E)-1,3-diethyl-8-(3,4-dimethoxystyryl)-7-methyl-3,7-dihydro-1H-purine-2,6-dione (Istradefylline, or KW-6002); aminofuryltriazolo-triazinylaminoethylphenol (ZM 241385); 8-chlorostyrylcaffeine; (E)-1,3-dipropyl-8-(3,4-dimethoxystyryl)-7-methyl-3,7-dihydro-1H-purine-2,6-dione (KF17837); 2-isopropyl-4-(2-thiazolyl)thieno[3,2-d]pyrimidine-2-amine (VR2006); the VERNALIS drugs such as VER 6489, VER 6623, VER 6947, VER 7130, VER 7146, VER 7448, VER 7835, VER 8177, VER 11135, VER 6409, VER 6440; pyrazolo[4,3-e]1,2,4-triazolo[1,5-c]pyrimidines; and 5-amino-imidazolo-[4,3-e]-1,2,4-triazolo[1,5-c]pyrimidines.

In a preferred embodiment, the A2AR antagonist is selective for A2AR. For instance, the Kᵢ of the A2AR antagonist for A2AR may be at least 10-fold lower than the Kᵢ of said antagonist for A1R. Additionally, the Kᵢ of the A2AR antagonist for A2AR may be at least 10-fold lower than the Kᵢ of said antagonist for A2BR. Furthermore, the Kᵢ of the A2AR antagonist for A2AR may be at least 10-fold lower than the Kᵢ of said antagonist for A3R. Also, the Kᵢ of the A2AR antagonist for A2AR may be at least 10-fold lower than the Kᵢ of said antagonist for one or more AMP, ADP, or ATP receptors. In other embodiments, the Kᵢ of the A2AR antagonist for A2AR may be at least 20-fold, 50-fold, 100-fold, 200-fold, 500-fold, 1000-fold, 2000-fold, 5000-fold, or 10000-fold lower than the Kᵢ of said antagonist for A1R, A2BR, A3R, or one or more AMP, ADP, or ATP receptors.

In the claimed methods, an adjuvant may also be administered. The adjuvant may be, for example, Alum. Other adjuvants include FLT3 ligand and IL2. An adjuvant may be a substance that increases the numbers or activity of antigen presenting cells such as dendritic cells. QS-21 Stimulon, for example, may be used as an adjuvant.

In any of the methods described herein, oxygen may be administered using a mask, intubation, mechanical ventilation, or a hyperbaric chamber. In certain embodiments, the oxygen is administered while the patient sleeps. In certain embodiments, the oxygen is administered at night.

The vasculature-targeting agents of the disclosed methods may inhibit vascular neogenesis (that is, inhibit the growth of new blood vessels), impair the function of pre-existing vasculature, normalize tumor vasculature or perform two or more of these functions. In certain embodiments, the vasculature-targeting agent is thalidomide, combretastatin, taxol, STI571, C225, Herceptin, or angiostatin. The A2AR antagonist may be administered concurrently with the vasculature-targeting agent. Alternatively, the A2AR antagonist may be administered after the vasculature-targeting agent.

In certain embodiments, the patient being treated is immunocompromised. In certain embodiments, the patient is HIV positive (infected with human immunodeficiency virus); in certain embodiments the patient is suffering from AIDS. In other embodiments, the patient is receiving or has received chemotherapy. The subject may be receiving immunosuppressive therapy.

In certain aspects, the vaccine is weakly immunogenic. In some aspects, the vaccine is an HIV vaccine.

The methods herein also provide a method of vaccinating an individual, comprising: (a) administering a therapeutically effective amount of a vaccine to an individual, (b) determining the level of a biomarker in the individual, (c) determining whether the level of the biomarker is significantly different from a control level, and (d) only administering an A2AR antagonist to the patient if the biomarker level is significantly different from the control level. In some embodiments, the level of the biomarker is greater than the level of the control. Alternatively, the level of the biomarker may be less than the level of the control. The vaccine may be, for example, a cancer vaccine or a pathogen vaccine. In certain embodiments, the biomarker correlates with cancer progression. In other embodiments, the biomarker correlates with immune system activity. The biomarker may be a cytokine level, a white blood cell count, or an immunoglobulin level.

The A2AR antagonist may be delivered in a localized dose. For example, the dose may be localized to a solid tumor, to the thyroid, to the bloodstream, and to the lymph system. The dose may be delivered via stereotactic injection. The dose may be delivered via a controlled release drug delivery system. The A2AR antagonist may also be administered in nanoparticles. The nanoparticles may be Nanocell nanoparticles. The antagonist may be covalently linked or noncovalently bound to a targeting moiety such as, for example, an antibody.

The present application also provides a method of enhancing a B cell response of a non-human animal, comprising: (a) administering an immunogen to a non-human animal, and (b) administering an additional therapeutic to the animal. This therapeutic may be, for example, an adenosine pathway antagonist (such as an A2AR antagonist), oxygen (such as supplemental oxygen), or a combination of therapies. Said method may result in increased immunoglobulin levels (for example levels of IgG) in the non-human animal. Preferable, a substantial portion of said immunoglobulin is specific to the immunogen. Oxygen may be administered at different levels including 21%, 25%, 30%, 40%, 45%, 50%, 60%, 70%, 80%, 90%, and essentially 100%. The animal may be, for example, a mouse, chicken, rabbit, guinea pig, goat, donkey, or horse. This method may further comprise drawing blood from the animal and purifying an antibody from the blood. A polyclonal antibody may be prepared using this method. The claimed methods may also further comprising harvesting B cells from the animal. The methods may also include fusing the B cells to a cancer cell such as a myeloma cell to form a hybridoma. The hybridoma may secrete monoclonal antibody. The monoclonal antibody may be purified using any means known in the art, such as affinity for protein A or protein G. In other embodiments, the disclosed methods further comprise conjointly administering an adjuvant to the animal.

The present disclosure also teaches a method of screening for an adenosine receptor antagonist, comprising: (a) contacting an immune cell with an agent; (b) exposing the immune cell to high oxygen levels, and (c) assaying for increased activity of the immune cell as compared to a control in the absence of the agent, wherein increased activity of the immune cell indicates that the agent is an adenosine receptor antagonist. The immune cell may be a cell that produces at least one inflammatory cytokine. The immune cell may be a macrophage, granulocyte, monocyte, neutrophil, dendritic cell, T cell, B cell, or natural killer cell. The increased activity may comprise increased cAMP, increased cytokines, increased apoptosis, and/or morphological changes. In the disclosed methods, the subject may have one or more of smallpox, yellow fever, distemper, cholera, fowl pox, scarlet fever, diphtheria, tetanus, whooping cough, influenza, rabies, mumps, measles, foot and mouth disease, or poliomyelitis.

The disclosures herein also provide a method of inducing or enhancing an immune response in a subject in need thereof, comprising conjointly administering oxygen to the subject in an amount sufficient to induce or enhance the immune response, wherein the oxygen is administered in a hyperbaric chamber or as supplemental oxygen. The oxygen may be administered at a level that is increased relative to the level of ambient oxygen. The oxygen therapy may be administered for between about 1 hour and about a few weeks. The oxygen therapy may be administered once per day. The method may further comprise the step of evaluating the subject for a marker of an induced or enhanced immune response. The immune response may comprise a cell-mediated immune response. This response may comprise the activity of one or more of a macrophage, granulocyte, monocyte, neutrophil, dendritic cell, T cell, B cell, or a natural killer cell. This response may comprise a cell-mediated cytolytic immune response. This response may comprise a humoral immune response, an inflammatory response, a pro-inflammatory cytokine response, including an increase in the expression of one or more of interferon gamma, interferon beta, interferon alpha, IL-12p40, TNF-alpha or IL-17 mRNA relative to the level before oxygen administration. The level of expression of one or more of interferon gamma, interferon beta, interferon alpha, IL-12p40, TNF-alpha or IL-17 mRNA may be evaluated relative to the level before oxygen administration. The disclosed methods may further comprise conjointly administering a therapeutically effective amount of a therapeutic agent to the subject. The oxygen and the therapeutic agent may be administered concurrently or sequentially. In some embodiments, the oxygen is administered prior to the therapeutic agent. In certain embodiments, the therapeutic agent is an oxygen-enhancing substance.

In some embodiments, the therapeutic agent is an A2a or A2b adenosine receptor antagonist. The agonist may be selected from the group consisting of ZM241385, 1,3,7, trimethylxanthine (caffeine), theophilline, teobromin, SCH5826, KW-6002, and ADA-PEG. In other embodiments, the therapeutic agent is an A1 adenosine receptor agonist or an A3 adenosine receptor agonist. The therapeutic agent is an inhibitor of extracellular adenosine, an agent that decreases inflammation-associated local tissue hypoxia, an agent that decreases the redox status of molecules in an inflamed local tissue environment, or an immunostimulant. The therapeutic agent may be an inhibitor of extracellular adenosine selected from the group consisting of an agent that degrades extracellular adenosine in tissues, an agent that increases endogenous adenosine kinase activity, an agent that increases endogenous adenosine deaminase activity, an oxygenation agent, a redox-potential changing agent, an adenosine-accumulation-reducing agent, adenosine deaminase, and adenosine kinase. The therapeutic agent may be an immunostimulant selected from the group consisting of IFA, a COX-2 inhibitor, IL-12, saponin, and N-acetyl-cysteine.

In certain aspects, the subject is infected with a virus, bacterium, or fungus.

The present disclosure also provides a method of treating a subject having a tumor, comprising administering oxygen to the subject in an amount sufficient to reduce tumor size, volume, or number of tumor cells. The oxygen may be administered in a hyperbaric chamber or as supplemental oxygen. The tumor to be treated may be greater than about 0.1, 0.2, 0.5, 1, 2, 5, 10, 20, 50, or 100 mm in diameter. The tumor to be treated may have localized hypoxia areas. The tumor to be treated may be hypoxic throughout. The tumor may be a tumor of the kidney, urinary tract, colon, rectum, lung, liver, breast, prostate or skin.

The methods described herein may further comprise administering a therapeutically effective amount of an anti-tumor agent. The anti-tumor agent may selectively target the cells of the tumor. In certain embodiments, the anti-tumor agent is a nucleic acid molecule that encodes a protein that promotes apoptosis. In some embodiments, the anti-tumor agent is an alkylating drug, a folate antagonist, a purine antagonist, a pyrimidine antagonist, a spindle poison, a podophyllotoxin, an antibiotic, a nitrosurea, an inorganic ion, a biologic response modifier, an enzyme, or a hormone. The methods herein may further comprise one or more of surgery, cryosurgery, radiation therapy, thermotherapy, hormone therapy, chemotherapy, administration of a vaccine, or administration of an antibody. The method may increase efficiency of tumor-infiltrating lymphocytes (TIL). The method may also decrease the immunosuppressive activities of T regulatory cells (Tregs). The methods herein may also include ablating or killing tumor cells, comprising administering oxygen to the tumor cells in an amount sufficient to ablate or kill the tumor cells.

Also disclosed is a method of inducing or enhancing an immune response in a subject, the method comprising: (a) administering to the subject a vaccine which elicits an immune response; and (b) administering oxygen the subject in a hyperbaric chamber or as supplemental oxygen, wherein the oxygen induces or enhances the immune response stimulated by the vaccine. In some embodiments, the vaccine comprises an antigenic polypeptide or an antigenic epitope thereof. The vaccine may be a viral vaccine. The viral vaccine may be a live, attenuated, or heat killed vaccine. The vaccine may induce anti-tumor or anti-pathogen T cells.

The present disclosures also provide a method of producing a tumor defense-resistant immune cell or an anti-viral immune cell, comprising culturing an immune cell under hypoxic culture conditions to produce an immune cell that is resistant to hypoxia-produced extracellular adenosine, thereby producing a tumor defense-resistant immune cell or an anti-viral immune cell. In certain embodiments, the hypoxic culture conditions comprise less than 4% oxygen. In certain embodiments, the immune cell is a cytotoxic T lymphocyte or a lymphokine-activated killer cell. The present disclosure also provides an isolated tumor defense-resistant immune cell or an anti-viral immune cell produced by a method disclosed herein.

Additionally provided is a method of treating a subject having a tumor, comprising administering one or more disclosed cells to the subject, thereby reducing tumor size, tumor volume, and/or number of cells in the tumor. Applicants also disclose a method of enhancing an immune response to a virus in a subject, comprising administering one or more cells described herein to the subject, thereby enhancing the immune response to the virus in the subject. Also disclosed is a method of disrupting the blood supply to a tumor in a subject, comprising administering oxygen to the subject in an amount sufficient to disrupt the blood supply to the tumor, wherein the oxygen is administered in a hyperbaric manner or as supplemental oxygen. The disruption of the blood supply may result in a reduction of tumor volume and/or a reduction in the number of tumor cell in the subject.

In certain embodiments, the present disclosure provides a use of an A2AR antagonist in the manufacture of a medicament for enhancing the response of a patient to a vaccine, for example by enhancing the immune response to a cancer cell. The vaccine may be, for example, a pathogen vaccine or a cancer vaccine. In other embodiments, the present disclosure provides a use of an A2AR antagonist in the manufacture of a medicament for treating cancer, for example by enhancing the immune response to a cancer cell, as part of a therapeutic regimen. This therapeutic regimen may additionally comprise administering oxygen, administering a vasculature-targeting agent, or administering another cancer therapy. Furthermore, the present disclosure provides, inter alia, a use of at least 45% or 50% oxygen in preparing a device (such as an oxygen tank with an oxygen mask) for treating cancer. The amount of oxygen may be at least 40%, 60%, 70%, 80%, 90%, or essentially 100%.

In addition, the present application discloses a use of a kit comprising an A2AR antagonist and a biomarker assay tool. The A2AR antagonist may be administered to the patient, and the biomarker assay tool may be used to gauge the effectiveness of the A2AR antagonist. Alternatively, the biomarker assay tool may be used to assay the state of the patient in order to determine whether to administer the A2AR antagonist, and the amount, frequency and duration of A2AR antagonist administration.

In certain embodiments, the present disclosure provides a pharmaceutical preparation comprising an A2AR antagonist and another therapeutic. This therapeutic may be a vaccine (for example, a cancer vaccine or pathogen), supplemental oxygen, or an anti-cancer therapeutic (such as a chemotherapeutic agent or a vasculature-targeting agent).

In certain embodiments, the instant disclosure teaches a kit comprising an A2AR antagonist and another component. The other component may be, for example, a biomarker assay tool, an oxygen delivery device, or an additional therapeutic agent. The additional therapeutic agent may be, for example, a vaccine (such as cancer vaccine or pathogen vaccine) or an anti-cancer therapeutic agent (such as a vasculature-targeting agent or chemotherapeutic drug).

The present disclosure also provides, for example, use of an A2AR antagonist in the manufacture of a medicament formulated to be administered as a localized dose. For example, the A2AR antagonist may be formulated as nanoparticles or may be fused with a targeting moeity.

In other aspects, the present disclosure provides a use of an A2AR antagonist in the manufacture of a medicament formulated for administration to a non-human animal. The A2AR may be formulated to enhance a B cell response of a non-human animal. It may be formulated for co-administration with an immunogen.

Still another aspect of the present invention relates to kits for vaccination to produce an enhanced immune response to an immunogen. Such kits can include:
(i) a vaccine formulation of the immunogen; and
(ii) an adenosine pathway antagonist and/or an HIF-1α antagonist, formulated for administration in conjunction with the vaccine.

In certain embodiments, the vaccine is a tumor vaccine. In other embodiments, the vaccine is a pathogen vaccine.

In another aspect, the present invention teaches that administration of oxygen in combination with an adenosine receptor antagonist increases immune-mediated tumor destruction and increases survival rate in mice having tumors. Accordingly, another aspect of the invention relates to methods of inducing or enhancing immune responses, of treating subjects having a tumor, of ablating or killing tumor cells, of disrupting the blood supply to a tumor, tumor defense-resistant immune cells and methods of their production, and anti-viral immune cells and methods of their production.

In certain embodiments utilizing an adenosine pathway antagonist, it can be an adenosine receptor antagonist. Exemplary adenosine receptor antagonist include those selected from pharmacological agents that impair receptor function, small molecules and antibodies that block the receptor, peptides or proteins that block or inhibit the receptor, small interfering RNA molecules that impair or inhibit transcription of a gene encoding the adenosine receptor, anti-sense RNA that impairs or inhibits the transcription of a gene encoding the adenosine receptor, agents that lead to inhibition, down-regulation, or interference with adenosine receptor activity, and ribozymes with a complementary base pair binding portion that binds to adenosine receptor mRNA and a catalytic portion that cleaves said mRNA. In certain embodiments, the adenosine receptor antagonist is an adenosine A2A receptor antagonists, i.e., at least 2 fold more selective for A2A than other adenosine receptor subtypes and isoforms, and more preferably at least 5, 10 or even 100 fold more selective.

To further illustrate, the adenosine receptor antagonist can be selected from the group consisting of caffeine and/or a caffeine derivatives, (−)-(R,S)-mefloquine (the active enantiomer of the racemic mixture marketed as Mefloquine™), 3,7-Dimethyl-1-propargylxanthine (DMPX), 3-(3-hydroxypropyl)-7-methyl-8-(m-methoxystyryl)-1-propargylxanthine (MX2), 3-(3-hydroxypropyl)-8-(3-methoxystyryl)-7-methyl-1-propargylxanthin phosphate disodium salt (MSX-3, a phosphate prodrug of MSX-2), 7-methyl-8-styrylxanthine derivatives, SCH 58261, SCH 58621, KW-6002, aminofuryltriazolo-triazinylaminoethylphenol (ZM 241385), and 8-chlorostyrylcaffeine, KF17837, VR2006, istradefylline, the VERNALIS drugs such as VER 6489, VER 6623, VER 6947, VER 7130, VER 7146, VER 7448, VER 7835, VER 8177VER-11135, VER-6409, VER 6440, VER 6489, VER 6623, VER 6947, VER 7130, VER 7146, VER 7448, VER 7835, VER 8177, pyrazolo[4,3-e]1,2,4-triazolo[1,5-c]pyrimidines, and 5-amino-imidazolo-[4,3-e]-1,2,4-triazolo[1,5-c]pyrimidines.

In other embodiments utilizing an adenosine pathway antagonist, it can be cAMP antagonist. Exemplary cAMP antagonists include PKA inhibitors and adenylate cyclase inhibitors.

In certain embodiments, the adenosine pathway antagonist is an A2A receptor antagonist (also called an A2AR antagonist).

The adenosine pathway antagonist may be administered at different times. For example, an adenosine receptor antagonist may be administered once. Alternatively, an adenosine receptor antagonist may be administered continuously, for example using a controlled release drug delivery system or an IV drip. In yet other embodiments, the adenosine pathway antagonist may be administered repeatedly. Continuous or repeated administration may take place over the course of, for example, 1, 2, 4, 6, 12, 24, 36, 48, or 60 months.

In certain embodiments utilizing an HIF-1α antagonist, the agent can be selected from the group consisting of cardiac glycosides, PI3 kinase inhibitors; LY294002; rapamycin; histone deacetylase inhibitors; heat shock protein 90 (Hsp90) inhibitors; genistein; indanone; staurosporin; protein kinase-1 (MEK 1) inhibitors; PX-12 (1-methylpropyl 2 imidazolyl disulfide); PX-478 (S-2-amino-3-[4′-N,N,-bis(2-chloroethyl)amino]phenyl propionic acid N-oxide dihydrochloride); quinoxaline 1,4-dioxides; sodium butyrate (NaB); sodium nitropurruside (SNP) and other NO donors; microtubule inhibitors; coumarins, barbituric and thiobarbituric acid analogs; camptothecins; and YC-1.

The immunogens/vaccine formulations used in the present invention can also include additional adjuvants, such as saponins as an example.

Exemplary tumor-associated antigens that can be used in the subject methods and vaccines include such tumor-associated antigen as may be selected from the group of Melan A, MART-1, MAGE-1, MAGE-3, BAGE, GAGE-1, GAGE-2, tyrosinase, gp100, gp75, HER-2/neu, c-erb-B2, CEA, PSA, MUC-1, CA-125, Stn, TAG-72, KSA (17-1A), PSMA, p53, RAS, EGF-R, VEGF, GD2, GM2, GD3, Anti-Id, CD20, CD19, CD22, CD36, Aberrant class II, B1, CD25, or BPV. However, any cancer vaccine may be used in concert with the methods disclosed herein.

In certain embodiments, viral antigens that can be used in the subject methods and vaccines, and can include such viral antigens as those viral antigen elicit an immune response for treating a viral disease selected from the group consisting of viral meningitis, tuberculosis, encephalitis, dengue or smallpox, or it can be an antigen of a virus selected from the group consisting of smallpox virus, hepatitis type A, hepatitis type B, hepatitis type C, influenza, varicella, adenovirus, herpes simplex type I (HSV-I), herpes simplex type U (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, human immunodeficiency virus (HIV), human immunodeficiency virus type I (HIV-I), human immunodeficiency virus type II (HIV-II), rabies virus, and Epstein Barr virus. In certain embodiments, the HIV vaccine comprises the GPI antigen or a portion or mutant thereof.

In still other embodiments, bacterial antigens that can be used in the subject methods and vaccines, such as antigens associated with a bacterium selected from the group consisting of Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcus faecalis, Neisseria meningitidis, Neisseria gonorrhoeae, Bacillus anthracis, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospria spp., Hemophilus ducreyi, Corynebacterium diphtheria, Bordetella pertussis, Bordetella parapertussis, Bordetella bronchiseptica, hemophilus influenza, Escherichia coli, Shigella spp., Erlichia spp., Rickettsia spp. and combinations thereof.

In yet other embodiments, protozoal antigens that can be used in the subject methods and vaccines, such as antigens of a protozoan selected from the group consisting of leishmania, kokzidioa, and trypanosoma.

In one embodiment, the present disclosure provides a method for enhancing treatment of a cancer patient, comprising administering the patient one or more of oxygen, an adenosine pathway antagonist or a HIF-1α antagonist, in conjunction with radiation therapy, ultrasound ablation, thermal ablation, electrical ablation, surgical excision, cryotherapy, laser therapy, phototherapy and the like. Herein are also disclosed combined therapy and vaccination methods to improve an enhanced immune response from an immunogen by step-wise and biomarkers-informed cumulative and escalated disengagement of individual sequential stages of immune response-inhibiting hypoxia-adenosinergic pathway.

In yet another aspect, the invention features a method of inducing or enhancing an immune response in a subject in need thereof, comprising administering oxygen to the subject in an amount sufficient to induce or enhance the immune response, wherein the oxygen is administered in a hyperbaric chamber or as supplemental oxygen.

In one embodiment, 100% oxygen is administered in a hyperbaric chamber. In certain embodiments, the hyperbaric chamber has an internal pressure that is greater than atmospheric pressure at sea level. In particular embodiments, the internal pressure is about 1.5 times greater than, about 2 times greater than, about 2.5 times greater than, about 3 times greater than, about 3.5 times greater than, about 4 times greater than, or more than about 4 times greater than atmospheric pressure at sea level. In some embodiments, the hyperbaric chamber internal pressure results in an arterial oxygen tension in excess of 1000 mm Hg, in excess of 1500 mm Hg, in excess of 2000 mm Hg, in excess of 2500 mm Hg, or in excess of 3000 mm Hg. In other embodiments, the hyperbaric chamber internal pressure results in an oxygen tension in tissue of about 300 mm Hg, of about 350 mm Hg, of about 400 mm Hg, of about 450 mm Hg, or of about 500 mm Hg.

In one embodiment, the oxygen is administered as supplemental oxygen at a level that is increased relative to the level of ambient oxygen. In some embodiments, the oxygen is administered in a gas mixture that includes oxygen at a level between about 10% and about 100%, between about 20% and about 100%, between about 21% and about 100%, between about 25% and about 100%, between about 30% and about 90%, or between about 40% and about 60%. In certain embodiments, the oxygen is administered at a level that is greater than 21%, greater than 30%, greater than 40%, greater than 45%, greater than 50%, greater than 60%, greater than 70%, greater than 80%, greater than 90%, or greater than 95% oxygen. In one particular embodiment, about 60% oxygen is administered to the subject. In another particular embodiment, about 100% oxygen is administered to the subject.

In some embodiments, the supplemental oxygen is supplied by way of a nasal cannula, a nasal catheter or a transtracheal catheter. In other embodiments, the supplemental oxygen is supplied in a sealed chamber with an internal pressure that is not greater than atmospheric pressure at sea level.

In some embodiments, the oxygen is administered for about 1 hr. to about 4 weeks. In certain embodiments, the oxygen is administered for about 1 hr., for about 1.5 hr., for about 2 hr., for about 3 hr., for about 4 hr., for about 6 hr., for about 8 hr., for about 10 hr., for about 12 hr., for about 24 hr., for about 2 days, for about 4 days, for about 1 week, for about 2 weeks, for about 3 weeks, for about 4 weeks, for about 1 month, for about 2 months, for about 6 months, or for more than 6 months.

In certain embodiments, the oxygen is administered at least once per day. In certain embodiments, the oxygen is administered at least once every hr., at least every 2 hr., at least every 4 hr., at least every 8 hr., at least every 12 hr., at least every 24 hr., at least every day, at least every 2 days, at least every 4 days, at least every week, at least every 2 weeks, at least every 4 weeks, at least every month, at least every 2 months, at least every 4 months, at least every 6 months, or more than 6 months.

In certain embodiments, the oxygen provided is present in a mix of gasses having at least 21%, 25%, 30%, 40%, 45%, 50%, 60%, 70%, 80%, 90%, or essentially 100% oxygen. In certain embodiments, the oxygen is delivered to the patient through a mask that does not require intubation. In certain embodiments, the oxygen is delivered to the patient through a mask that does not require ventilation.

In some embodiments, the immune response to be induced or enhanced comprises a cell-mediated immune response. In particular embodiments, the cell-mediated immune response comprises the activity of one or more of a macrophage, granulocyte, monocyte, neutrophil, dendritic cell, T cell, B cell, or a natural killer cell. In certain embodiments, the cell-mediated immune response comprises a cell-mediated cytolytic immune response. In some embodiments, the immune response comprises a humoral immune response. In some embodiments, the immune response comprises an inflammatory response. In certain embodiments, the immune response is a pro-inflammatory cytokine response. In particular embodiments, the pro-inflammatory cytokine response comprises an increase in the expression of one or more of interferon gamma, interferon beta, interferon alpha, IL-12p40, TNF-alpha or IL-17 mRNA, relative to the level before oxygen administration.

In some embodiments, the subject is a vertebrate. In certain embodiments, the subject is a mammal. In particular embodiments, the subject is a human.

In some embodiments, the subject is immunocompromised. In certain embodiments, the subject is infected with human immunodeficiency virus (HIV). In other embodiments, the subject is receiving immunosuppressive therapy such as, for example, chemotherapy or radiation therapy. In certain embodiments, the immunocompromised patient suffers from an inherited immunodeficiency such as SCID. In certain embodiments, the subject is infected with a virus, bacterium, or fungus. In certain embodiments, the subject has or is suffering from one or more symptoms of smallpox, yellow fever, distemper, cholera, fowl pox, scarlet fever, diphtheria, tetanus, whooping cough, influenza, rabies, mumps, measles, foot and mouth disease, or poliomyelitis.

In other embodiments, the method further comprises the step of evaluating the subject for a marker of an induced or enhanced immune response. In certain embodiments, the method comprises evaluating the level of expression of immunoglobulin, cytokines, interferon gamma, interferon beta, interferon alpha, IL-12p40, TNF-alpha, or IL-17 mRNA, relative to the level before oxygen administration. In some embodiments, the subject is evaluated before, during, and/or after oxygen administration. In some embodiments, the oxygen is administered until a predetermined level of an immune response is achieved.

In other embodiments, the method further comprises administering a therapeutically effective amount of a therapeutic agent to the subject.

In certain embodiments, the therapeutic agent is an oxygen-enhancing substance that increases local oxygen tension in the subject. In particular embodiments, the therapeutic agent is an A2a and/or A2b adenosine receptor antagonist. In certain embodiments, the therapeutic agent is ZM241385, 1,3,7, trimethylxanthine (caffeine), theophilline, teobromin, SCH5826, or KW-6002.

In another embodiment, the therapeutic agent is a Gi-coupled adenosine receptor agonist. In certain embodiments, the therapeutic agent is an A1 adenosine receptor agonist or an A3 adenosine receptor agonist.

In yet other embodiments, the therapeutic agent is an inhibitor of extracellular adenosine. In certain embodiments, the inhibitor is an agent that degrades extracellular adenosine in tissues, an agent that increases endogenous adenosine kinase activity, an agent that increases endogenous adenosine deaminase activity, an oxygenation agent, a redox-potential changing agent, an adenosine-accumulation-reducing agent, adenosine deaminase (ADA), or adenosine kinase. In one embodiment, the therapeutic agent is ADA-PEG. In one embodiment, the therapeutic agent is recombinant adenosine deaminase or recombinant adenosine kinase. An additional activator of adenosine kinase is 4-[5-(4-phenoxyphenyl)-2H-pyrazol-3-yl]-morpholine (CD12001). The therapeutic agent may also be potassium, which activates adenosine deaminase. In alternative embodiments, the therapeutic agent is an inhibitor of an adenosine-generating enzyme. For instance, the therapeutic agent may be an inhibitor of CD39 (which is the ATPase/ADPase that generates AMP from ATP and ADP) or CD73 (which is a 5′-Nucleotidase that generates adenosine from AMP). Known inhibitors of CD39 include polyunsaturated fatty acids, as well as azide (although a non-toxic equivalent of azide would be necessary for administration to humans). Inhibitors of CD73 include β-methylene ADP, APCP (available from Sigma-Aldrich), and α,β methylene adenosine 5′-diphosphate (AOPCP). In addition, the therapeutic agent may be an activator of equilibrative nucleoside transporters 1 (ENT1), the membrane transporter that removes adenosine from the extracellular space.

In certain embodiments, the present disclosure provides a method of enhancing the immune response of a patient, comprising conjointly administering a therapeutically effective dose of an A2AR antagonist and an inhibitor of an adenosine-producing enzyme. The adenosine-producing enzyme may be, for example, CD39 (Ectonucleoside triphosphate diphosphohydrolase 1) or CD73 (Ecto-5′-nucleotidase).

In some embodiments, the therapeutic agent is an agent that decreases inflammation-associated local tissue hypoxia or decreases the redox status of molecules in an inflamed local tissue environment. In particular embodiments, the therapeutic agent is an immunostimulant. In certain embodiments, the immunostimulant is IFA, a COX-2 inhibitor, IL-12, saponin, or N-acetyl-cysteine.

In some embodiments, the oxygen is administered in combination with one or more therapeutic agents. In certain embodiments, the oxygen is administered in combination with (i) an A2a adenosine receptor antagonist or A2b adenosine receptor antagonist, and (ii) an A1 adenosine receptor agonist or an A3 adenosine receptor agonist.

In some embodiments, the oxygen and the therapeutic agent are administered concurrently. In other embodiments, the oxygen and the therapeutic agent are administered sequentially. In certain embodiments, the oxygen is administered prior to the therapeutic agent. In other embodiments, the oxygen is administered following the therapeutic agent.

The invention also features, in another aspect, a method of treating a subject having a tumor, comprising administering oxygen to the subject in an amount sufficient to reduce the size of the tumor, the volume of the tumor, and/or the number of tumor cells, wherein the oxygen is administered in a hyperbaric chamber or is administered as supplemental oxygen.

In some embodiments, the tumor to be treated is greater than about 2 mm in diameter. In certain embodiments, the size of the tumor to be treated is greater than about 0.5 mm in diameter, greater than about 1.0 mm in diameter, greater than about 1.5 mm in diameter, greater than about 2.0 mm in diameter, greater than about 2.5 mm in diameter, greater than about 3.0 mm in diameter, greater than about 4.0 mm in diameter, or greater than about 5.0 mm in diameter.

In some embodiments, the tumor to be treated has localized hypoxia areas. In certain embodiments, the tumor to be treated is a tumor of the kidney, urinary tract, colon, rectum, lung, liver, breast, prostate, or skin, or another tumor that is recognized by immune cells and that has tumor-infiltrating T cells.

In some embodiments, the oxygen increases the activity of tumor-infiltrating lymphocytes (“TILs”). In one embodiment, the activity is an enhanced anti-tumor activity. In certain embodiments, the anti-tumor activity is a cytotoxic activity of TILs or a secretion of cytokines. In particular embodiments, the secreted cytokines disrupt the blood supply to the tumor or prevent the formation of new blood vessels that supply blood to the tumor.

In some embodiments, the oxygen decreases immunosuppressive activities of T regulatory cells (Tregs).

In other embodiments, the method further comprises the step of evaluating the size of the tumor, the volume of the tumor, and/or the number of tumor cells after oxygen administration. In some embodiments, the size of the tumor, the volume of the tumor, and/or the number of tumor cells are evaluated before, during, and/or after oxygen administration. In certain embodiments, the oxygen is administered until the tumor is reduced to a preselected size, volume, or number of cells.

In one embodiment, the oxygen is administered in an amount and for a time to reduce the size of the tumor, the volume of the tumor, and/or the number of tumor cells, compared to the size, volume, and/or number of tumor cells prior to administration of oxygen. In certain embodiments, the oxygen administration reduces the size of the tumor, the volume of the tumor, and/or the number of tumor cells to less than 100%, to less than 95%, to less than 90%, to less than 80%, to less than 70%, to less than 60%, to less than 50%, to less than 30%, or to less than 10% of its size, volume, or cell number prior to therapy. In some embodiments, the oxygen administration reduces the growth of the tumor. In certain embodiments, the oxygen administration reduces the growth rate of the tumor by 10%, by 20%, by 30%, by 40%, by 50%, by 60%, by 70%, by 80%, by 90%, or by more than 90%, as compared to the growth rate of the tumor prior to oxygen administration.

In other embodiments, the method further comprises administering a therapeutically effective amount of a therapeutic agent to the subject. In certain embodiments, the therapeutic agent is an oxygen-enhancing substance that increases local oxygen tension in cancerous tissue in the subject. In some embodiments, the therapeutic agent is an A2a or A2b adenosine receptor antagonist. In some embodiments, the therapeutic agent is a Gi-coupled adenosine receptor agonist. In some embodiments, the therapeutic agent is an inhibitor of extracellular adenosine. In some embodiments, the therapeutic agent is an agent that decreases inflammation-associated local tissue hypoxia or decreases the redox status of molecules in an inflamed local tissue environment.

In certain embodiments, the therapeutic agent is an anti-tumor agent. In certain embodiments, the anti-tumor agent selectively targets the cells of the tumor. In particular embodiments, the anti-tumor agent is a nucleic acid molecule that encodes a protein that promotes apoptosis. In certain embodiments, the anti-tumor agent is an alkylating drug, a folate antagonist, a purine antagonist, a pyrimidine antagonist, a spindle poison, a podophyllotoxin, an antibiotic, a nitrosurea, an inorganic ion, a biologic response modifier, an enzyme, or a hormone.

In some embodiments, the oxygen is administered in combination with one or more therapeutic agents. In certain embodiments, the oxygen is administered in combination with (i) an A2a adenosine receptor antagonist or A2b adenosine receptor antagonist, and (ii) an A1 adenosine receptor agonist or an A3 adenosine receptor agonist.

In one embodiment, the oxygen and the therapeutic agent are administered concurrently. In another embodiment, the oxygen and the therapeutic agent are administered sequentially. In certain embodiments, the oxygen is administered prior to the therapeutic agent. In other embodiments, the oxygen is administered following the therapeutic agent. In another embodiment, the method further comprises administering oxygen in combination with surgery, cryosurgery, radiation therapy, thermotherapy, hormone therapy, chemotherapy, administration of a vaccine, or administration of an antibody.

In another aspect, the invention features a method of ablating or killing tumor cells, comprising administering oxygen to the tumor cells in an amount sufficient to ablate or kill the tumor cells, wherein the oxygen is administered in a hyperbaric chamber or as supplement oxygen.

In some embodiments, the method further comprises the step of evaluating the size or volume of the tumor, and/or the number of tumor cells after oxygen administration.

In one embodiment, the oxygen is administered in an amount and for a time sufficient to kill or ablate tumor cells. In certain embodiments, killing or ablating of the tumor cells is measured by a reduction in the size of the tumor, the volume of the tumor, and/or the number of tumor cells.

In other embodiments, the method further comprises administering a therapeutically effective amount of a therapeutic agent to the subject.

In another aspect, the invention features a method of disrupting the blood supply to a tumor in a subject, comprising administering oxygen to the subject in an amount sufficient to disrupt the blood supply to the tumor, wherein the oxygen is administered in a hyperbaric chamber or as supplemental oxygen.

In certain embodiments, the method of disrupting the blood supply to a tumor further comprises the step of evaluating the size or volume of the tumor, and/or the number of tumor cells after oxygen administration.

In one embodiment, the oxygen is administered in an amount and for a time sufficient to disrupt the blood supply to a tumor. In certain embodiments, disrupting the blood supply is measured by a reduction in the size of the tumor, the volume of the tumor, and/or the number of tumor cells in the subject.

In other embodiments, the method further comprises administering a therapeutically effective amount of a therapeutic agent to the subject.

In another aspect, the invention features a method of inducing or enhancing an immune response in a subject. The method comprises administering to the subject (i) a vaccine that elicits an immune response, and (ii) oxygen in a hyperbaric chamber or as supplemental oxygen, wherein the oxygen induces or enhances the immune response stimulated by the vaccine.

In some embodiments, the vaccine comprises an antigenic polypeptide or an antigenic epitope thereof. In certain embodiments, the vaccine is a viral vaccine. In particular embodiments, the viral vaccine is a live, attenuated, or heat killed viral vaccine. In some embodiments, the vaccine induces anti-tumor or anti-pathogen T cells.

In some embodiments, the subject is immunocompromised. In certain embodiments, the subject is infected with human immunodeficiency virus (HIV). In other embodiments, the subject is receiving immunosuppressive therapy. In other embodiments, the subject is infected with a virus, bacterium, or fungus.

In other embodiments, the method further comprises the step of evaluating the subject for a marker of an induced or enhanced immune response.

In another aspect, the invention features a method of producing a tumor defense-resistant immune cell or an anti-viral immune cell, comprising culturing an immune cell under hypoxic culture conditions to produce an immune cell that is resistant to hypoxia-produced extracellular adenosine, thereby producing a tumor defense-resistant immune cell or an anti-viral immune cell. In some embodiments, the immune cell is a cytotoxic T lymphocyte (CTL) or a lymphokine-activated killer (LAK) cell.

In certain embodiments, the hypoxic culture conditions comprise less than 4% oxygen. In particular embodiments, the hypoxic culture conditions comprise between 0.5% and 5% oxygen, between 1% and 4% oxygen, between 1% and 3% oxygen, or between 1% and 2% oxygen.

In another aspect, the invention features an isolated tumor defense-resistant immune cell or anti-viral immune cell produced by culturing an immune cell under hypoxic culture conditions. In some embodiments, the immune cell is a cytotoxic T lymphocyte (CTL) or a lymphokine-activated killer (LAK) cell.

In another aspect, the invention features a method of treating a subject having a tumor. In this method, one or more tumor defense-resistant immune cells are administered to the subject, thereby reducing tumor size, volume, and/or number of tumor cells. In some embodiments, the tumor defense-resistant immune cells are produced by culturing an immune cell under hypoxic culture conditions. In some embodiments, the immune cell is a cytotoxic T lymphocyte (CTL) or a lymphokine-activated killer (LAK) cell.

In some embodiment, the method of treating a patient further comprises monitoring the progress of the treatment, comprising: a) obtaining a biological sample from said subject, and b) determining the expression level of at least one marker indicative of an immune response to the tumor in the biological sample; wherein an altered expression level of the marker in the biological sample, as compared to a control, is indicative of an altered immune response to the tumor in the subject. The marker may be, for example, interferon gamma, interferon beta, interferon alpha, IL-12p40, TNF-alpha, or IL-17. The control may be an untreated subject, the subject prior to treatment, the subject at an earlier time point during treatment, or a database reference.

In another aspect, the invention features a method of enhancing an immune response to a virus in a subject. In this method, one or more anti-viral immune cells are administered to the subject, thereby enhancing the immune response to the virus in the subject. In some embodiments, the anti-viral immune cells are produced by culturing an immune cell under hypoxic culture conditions. In some embodiments, the immune cell is a cytotoxic T lymphocyte (CTL) or a lymphokine-activated killer (LAK) cell.

Another aspect of the invention provides a method for enhancing treatment of a cancer patient involving administering one or more of oxygen, an adenosine pathway antagonist or a HIF-1α antagonist, in conjunction with radiation therapy, ultrasound ablation, thermal ablation, electrical ablation, surgical excision, cryotherapy, laser therapy, phototherapy and the like.

Yet another aspect of the invention provides a combined therapy and vaccination methods to improve an enhanced immune response from an immunogen by step-wise and biomarkers-informed cumulative and escalated disengagement of individual sequential stages of immune response-inhibiting hypoxia-adenosinergic pathway.

In preferred embodiments, the adenosine receptor pathway antagonist is an adenosine receptor 2A (A2AR) antagonist. In an especially preferred embodiment, the A2AR antagonist is a small molecule that binds to A2AR. As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

The A2AR gene has multiple exons and is subject to alternative splicing. In addition, the gene has at least four alternative promoters. Thus, there are multiple A2AR isoforms. The compositions and methods herein may relate to all A2AR isoforms, or to a specific subset of them.

### BRIEF DESCRIPTION OF THE DRAWINGS

FIG. 1. FIG. 1A is a graphic representation of RMA tumor growth in wild type mice. FIG. 1B is a graphic representation of RMA tumor growth in A2AR-deficient mice.

FIG. 2 is a graphic representation of RMA tumor growth in the presence or absence of caffeine.

FIG. 3. FIG. 3A is graphic representation of survival rates of mice challenged with a high dose of RMA cells in the presence of normal oxygen, 60% oxygen, or 60% oxygen and caffeine. FIG. 3B is a graphic representation of survival rates of mice challenged with a low dose of RMA cells in the presence of normal oxygen (with or without caffeine) or 60% oxygen (with or without caffeine).

FIG. 4. FIG. 4A is a graphic representation of the production of TNP-specific IgM in immunized mice housed in either normal oxygen conditions or in 60% oxygen. FIG. 4B is a graphic representation of the production of TNP-specific IgM in immunized mice housed in 60% oxygen with or without caffeine administration.

FIG. 5 shows that treatment with the A2_{A}-specific antagonist KW6002 and high (60% vs normal 21% oxygen content) oxygen atmosphere can significantly retard tumor growth. Left panel, plot of tumor size vs time. Right panel, plot of mouse survival versus time.

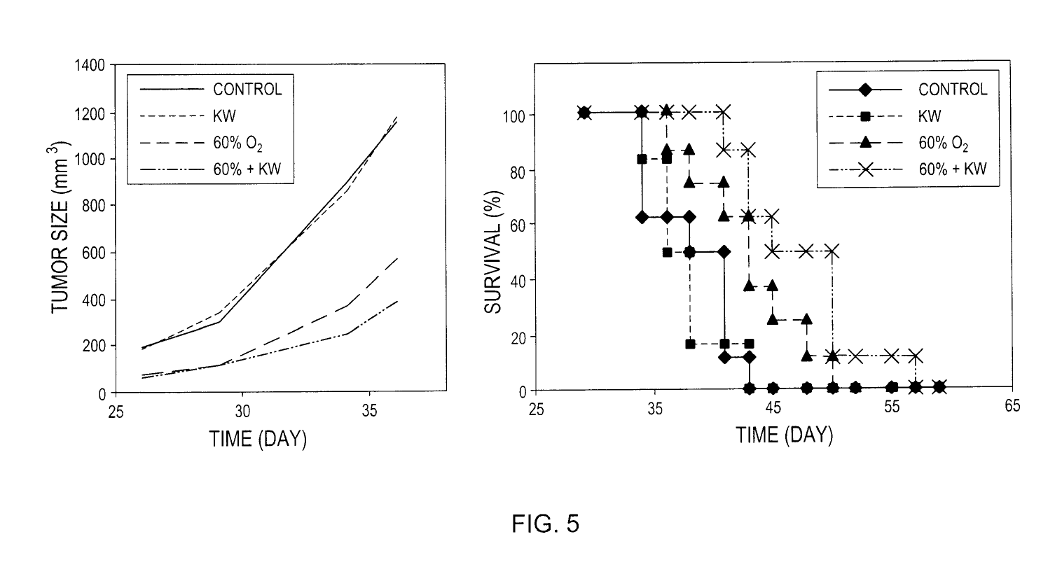

FIG. 6 shows that an A2_{A} antagonist can enhance the production of specific antibodies of different classes of immunoglobulins. Left panel, IgM levels. Right panel, IgG levels.

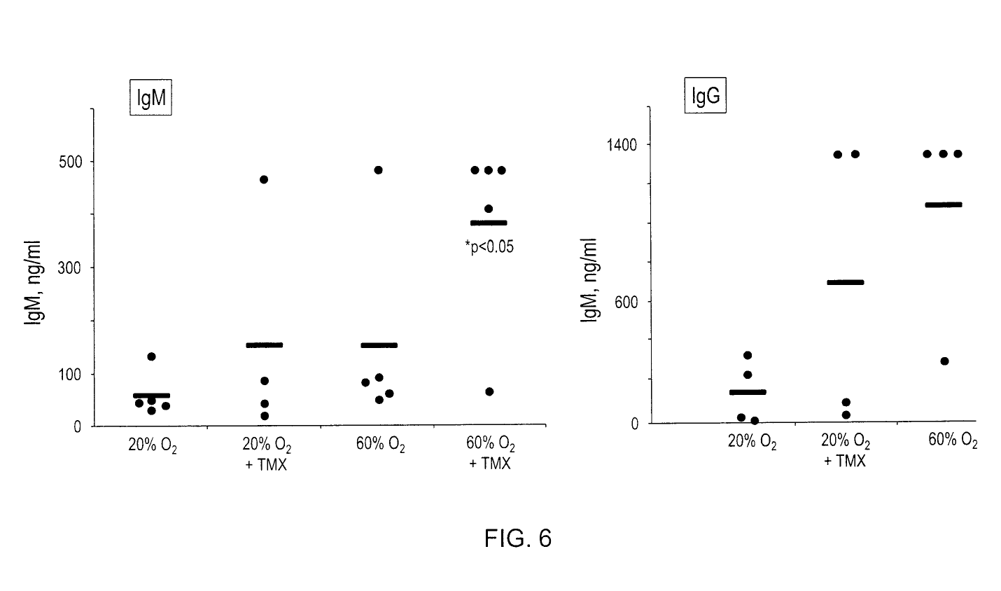

FIG. 7 depicts lung metastases from mice that were treated with KW 6002 with or without CTL.

FIGS. 8A and 8B depicts the survival rate of mice that received RNA T-lymphoma cells and were then treated with excess oxygen alone or excess oxygen combined with caffeine.

FIG. 9 depicts the tumor diameter in mice injected with MCA 205 fibrosarcoma. Mice were either deficient for A2AR, A2BR, or both.

FIG. 10 depicts the effects of NECA (an antagonist of A2AR and A2BR), CGS21680 (a specific inhibitor of A2AR), and ZM241385 (an A2AR and A2BR antagonist) on cAMP levels in human and murine iNKT cells.

FIG. 11 depicts several A2AR antagonists known in the art.

### DETAILED DESCRIPTION OF THE INVENTION

### I. Overview

In one aspect, the present invention relates to compositions and methods for enhancing an immune response to a vaccine by combining the administration of oxygen (O₂ gas), an adenosine pathway antagonist and/or an HIF-1α antagonist with the administration of the vaccine to the patient. For instance, the vaccine can be administered in conjunction with administering oxygen, an adenosine pathway antagonist and/or an HIF-1α antagonist to the patient.

The present invention also features methods of inducing or enhancing immune responses, methods of treating subjects having a tumor, methods of ablating or killing tumor cells, methods of disrupting the blood supply to a tumor, tumor defense-resistant immune cells and methods of their production, and anti-viral immune cells and methods of their production.

The present invention also relates to compositions and methods for enhancing the response of patients to radiation therapy, ultrasound ablation, thermal ablation, electrical ablation, surgical excision, cryotherapy, laser therapy, phototherapy and the like. For instance, such procedures can be carried out in conjunction with administering oxygen, an adenosine pathway antagonist and/or an HIF-1α antagonist to the patient.

The phrase “in conjunction with” when used in reference to the use oxygen, an adenosine pathway antagonist and/or an HIF-1α antagonist with a vaccine indicates that the agent and vaccine are administered so that there is at least some chronological overlap in their physiological activity on the patient. Thus the agents can be administered simultaneously and/or sequentially relative to administration of the vaccine. In sequential administration there may even be some substantial delay (e.g., minutes or even hours or days or weeks) between administration.

### II. Oxygen Administration

In the methods described herein, oxygen can be administered in a hyperbaric chamber or as supplemental oxygen. The administration of oxygen in a hyperbaric chamber is also referred to as hyperbaric oxygen therapy (“HBOT”). In HBOT, a subject is placed in a hyperbaric chamber and is administered 100% oxygen at a pressure that is greater than atmospheric pressure at sea level. Hyperbaric chambers have been available for many years and are known in the art (see, e.g., U.S. Pat. No. 4,727,870, U.S. Pat. No. 6,016,803, U.S. Pat. No. 6,321,746, U.S. Pat. No. 6,484,716). The methods described herein are not limited to the use of any particular hyperbaric chamber. Hyperbaric chambers can be commercially obtained from, for example, Parry Baromedical Corporation (Riviera Beach, Fla.) or Performance Hyperbarics (Kula, Hi.). Oxygen can also be administered in a hyperbaric chamber at a hyperbaric oxygen facility or clinic. One of ordinary skill in the art would readily appreciate the steps to take to deliver hyperbaric oxygen in accordance with the methods described herein (see, e.g., Tibbles et al., New England J. Med. 334:1642-1648, 1996).

In other methods described herein, oxygen is administered as supplemental oxygen. The use of supplemental oxygen is known in the art (see, e.g., Tarpy et al., N. Engl. J. Med. 333:710-714, 1995). Generally, supplemental oxygen therapy is administered from an oxygen concentrator or in the form of compressed gas or liquid oxygen. Subjects usually receive oxygen through a nasal cannula, but other devices such as nasal catheters, transtracheal catheters, and electronic demand devices can also be used. One of ordinary skill in the art would readily appreciate how to use and manipulate supplemental oxygen devices to deliver oxygen in accordance with the methods described herein, and these methods are not limited to the use of any particular supplemental oxygen device. For example, oxygen can be administered using a protocol similar to that described in Kabon et al., Curr. Opin. Anaesthesiol. 19:11-18, 2006.

In yet other methods described herein, oxygen is administered through a mask. Numerous masks have been described in the art. For example, plastic oxygen masks are frequently used in a health care setting. These masks do not deliver a high concentration of oxygen to the patient. Silicone and rubber masks provide tighter seals than plastic masks, and consequently can deliver a higher concentration of oxygen. Such masks have valves to prevent re-breathing of exhaled carbon dioxide. Such masks are used, for example, by aviators. Silicone and rubber masks can be classified into three main groups: continuous flow masks (which, as the name implies, provide an uninterrupted supply of oxygen), “diluter demand” masks (which provide oxygen only when the user inhales) and “pressure demand” masks (which provide oxygen only when the user inhales and are used when the ambient air pressure is low, for example at very high altitudes). An oxygen mask may be attached to a tank containing compressed oxygen, including liquid oxygen.

In certain embodiments, oxygen is delivered to a patient without mechanical ventilation. In certain embodiments, oxygen is delivered to a patient without intubation.

By “oxygen concentration” is meant FiO₂, or the fractional concentration of oxygen in inspired air, measured as volume per volume.

Oxygen can be administered daily or several times a day over a period of a few days to months, or even years. A therapeutically effective amount of oxygen can be the amount of oxygen necessary to stimulate the immune system of a subject. Specific immunostimulatory effects that can be caused by oxygen administration as well as specific immunosuppressive effects that can be caused by oxygen administration are described herein. In some embodiments, an immunostimulatory amount of oxygen is an amount sufficient to stimulate an immune response (such as an immune response described herein) without causing a substantial cytotoxic effect (such as without killing more than about 10% of cells in a sample). As used herein, the term “about” means a numeric value having a range off 10% around the cited value.

The subject to whom oxygen is administered can be monitored for one or more signs of oxygen toxicity. For example, a subject can be monitored for one or more of nausea, vomiting, seizures, sweating, pallor, muscle twitching, anxiety, respiratory changes, visual changes, tinnitus, hallucinations, vertigo, hiccups, decreased level of consciousness, dry cough, substernal chest pain, bronchitis, shortness of breath, pulmonary edema, or pulmonary fibrosis. The subject can be monitored at any time, e.g., before, during, and/or after oxygen administration.

### III. Compositions and Methods for Inducing or Enhancing Immune Responses

The invention includes, in part, compositions and methods for inducing or enhancing an immune response in a subject. The method comprises administering oxygen to the subject in an amount sufficient to induce or enhance the immune response. The oxygen is administered in a hyperbaric chamber or is administered as supplemental oxygen, as described above.

The immune responses that can be induced or enhanced by this method can be cell-mediated immune responses and/or humoral immune responses. The cell-mediated immune response can be mediated by one or more of a macrophage, granulocyte, monocyte, neutrophil, dendritic cell, T cell, B cell, or natural killer cell in the subject. For example, the cell-mediated immune response can be a cell-mediated cytolytic immune response. The immune responses induced or enhanced by the methods described herein can, in some cases, be mediated by one or both of CD4⁺ and CD8⁺ T cells.

The immune response can be induced or enhanced by increasing the secretion of a cytokine, e.g., a pro-inflammatory cytokine such as IL-2, IL-4, IL-12p40, and/or TNF-alpha. In some embodiments, the increase secretion of cytokines is due to increased NF-κB activity in the subject. Cytokines may also be administered therapeutically to the patient. In a preferred embodiment, the cytokines are inflammatory cytokines.

The subject can be evaluated for a marker of an induced or enhanced immune response, e.g., by determining the level of a pro-inflammatory cytokine described herein in blood or urine from the subject. One of skill in the art can readily identify methods to measure for an increased activity of an immune cell. For example, the level of one or more cytokines in the blood or urine from a subject can be measured by ELISA or PCR-based assays or in biological assays. In one example, the increase in activity is measured as compared to the activity of a control cell. Suitable controls include an immune cell from a subject that has not been administered oxygen, or an immune cell from a subject prior to the administration of oxygen, or a standard value. The subject can be evaluated before, during, and/or after administration of oxygen. Oxygen therapy can be administered until a predetermined level of an immune response is achieved.

The subject treated by this method can be a mammal such as a human or other vertebrate. The subject may be infected with a pathogen such as a virus, a bacterium, or a parasite. Exemplary viruses include, but are not limited to, HIV, West Nile virus, and Dengue virus. Exemplary bacteria include, but are not limited to, Mycobacteria, Rickettsia, and Chlamydia. Exemplary parasites include, but are not limited to, Plasmodium, Leishmania, and Taxoplasma. The subject may be an immunosuppressed, for example, a subject infected with an immunodeficiency virus (e.g., HIV-1 or HIV-2) or having or suffering from another immune deficiency (e.g., a deficiency of one or more types of immune cells, or of one or more immunological factors) associated with an immune deficiency disease such as SCID, an immune suppressive medical treatment, an acute and/or chronic infection, and aging. A general overview of immunosuppressive conditions and diseases can be found in Harrison's Principles of Internal Medicine, 14th Edition, McGraw-Hill, 1998, Chapters 86 (“Principles of Cancer Therapy”), 307 (“Primary Immune Deficiency Diseases”), and 308 (“Human Immunodeficiency Virus Diseases”).

As used herein, a subject “having” or who “has” a disease or disorder refers to a subject who has the clinical manifestations and/or symptoms of a disease or disorder. In certain situations, a subject with a disease or disorder may be asymptomatic, and yet still have clinical manifestations of the disease or disorder. For example, a subject suffering from leukemia may not be symptomatic (e.g., may not be sick or weak), but shows the clinical manifestation in that the subject has a larger number of white blood cells as compared to a healthy individual of the same age and weight. In another non-limiting example, a subject suffering from infection with a virus (e.g., HIV) may not be symptomatic (e.g., may not show a diminished CD4⁺ T cell count), but shows the clinical manifestation in that the subject has anti-HIV antibodies.

Sometimes, oxygen may be administered to subjects who have undergone or are undergoing a medical treatment that can impair the immune system. Corticosteroids, for example, as a medical treatment can reduce cell-mediated immunity. The predominant toxicity associated with cancer therapies (e.g., chemotherapy and radiotherapy) can involve the destruction of proliferating cells, such as hematopoietic cells, responsible for maintenance of the immune and blood systems. Likewise, immune suppression and depletion of the immune system is required for bone marrow transplantation, in which immune cells are eliminated and subsequently replaced with transplanted cells. Certain known immunostimulants (e.g., erythropoietin and colony stimulating factors such as G-CSF, which is sometimes marketed under the name “Neupogen,” U.S. Pat. No. 5,536,495) have been used previously to treat certain of these conditions by stimulating regeneration of the immune cells.

The need for oxygen administration can be determined by examining the immune status of a test subject, and comparing this immune status to a control or average immune state (a hypothetical “normal” subject). For example, bone marrow biopsies or peripheral blood lymphocytes can be sampled to assess immune function. Indications of reduced immune function include leucopenia, for example, neutrophenia or lymphopenia, or evidence of diminished white blood cell function. In some situations, oxygen is administered to a subject who has a reduced immunity condition, such as a reduction in a peripheral white blood cell count to below normal, for example, 25% below normal.

### IV. Compositions and Methods for Treating Tumors

A. Methods of Treating Tumors

The invention includes, in part, methods of treating a subject having a tumor. The method comprises administering oxygen to the subject, wherein the oxygen is administered in a hyperbaric chamber or is administered as supplemental oxygen. The use of hyperbaric chambers and supplemental oxygen is described herein. The administration of oxygen can increase inflammatory actions of immune cells (such as tumor-infiltrating lymphocytes). The administration of oxygen can additionally promote the recruitment of other immune cells with anti-tumor activity to improve the destruction of a tumor (such as by reducing the size of the tumor, the volume of the tumor, and/or the number of tumor cells). The administration of oxygen can improve both natural anti-tumor immune responses and adaptive immunotherapy of tumors by immune cells that recognize tumor-associated antigens on the tumor cell surface. These anti-tumor or anti-pathogen responses may include, for example, increased differentiation, increased expansion, and/or improved effector functions of endogenously developed or adoptively transferred anti-tumor or anti-pathogen T cells or myeloid cells. These immune cells are capable of recognizing tumors or participating in enhanced production of cytokines and/or chemokines with anti-tumor or anti-pathogen activities.

The term “administering” includes routes of administration which allow the vaccine or other composition of the invention to perform its intended function, e.g. stimulate an immune response. Preferred routes of administration include, but are not limited to, intramuscular, intraperitoneal, oral, intrabronchial, and transdermal. Depending on the route of administration, the vaccine of the invention can be coated with or disposed in a selected material to protect it from natural conditions which may detrimentally effect its ability to perform its intended function. The vaccine of the invention can be administered alone or with a pharmaceutically acceptable carrier. Further, the vaccine and adenosine pathway antagonist and/or an HIF-1α antagonist can be administered as a mixture, which also can be coadministered with a pharmaceutically acceptable carrier.

As used herein, “treat”, “treating” or “treatment” refers to administering a therapy in an amount, manner (e.g., schedule of administration), and/or mode (e.g., route of administration), effective to improve a disorder or a symptom thereof, or to prevent or slow the progression of a disorder or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

As used herein, “tumor” or “neoplasm” means an abnormal mass of tissue that results from excessive cell division that is uncontrolled and progressive. Tumors can be benign (neither infiltrative nor cancerous) or malignant (invasive).

As used herein, a “tumor cell” or “neoplastic cell” is a cell that shows aberrant cell growth, such as increased cell growth. Non-limiting examples of tumor cells include lymphoma cells, melanoma cells, breast cancer cells, ovarian cancer cells, prostate cancer cells, sarcoma cells, leukemic cells, retinoblastoma cells, hepatoma cells, myeloma cells, glioma cells, mesothelioma cells, and carcinoma cells.

Cancers that can be treated according to the methods of the invention include, but are not limited to, leukemia (e.g., acute leukemia such as acute lymphocytic leukemia and acute myelocytic leukemia, Chronic Lymphocytic Leukemia (CLL),), Chronic Myelogenous Leukemia (CML), and Hairy Cell Leukemia (HCL)), neoplasms, tumors (e.g., Hodgkin's lymphoma, non-Hodgkin's lymphoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma), heavy chain disease (B-cell lymphoma), T cell cancers, natural killer cell cancers, metastases, or any disease or disorder characterized by uncontrolled cell growth.

B. Exemplary Tumor Vaccines and Target Diseases

A wide variety of vaccines may be used in connection with methods of this invention. For example, any cancer vaccine or pathogen vaccine may be used. Target diseases for methods of the invention includes cancer, as well as infectious or inflammatory diseases.

To further illustrate, the methods and compositions of the invention can be used in the treatment of cancers, including, but not limited to, neoplasms, tumors, metastases, or any disease or disorder characterized by uncontrolled cell growth. Specific examples of cancer include, but are not limited to: cancers of the skin, such as melanoma; lymph node; breast; cervix; uterus; endometrium; gastrointestinal tract; lung; ovary; prostate; colon; rectum; mouth; brain; head and neck; throat; testes; kidney; pancreas; bone; spleen; liver; bladder; larynx; nasal passages; and AIDS-related cancers. Methods of the invention are particularly useful for treating cancers of the blood and bone marrow, such as multiple myeloma and acute and chronic leukemias, for example, lymphoblastic, myelogenous, lymphocytic, myelocytic leukemias, and myelodysplastic syndromes including but not limited to 5 q minus syndrome, or myelodysplastic syndromes associated with other cytogenic abnormalities. The methods of the invention can be used for treating, preventing or managing either primary or metastatic tumors.

Other specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma (localized melanoma, including, but not limited to, ocular melanoma), malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage 1V non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In a specific embodiment, the cancer is metastatic. In another embodiment, the cancer is refractory or resistance to chemotherapy or radiation.

Tumor antigens or tumor associated antigens include cancer-germ cell (CG) antigens (MAGE, NY-ESO-1), mutational antigens (MUM-1, p53, CDK4), over-expressed self-antigens (p53, HER2/NEU), viral antigens (from Papilloma Virus, Epstein-Barr Virus), tumor proteins derived from non-primary open reading frame mRNA sequences (NY-ESO1, LAGE1), Melan A, MART-1, MAGE-1, MAGE-3, BAGE, GAGE-1, GAGE-2, tyrosinase, gp100, gp75, HER-2/neu, c-erb-B2, CEA, PSA, MUC-1, CA-125, Stn, TAG-72, KSA (17-1A), PSMA, p53 (point mutated and/or overexpressed), RAS (point mutated), EGF-R, VEGF, GD2, GM2, GD3, Anti-Id, CD20, CD19, CD22, CD36, Aberrant class II, B1, CD25 (IL-2R) (anti-TAC), or HPV.

TABLE 1
Exemplary vaccines for use in the subject methods and compositions.

| | Name of | | |
|---|---|---|---|
| Type of cancer | vaccine | Platform | Company |
| B-cell Lymphoma | Favld | KLH-idiotype and | Favrille |
| | | GM-CSF | |
| Breast cancer | PX 104.1 | HER2 protein | Pharmexa A/S |
| Cervical cancer | Lovaxin C | HPV-E7 expressed by | Advaxis |
| and head and neck | | Listeria vector | |
| cancer | | | |
| Cervical dysplasia | ZYC-101A | DNA-based | MGI Pharma |
| | | | Biologics |
| Cervical dysplasia | TG-4001 | MVA virus encoding | Transgene SA |
| and uterine cervix tumor | | HPV type 16 E6 and | |
| | | E7 antigens and IL-2 | |
| Colorectal cancer | OncoVax-CL | Iraddiated autologus | Intracel |
| | | tumor | |
| Head and neck | INGN-201 | Adenovirus | Introgen |
| cancer | | expressing | Therapeutics |
| | | p53 | |
| Melanoma | Vitespen | Autologous Hsp90B1 | Antigenics |
| | | gp96 | |
| Melanoma | GMK | GM2 ganglioside and | Progenics |
| | | KLH-idiotype | Pharmaceuticals |
| Melanoma | IDD-3 | Autologous | IDM Pharma |
| | | dendritphages loaded | |
| | | with TAA | |
| Melanoma | CYT-004- | MelanA/MART1 | Cytos |
| | MelQbG10 | protein fragments | Biotechnology |
| Melanoma | Hi-8MEL | MVA virus encoding | Oxxon |
| | | melanoma TAA | Therapeutics |
| Non-Hodgkin's | MyVax | KLH-idiotype | Genitope |
| lymphoma | | and GM-CSF | |
| Non-small cell | BLP-25 | MUC-1 peptide based | Merck |
| lung cancer | | liposome | |
| Non-small-cell | TG-4010 | MVA virus encoding | Transgene SA |
| lung cancer | | MUC-1 and IL-2 | |
| Ovarian cancer | CVac | Mannan adjuvant | Prima BioMed |
| | | attached to MUC1 | |
| Pancreatic cancer, | GV-1001 | Telomerase peptide | GemVax AS |
| Solid tumor | | | |
| Prostate cancer | Sipuleucel-T | PAP-pulsed patient | Dendreon |
| | | DC | |
| Prostate cancer | DCVax-Prostate | rPSMA-pulsed patient | Northwest |
| | | DC | Biotherapeutics |
| Prostate cancer | Pentrys | p53-based peptide | Avantogen |
| Prostate cancer | Onyvax-P | Inactivated prostate | Onyvax |
| | | tumor cell lines | |
| Prostate cancer | GRNVAC1 | Telomerase DC | Geron |
| Prostate cancer | Uvidem | Dendritophages | IDM Pharma |
| | | | inc/sanofi- |
| | | | aventis |
| Prostate cancer, | GVAX | Tumor cell line and | Cell Genesys |
| leukemia and | | GM-CSF | |
| pancreatic cancer | | | |
| Range of cancer | ZYC-300 | DNC encoded | MGI Pharma |
| types | | CYP1B1 | Biologics |
| Renal cell cancer | TroVax | MVA virus encoding | Oxford |
| | | tumor antigen 5T4 | Biomedica |
| Renal tumor and | AGS-003 | Tumor RNA and DC | Argo |
| chronic | | | Therapeutics |
| lymphocytic | | | |
| leukemia | | | |
| Small-cell lung | INGN-225 | p53 tumor antigen | Introgen |
| cancer and breast | | DCs | Therapeutics |
| tumors | | | |
| Solid Tumor | INGN-241 | Adenovirally | Introgen |
| | | delivered MDA-7 | Therepeutics |
| Prostate cancer | Provenge | Active Cellular | Dendreon |
| | | Immunotherapy | Corporation |
| Lung, breast, and | Neuvenge | Active Cellular | Dendreon |
| other cancers | | Immunotherapy | Corporation |
| Melanoma | M-Vax | Autologous cancer | Avax |
| | | cells | Technologies |
| Melanoma | Melacine | Allogenic tumor | Corixa |
| | | antigens | |
| Melanoma | Canvaxin | Allogenic tumor cells | CancerVax |
| Bladder cancer | PACIS | Bacillus Calmette- | Shire |
| | | Guérin (BCG) | Pharmaceuticals |
| Bladder cancer | TheraCys, | Bacillus Calmette- | Aventis |
| | ImmunoCys | Guérin (BCG) | |
| Bladder cancer | TICE BCG | Bacillus Calmette- | Akzo Nobel |
| | | Guérin (BCG) | |
| Colorectal cancer | CeaVac | Anti-idiotype mAb | Titan |
| | | | Pharmaceuticals |
| Colorectal cancer | Avicine | Peptide antigen | AVI |
| | | | BioPharma |
| Melanoma | Allovectin-7 | Gene for antigen | Vical |
| Melanoma | GMK | Ganglioside antigen | Progenics |
| | | | Pharmaceuticals |
| Non-Hodgkin's | (Idiotype | Recombinant protein | Genitope |
| lymphoma | immunotherapy) | antigen, patient | |
| | | specific | |
| Non-Hodgkin's | (Idiotype protein | Autologous protein | BioVest |
| lymphoma | vaccine) | antigen | International |
| Pancreatic, | Gastrimmune | Recombinant protein | Aphton |
| stomach, colorectal | | antigen | |
| cancers | | | |
| Small-cell lung | BEC2 | Anti-idiotype mAb | ImClone |
| cancer | | | |
| Renal cell cancer, | Oncophage | Autologous protein | Antigenics |
| melanoma | | antigen | |
| Non-small cell | PT 107 | Therapeutic vaccine | Pique |
| lung cancer | | | Therapeutics |

C. Compositions and Methods for Enhancing the Efficacy of Vasculature-Targeting Agents

The term “vasculature-targeting agent” is used herein to refer to an agent that alter the vasculature of a tumor. Said alteration may be disruption, inhibition, or normalization. Such agents include agents that inhibit the function of pre-existing vasculature around a tumor (for example, by collapsing it), agents that inhibit neovascularization, and agents that normalize pre-existing abnormal vasculature.

Solid tumors require oxygen and nutrients from blood, and tumors greater than a few cells in diameter require angiogenesis to survive. The tumor-associated vasculature is often abnormal both structurally and functionally, with excess endothelial cells forming twisting vasculature. The vasculature may also by hyper-permeable and dilated. Finally, the combination of impaired oxygen delivery and high oxygen consumption of the tumor creates a hypoxic environment within a tumor. The paucity of blood flow to the tumor also impedes therapeutic compounds in the bloodstream from reaching the tumor.

Tumor cells promote angiogenesis by the secretion of angiogenic factors, in particular basic fibroblast growth factor (bFGF) (Kandel J. et al., Cell, 1991, 66, 1095-1104) vascular endothelial growth factor (VEGF) (Ferrara et al., Endocr. Rev., 1997, 18: 4-25) and platelet derived growth factor (PDGF).

A number of drugs affect tumor vasculature. While the mechanism of such drugs is not fully understood, there appear to be three broad classes of vasculature-targeting agents. First, an agent may be anti-angiogenic. Such agents prevent the growth of new blood vessels, starving the tumor of blood and oxygen. Such agents make a tumor more hypoxic. Second, an agent may collapse pre-existing tumor vasculature, also increasing the hypoxia of the tumor. Third, vasculature-normalizing agents reduce the abnormalities of the tumor vasculature. For example, they may reduce the number of excess epithelial cells in the tumor vasculature. These agents improve blood flow to the tumor and reduce hypoxia. Paradoxically, vasculature-normalizing agents may be used to impede tumor growth, by allowing other therapeutic molecules (such as chemotherapeutic drugs) better access to the tumor.

Some therapies previously thought to be anti-angiogenic may instead produce vasculature normalization. For example, one may block vascular endothelial growth factor (VEGF) or its receptor (VEGFR2), causing apoptosis of endothelial cells. Consequently there is a decrease in blood vessel diameter, density and permeability. There is also a decrease in interstitial fluid pressure and, at least in some instances, elevated oxygen tension (reviewed in Jain R et al., Nature Medicine 7, 987-989 (2001)). Various other therapeutics also contribute to vasculature normalization, including STI571, C225, and Herceptin, which block PDGFR, HER1 and HER2 signaling, respectively.

Therapeutic antibodies may be used to normalize tumor vasculature. For example, a neutralizing antibody (A4.6.1) against VEGF/VPF is described in Yuan F et al. (Proc Natl Acad Sci USA. 1996 Dec. 10; 93(25):14765-70.) Permeabolization of the tumor vasculature was observed a few hours after injection and lasted about 5 days. Also, the (VEGFR)-2 neutralizing antibody DC101 may be used to normalize tumor vasculature as described in Kadambi et al., (Cancer Res. 2001 Mar. 15; 61(6):2404-8). Humanized versions of these antibodies, and antibody variants such as single-chain antibodies, may be used in accordance with the methods disclosed herein.

Angiostatin is a member of a family of anti-angiogenic plasminogen fragments (“AAPFs”). Physiologically relevant AAPFs include a 38 kDa AAPF isolated from the conditioned media of tumor-infiltrating macrophages (Dong et al. (1997) Cell 88, 801-810), a 43 kDa and 38 kDa AAPF identified in the conditioned media of Chinese hamster ovary and HT1080 fibrosarcoma cells and a 48 kDa AAPF present in macrophage conditioned media (Falcone et al. (1998) J. Biol. Chem. 273, 31480-31485). Other AAPFs include a 43 kDa and a 38 kDa AAPF isolated from the conditioned media of human prostrate carcinoma PC-3 cells (Gately et al. (1996) Cancer Res. 56, 4887-4890; Gately et al. (1997) PNAS USA 94, 10868-10872) and AAPFs of 66, 60 and 57 kDa detected in the conditioned media of HT1080 and Chinese hamster ovary cells (Stathakis et al. (1999) J Biol Chem 274, 8910-8916).

In certain embodiments, the vasculature-targeting agent is selected from one or more of the following; alpha interferon, angiogenic steroids, Bevacizumab Batimastat (BB-94), carboxyaminoimidazole (CAI), CM101 (GBS toxin), CT-2548, hydrocortisone/beta-cyclodextran, interleukin-12, Linomide, Marimastat (BB-2516), Octreotide (somatostatin analogue), Pentosan polysulfate, platelet factor 4, Roquinimex (LS-2616, linomide), Suramin, SU101, Tecogalan sodium (DS-4152), thalidomide and its derivatives, TNP-470 (AGM-1470), angiostatin, endostatin, tumstatin, Avastin, beta interferon, gamma interferon, cartilage-derived inhibitor (CDI), gamma interferon inducibile protein (IP-10), gro-beta, heparinases, placental ribonuclease inhibitor, plasmingoen activator inhibitor, proliferen-related protein, retinoids, thrombospondin, TIMP-2, and 16 kd prolactin. In certain embodiment, the vasculature-targeting agent is a bFGF or VEGF inhibitor. In other embodiments, the vasculature-targeting agent is a taxane such as taxol, docetaxel, or paclitaxel. While not wishing to be bound by theory, it is possible that low doses of taxol cause tumor vasculature to collapse.

In certain embodiments, immunostimulatory agents are administered to a patient simultaneously with a vasculature-targeting agent. In other embodiments, immunostimulatory agents are administered to a patient after a vasculature-targeting agent. This period of time may be 1, 2, 4, 6, 8, 16, or 24 hours, or 2, 3, 4, 5, 10, or days.

In certain instances it will be desirable to visualize the vasculature of the tumor in order to determine if or when to administer an immunostimulatory agent (such as an A2AR agonist) to the patient. Tumor vasculature can be visualized by any means known in the art. Exemplary methods include DUS (Doppler ultrasound) (Menon et al., “An Integrated Approach to Measuring Tumor Oxygen Status Using Human Melanoma Xenografts as a Model” Cancer Research 63, 7232-7240, Nov. 1, 2003)); Diffuse correlation spectroscopy (DCS) (Sunar et al., “Noninvasive diffuse optical measurement of blood flow and blood oxygenation for monitoring radiation therapy in patients with head and neck tumors: a pilot study.”, J Biomed Opt. 2006 November-December; 11(6):064021); xenon (Xe) inhalation detected by CT scans in human patients (Shimizu J et al, Noninvasive Quantitative Measurement of Tissue Blood Flow in Hepatocellular Carcinoma Using Xenon-Enhanced Computed Tomography, Dig Dis Sci. 2003 August; 48(8):1510-6); radiotracer methods involving labeled water (Bacharach et al., Measuring tumor blood flow with H₂¹⁵O: practical considerations, Nuclear Medicine and Biology Volume 27, Issue 7, October 2000, Pages 671-676); and multivoxel proton MR spectroscopic imaging (Chawla S et al., Arterial spin-labeling and MR spectroscopy in the differentiation of gliomas, AJNR Am J Neuroradiol. 2007 October; 28(9):1683-9. Epub 2007 Sep. 24.).

In certain instances, the overall status of the tumor is assayed using means that are known in the art, in order to determine if or when to administer an immunostimulatory agent (such as an A2AR agonist) to the patient. The overall status of a tumor may be assayed using, for example, cancer biomarkers, CT scans, or patient-reported symptoms like pain.

When an adenosine receptor antagonist is administered together with a vasculature-targeting agent, the timing of the doses may be selected as set out below. The adenosine receptor antagonist may be administered simultaneously with the vasculature-targeting agent. Alternatively, the adenosine receptor antagonist may be administered after the vasculature-targeting agent. For instance, the adenosine receptor antagonist may be administered 1, 2, 3, 4, 5, or more days after the vasculature-targeting agent. Furthermore, the time of adenosine receptor antagonist administration may be selected depending on the blood flow to the tumor. If an agent that restricts blood flow to the tumor has been administered, the adenosine receptor antagonist may be administered after the blood flow to the tumor is reduced 20%, 40%, 60%, or 80% or more. If an agent that increases blood flow to the tumor has been administered, the adenosine receptor antagonist may be administered after the blood flow to the tumor is increased 50%, 2-fold, 3-fold, or 5-fold or more. Furthermore, the vasculature targeting agent may be administered continuously or periodically, for example daily. In addition, the adenosine receptor antagonist may be administered continuously or periodically, for example daily.

Herein, the term “simultaneously” is used to encompass two events that occur at essentially the same time. For example, simultaneous administration of oxygen and an A2AR antagonist includes a situation where oxygen is administered continuously for several hours, and the A2AR antagonist is administered once during that period. In addition, simultaneous administration of oxygen and an A2AR antagonist includes a situation where oxygen and the A2AR antagonist are administered on the same day.

In certain embodiments, one may combine an immunostimulatory agent (as disclosed herein) in combination with an agent that breaks self-tolerance. Such an agent may be an IgG molecule or any agent known in the art to improve the immune system's recognition of a tumor that is largely recognized as “self” by the patient's immune system. Bone-marrow transplants may also be used to break self-tolerance. Other tolerance-breaking agents include IL2, and anti-CD28 antibodies; in certain embodiments, the anti-CD28 antibodies are altered to reduce toxicity.

D. Use of Biomarkers to Gauge Therapy Efficacy

The progression of a cancer, and the success of an anti-cancer therapy, may be gauged using biomarkers. A multitude of biomarkers are known in the art.

Any biomarker may be used with the methods described herein. By “biomarker” is meant a molecule that is present at different concentrations in a cancer cell, cancerous tissue, or patient with cancer, compared to a non-cancerous cell, non-cancerous tissue, or patient without cancer. For example, a biomarker may be a protein that is expressed more highly in a tumor than in the corresponding non-cancerous cell type. A biomarker may be a polypeptide, oligopeptide, lipid, carbohydrate, nucleic acid, small molecule, or a variant of any of these molecules (such as a phosphorylated protein or methylated DNA). Biomarkers may be identified using methods known in the art, including mass spectrometry and microarray technology.

Biomarkers that may be used to detect ovarian cancer include α-1-antitrypsin, AMBP, calgranulin B, carbonic anydrase, clusterin, cofilin (non-muscle isoform), ficolin 2, ficolin 3, gelsolin, haptoglobin, haptoglobin-related biomarker, hemopexin, inter-.alpha.-trypsin inhibitor, peptidyl-prolyl cis-trans isomerase A, plasma glutathione peroxidase, platelet basic protein, serotransferrin, serum amyloid A4 protein, tetranectin, transthyretin, vitronectin, and zinc-α-2-glycoprotein.

Biomarkers that may be used to detect liver cancer are disclosed in U.S. Patent Application No. 20050152908 and include amyloid beta (A4) precursor-like protein 2 (APLP2); BCL2-related protein A1 (BCL2A1); phosphoprotein regulated by mitogenic pathways (C8FW); CD14 antigen (CD14); Complement Component 5 (C5); C-type lectin-like receptor-2 (CLEC2); CDC-like kinase 1 (CLK1); Clusterin (CLU); cathepsin B (CTSB); cortactin (CTTN); ficolin (collagen/fibrinogen domain containing) 1 (FCN1); Putative lymphocyte G0/G1 switch gene (GOS2); interleukin 23A (IL23A); IGF-II mRNA-binding protein 3 (IMP-3); killer cell lectin-like receptor subfamily B, member 1 (KLRB1); 2′,5′-oligoadenylate synthetase 1 (OAS1); 2′-5′-oligoadenylate synthetase 3 (OAS3) RAR-related orphan receptor A (RORA); Related RAS viral (r-ras) oncogene homolog 2 (RRAS2) synuclein, alpha (non A4 component of amyloid precursor (SNCalif.); Homo sapiens serinethreonine kinase 17b (apoptosis-inducing) (STK17B); transcription factor EC (TFEC); and killer cell lectin-like receptor subfamily B.

Recently identified prostate cancer markers include PCTA-1 (Su et al., 1996, Proc. Natl. Acad. Sci. USA 93: 7252), prostate-specific membrane (PSM) antigen (Pinto et al., Clin Cancer Res 1996 Sep. 2 (9): 1445-51), STEAP (Hubert, et al., Proc Natl Acad Sci USA. 1999 Dec. 7; 96(25): 14523-8) and prostate stem cell antigen (PSCA) (Reiter et al., 1998, Proc. Natl. Acad. Sci. USA 95: 1735).

Some additional useful cancer biomarkers that have been identified are oncofetal antigens such as carcinoembryonic antigen (CEA) and alpha-fetoprotein, tissue-specific antigens such as prostate-specific antigen (PSA), and mucin antigens such as those conventionally known as CA-125 and CA-19-9. Immunoassays for antigens such as these are typically used as confirmatory tests at the time of diagnosis and subsequently for monitoring patient status. Occasionally, the use of such tests crosses the boundaries of tumor type (for example, the use of CEA tests in colon, breast, and lung cancer, and alpha-fetoprotein in hepatocellular and testicular cancer), but the utility of each test type is foremost for a single tumor type (for example, PSA for prostate cancer and CA-125 for ovarian cancer).

A family of antigenic proteins have been identified which are genetically and immunologically related to CEA (Thompson, J. and W. Zimmerman (1988) Tumor Biol. 9, 63-83; and Barnett, T. and W. Zimmerman (1990) Tumor Biol. 11, 59-63). Among these are the nonspecific cross-reacting antigens (NCAs), the trans-membrane antigens designated biliary glycoprotein (BGP, and sometimes referred to as TM-CEAs), and the family of pregnancy-specific .beta.-glycoproteins (PSGs) (for a description of the accepted nomenclature of these genes and their protein products, reference can be made to: Barnett, T. and W. Zimmerman (1990) Tumor Biol. 11, 59-63). Molecular cloning of the CEA gene family has enabled the identification of 22 members, of which 20 are probably expressed (Frangsmyr, L. et al. (1992) Tumor Biol. 13, 98-99; and Hammerstrom, S. et al Tumor Biol. 13, 57). The results of molecular genetic analysis have given a better understanding of the complex group of glycoproteins in the CEA gene family.

Biomarkers that may be used in accordance with the methods described herein include AMACR, PAP, PSM, and PSA (detecting prostate cancer), HER2 (breast cancer), CA-125 (ovarian cancer), Carcinoembryonic antigen (CEA) (colorectal, breast, lung, or pancreatic cancer), CA19-9 (pancreatic cancer, US Patent Application No. 20050095611), promoter region of GSTP1 (US Patent Application No. 20080026395), epigenetic markers, NGAL (atypical ductal hyperplasia, indicative of pre-breast cancer; US Patent Application No. 20070196876), CD97 or CD 55 (prostate cancer, US Patent Application No. 20070104717), COX4-2 (lung cancer, US Patent Application No. 20060257898), LAMA2 and other cited in US Patent Application No. 20060234254, Kallikrein 12, kallikrein 14, and kallikrein 15 (endocrine cancer, US Patent Application No. 20060223059), EPCA (prostate cancer, US Patent Application No. 20060148011), G-CSF mutations (US Patent Application No. 20050266430), leptin, prolactin, OPN and IGF-II (ovarian cancer, US Patent Application No. 20050214826), delta-catenin (US Patent Application No. 20050032099), ERRγ (breast cancer, US Patent Application No. 20040142490), hK10 (ovarian cancer, US Patent Application No. 20040115745), hK6 (ovarian cancer, US Patent Application No. 20040096915), GSTP1 (prostate cancer, US Patent Application No. 20030124600), alpha-haptoglobin (ovarian, US Patent Application No. 20030017515), PKC (colon cancer, US Patent Application No. 20010044113), calreticulin (urothelial cancer, U.S. Pat. No. 7,323,312), 125P5C8 (multiple cancers, U.S. Pat. No. 7,271,240), Nicotinamide N-methyltransferase (colorectal cancer, 7,205,118), ULIP proteins (U.S. Pat. No. 7,183,400), ITGβ6 (cervical cancer, U.S. Pat. No. 7,125,663), TIMP-1 (U.S. Pat. No. 7,108,983), Nup88 (U.S. Pat. No. 7,029,866), Csk autoantibodies (U.S. Pat. No. 6,759,204), VEGFR and Neuropilins (U.S. Pat. No. 6,635,421), COTA (colon cancer, U.S. Pat. No. 6,531,319), hnRNP protein (lung cancer, U.S. Pat. No. 6,500,625), hK2 (prostate cancer, U.S. Pat. No. 6,479,263), TSC403 (U.S. Pat. No. 6,403,785), NCA 50/90 (colon cancer and lung cancer; U.S. Pat. Nos. 6,309,846, 5,605,804)

Numerous other cancer biomarkers are known in the art (for a summary, see The Promises and Challenges of Improving Detection and Treatment, Sharyl J. Nass and Harold L. Moses, Editors, INSTITUTE OF MEDICINE OF THE NATIONAL ACADEMIES, THE NATIONAL ACADEMIES PRESS, and Hoffman B R, Diamandis E P., “Recent advances in cancer biomarkers.” Clin Biochem. 2004 July; 37(7):503-4)

One may measure the level of cancer progression in order to determine when to administer immunostimulatory therapy to a patient. For example, one might immunize a patient with a cancer vaccine, measure the levels of a cancer biomarker, and then administer an adenosine receptor antagonist when the marker indicates that the cancer biomarker levels have changed. In a preferred embodiment, an A2AR antagonist is administered when the cancer biomarker indicates that the cancer has progressed and therefore additional therapy is needed.

Appropriate biomarkers may also be used to assay immune system response. Such biomarkers include immunoglobulin levels (for example, IgA, IgG, IgM, IgE, IgD and various isoforms thereof), white blood cell counts, and measurements of various cytokines (such as IL-2 and TNFα).

Immunoglobulin levels can usually be detected in immunized individuals within a short time of vaccination. For example, after Rubella vaccination, specific IgG can be detected approximately 3 weeks after vaccination (Takahashi S et al, “Detection of Immunoglobulin G and A Antibodies to Rubella Virus in Urine and Antibody Responses to Vaccine-Induced Infection”, Clin Diagn Lab Immunol. 1998 January; 5(1): 24-27.) In addition, IgM levels peaked 3 weeks after vaccination of subjects with a measles vaccine (HELFAND R F et al, “The effect of timing of sample collection on the detection of measles-specific IgM in serum and oral fluid samples after primary measles vaccination” Epidemiology and Infection (1999), 123: 451-455 Cambridge University Press).

One may measure the level of an immune response in order to determine when to administer immunostimulatory therapy to a patient. For example, one might immunize a patient with a cancer vaccine, measure the levels of specific IgG that are raised to the immunogen, and then administer an adenosine receptor antagonist when IgG levels are high.

Cytokines include those in the IL-2, IFN, and IL-10 subfamilies. Cytokines include IL-4, IL-7, IL-9, IL-15, IL-21, IL-1, IL-17, IL-18, IFN-α, IFN-β, IFN-ω, IFN-γ, IL10R2, IFNLR1, TNFα, TGF-β1, TGF-β2, TGF-β3, and G-CSF, and GM-CSF.

### V. Compositions and Methods for Inducing or Enhancing Immune Responses to Vaccines

A. Inducing or Enhancing Immune Responses to Vaccines

The invention is also directed to a method of inducing or enhancing an immune response in a subject, comprising administering oxygen and a vaccine to the subject, wherein the oxygen induces or enhances the immune response stimulated by the vaccine, and wherein the oxygen is administered in a hyperbaric chamber or is administered as supplemental oxygen. The vaccine may be administered to the subject prior to, during, and/or after the administration of oxygen.

The term “vaccine” as used herein, includes a composition (e.g., a suspension) of antigens or cells, preferably attenuated cells or organisms, which produces or elicits an immune response (e.g., produces or elicits active immunity) when administered to a subject. The term “vaccine” also includes DNA vaccines in which the nucleic acid molecule encoding an antigen or antigenic portion thereof in a pharmaceutical composition is administered to a subject. For genetic immunization, suitable delivery methods known to those skilled in the art include direct injection of plasmid DNA into muscles (Wolff et al., Hum. Mol. Genet. 1:363, 1992), delivery of DNA complexed with specific protein carriers (Wu et al., J. Biol. Chem. 264:16985, 1989), coprecipitation of DNA with calcium phosphate (Benvenisty et al., Proc. Natl. Acad. Sci. U.S.A. 83:9551, 1986), encapsulation of DNA in liposomes (Kaneda et al., Science 243:375, 1989), particle bombardment (Tang et al., Nature 356:152, 1992 and Eisenbraun et al., DNA Cell Biol. 12:791, 1993), and in vivo infection using cloned retroviral vectors (Seeger et al., Proc. Natl. Acad. Sci. U.S.A. 81:5849, 1984)

The vaccine to be administered can comprise an antigen. The term “antigen” includes agents which provoke an immune response independently as well as those which provoke an immune response when incorporated in to a vaccine of the invention. The term “antigen epitope” includes fragments of proteins capable of determining antigenicity. An epitope may comprise, for example, a peptide of six to eight residues in length. Some epitopes may be significantly larger.

In certain embodiments, the vaccine comprises an antigenic polypeptide or an antigenic fragment thereof. The polypeptide can be a recombinant polypeptide or can be isolated from a cell or organism. In other situations, the vaccine comprises a nucleic acid encoding an antigen or antigenic fragment thereof. In other situations, the vaccine comprises a whole organism, e.g., a live, heat killed, or chemically attenuated virus, bacterium, mycoplasma, fungus, or protozoan.

For example, antigens include proteins and other molecules which are specifically associated with surfaces of particular types of cancer cells, e.g. tumor cells. Many forms of cancer can be characterized by production of proteins associated with that form of the disease, and are not found in normal tissue. Often these proteins are used at a specific stage of embryonic development, and are not observed during normal adult lifetime. These antigens are particularly useful as a source of epitopes for anticancer vaccines.

In other embodiments, the vaccines useful in the practice invention may be derived from antigens or extracts associated with the surfaces or secretion products of micro-organisms or pathogens. The term “pathogen” is meant to include organisms that cause disorders, such disorders produced by one or more particular species of bacteria, viruses, fungi, and protozoans which are disease-producing organisms. Examples of pathogens include gram-negative bacterial species such as Escherichia coli serotype 0157:H7, Helicobacter pylori, H. mustelae, Haemophilus influenzae and H. ducreyi, Pseudomonas aeruginosa, Shigella dysenteria, Salmonella typhi and S. paratyphi; Gram-positive bacterial species such as Mycobacterium tuberculosis, M. leprae, Clostridium tetani, Staphylococcus aureus, and Streptococcus hemolyticus; obligate intracellular bacterial organisms such as Rickettsia and Chlamydia species; retroviruses, which are RNA containing viruses that use reverse transcriptase to synthesize complementary DNA, including but not limited to HIV-1, and -2; other pathogenic viruses such HSV-I and -II, non-A non-B non-C hepatitis virus, pox viruses, and rabies viruses; fungi such as Candida and Aspergillus species; protozoa such as Cryptosporidium parvum, Entamoeba histolytica and Giardia lamblia; and animal pathogens such as Newcastle disease virus. Obtaining unique epitopes from these organisms by screening proteins and by assaying peptides in vitro are commonly known to those skilled in the art; many examples have been described and the appropriate amino acid residue sequence may be accessed from Genbank.

The antigen may be pharmacologically active for treating a disease, e.g., smallpox, yellow fever, distemper, cholera, fowl pox, scarlet fever, diphtheria, tetanus, whooping cough, rabies, mumps, measles, foot and mouth disease, poliomyelitis, severe acute respiratory syndrome (SARS), HIV, herpes simplex virus 1 (HSV1), herpes simples virus 2 (HSV2), varicella zoster virus (herpes zoster), variola virus, hepatitis virus (e.g., A, B, or C), cytomegalovirus, Epstein Barr, papilloma virus, viral influenza (e.g., avian influenza, e.g., the H5N1 strain of avian influenza), viral parainfluenza, adenovirus, viral encephalitis, viral meningitis, arbovirus, arenavirus, picornavirus, coronavirus, or syncytial virus. In some situations, the antigen is effective against a newly emergent virus, e.g., a vector of microbial bioterrorism (see, e.g., Harrison's Principles of Internal Medicine, 14th Edition, McGraw-Hill, 1998).

The antigen may be an antigen that ordinarily evokes a weak immune response. The methods described herein may be used to strengthen the immune response to an antigen with otherwise low immunogenicity. An antigen with low immunogenicity may produce resistance to the pathogen of interest in less than 10%, 20% 30%, 40%, 50%, 60%, 70%, 80%, 90%, or 95% of patients.

The vaccine can be administered alone or in combination with other antigens, using methods and materials known to those skilled in the art for vaccines. The immunological response may be used therapeutically or prophylactically and may provide antibody immunity or cellular immunity, such as that produced by T lymphocytes.

The antigen may be conjugated to a carrier molecule. Suitable immunogenic carriers include proteins, polypeptides or peptides such as albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular mass of at least 1,000 Daltons, preferably greater than 10,000 Daltons. Carrier molecules often contain a reactive group to facilitate covalent conjugation to the hapten. The carboxylic acid group or amine group of amino acids or the sugar groups of glycoproteins are often used in this manner. Carriers lacking such groups can often be reacted with an appropriate chemical to produce them. Preferably, an immune response is produced when the immunogen is injected into animals such as mice, rabbits, rats, goats, sheep, guinea pigs, chickens, and other animals, most preferably mice and rabbits. Alternatively, a multiple antigenic peptide comprising multiple copies of the protein or polypeptide, or an antigenically or immunologically equivalent polypeptide may be sufficiently antigenic to improve immunogenicity without the use of a carrier.

The antigen may be administered with an adjuvant. Adjuvants can be broadly separated into two classes, based on their principal mechanisms of action: vaccine delivery systems and immunostimulatory adjuvants (see, e.g., Singh et al., Curr. HIV Res. 1:309-20, 2003). Vaccine delivery systems are generally particulate formulations, e.g., emulsions, microparticles, Immune-stimulating complexes (I SCOMs), and liposomes, and can target associated antigens into antigen presenting cells (APC). In contrast, immunostimulatory adjuvants are predominantly derived from pathogens and often represent pathogen associated molecular patterns (PAMP), e.g., LPS, MPL, or CpG DNA, which activate cells of the innate immune system. Other adjuvants known in the art include, TiterMax SuperCarrier, L-tyrosine, Montanide, AdjuPrime, Nitrocellulose-absorbed protein, and Gerbu adjuvant. Certain adjuvants are appropriate for human patients, non-human animals, or both. In some situations, oxygen administered in the methods described herein does not directly immunostimulate, but is immunostimulating based on its ability to prevent inhibition of an immune response.

As used herein, “adjuvant” or “suitable adjuvant” describes a substance capable of being combined with the antigen to enhance an immune response in a subject without deleterious effect on the subject. A suitable adjuvant can be, but is not limited to, for example, an immunostimulatory cytokine, SYNTEX adjuvant formulation 1 (SAF-1) composed of 5% (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion. The adjuvant, such as an immunostimulatory cytokine, can be administered before the administration of the antigen, concurrent with the administration of the antigen or up to five days after the administration of the antigen to a subject. QS-21, similarly to alum, complete Freund's adjuvant, SAF, etc., can be administered simultaneously with or within hours of administration of the antigen.

B. Exemplary Vaccines Against Infectious Agents

Infectious diseases that can be treated with the subject vaccine combinations include those caused by infectious agents such as, but not limited to, viruses, bacteria, fungi protozoa, helminths, and parasites.

Examples of viruses that have been found in humans include, but are not limited to, Retroviridae (e.g., human immunodeficiency viruses, such as HIV-1 (also referred to as HTLV-III, LAV or HTLV-III/LAV, or HIV-III; and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (e.g., hemorrhagic fever viruses); Reoviridae (e.g., reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted, e.g., Hepatitis C); Norwalk and related viruses, and astroviruses.

Retroviruses that results in infectious diseases in animals and humans include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigenic or immunogenic in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses), the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigenic or immunogenic in vertebrate animals include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus), the Beta-herpesviruses (Human cytomegalovirus and cytomegaloviruses of swine, monkeys and rodents), the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species), the genus Aviadenovirus (Avian adenoviruses), and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents.

Bacterial infections or diseases that can be treated by methods of the present invention are caused by bacteria including, but not limited to, bacteria that have an intracellular stage in its life cycle, such as mycobacteria (e.g., Mycobacteria tuberculosis, Mycobacteria bovis, Mycobacteria avium, Mycobacteria leprae, or Mycobacteria africanum), rickettsia, mycoplasma, chlamydia, and legionella. Other examples of bacterial infections contemplated include, but are not limited to, infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, Neisseria species. Specific examples of infectious bacteria include, but are not limited to: Helicobacter pyloris, Borelia burgdorferi, Legionella pneumophilia, Mycobacteria tuberculosis, Mycobacteria avium, Mycobacteria intracellulare, Mycobacteria kansaii, Mycobacteria gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Fungal diseases that can be treated by methods of the present invention include, but are not limited to, aspergilliosis, crytococcosis, sporotrichosis, coccidioidomycosis, paracoccidioidomycosis, histoplasmosis, blastomycosis, zygomycosis, and candidiasis.

Parasitic diseases that can be treated by methods of the present invention include, but are not limited to, amebiasis, malaria, leishmania, coccidia, giardiasis, cryptosporidiosis, toxoplasmosis, and trypanosomiasis. Also encompassed are infections by various worms such as, but not limited to, ascariasis, ancylostomiasis, trichuriasis, strongyloidiasis, toxoccariasis, trichinosis, onchocerciasis, filaria, and dirofilariasis. Also encompassed are infections by various flukes such as, but not limited to, schistosomiasis, paragonimiasis, and clonorchiasis. Parasites that cause these diseases can be classified based on whether they are intracellular or extracellular. An “intracellular parasite,” as used herein, is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite,” as used herein, is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites.” These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp.

The term “antipathogenic extract” includes an extract from a pathogen or microorganism which contains antigens which can be used in the methods of the invention to make antipathogenic vaccines. In one embodiment, the antipathogenic extract includes surface proteins or secretion products of the pathogen.

C. Additional Adjuvants

The tumor vaccines of the present invention may contain an adjuvant that induces non-specific immune responses. The adjuvant can be used alone or in combination of two or more kinds. As the adjuvant, examples include Freund complete adjuvant, Freund incomplete adjuvant, bacterial preparations such as BCG, bacterial component preparations such as tuberculin, natural macromolecular substances such as keyhole limpet hemocyanine and yeast mannan, Alum, synthetic adjuvant preparations such as Titer Max Gold and the like. However, the adjuvants are not limited to these specific examples, and any substances may be used so far that they are effective as adjuvants. Whether an adjuvant should be used or not can be judged by intensity of inflammatory reaction at a site of administration or intensity of antitumor effect induced as a result of the administration as a standard. For example, alternate administrations of the tumor vaccine containing an adjuvant and the vaccine without adjuvant can be applied to the same site. Optionally, an adjuvant may be administered with the first dose of vaccine and not with subsequent doses (i.e. booster shots). In an alternative embodiment, a strong adjuvant may be administered with the first dose of vaccine and a weaker adjuvant or lower dose of the strong adjuvant may be administered with subsequent doses.

### VI. Combination Therapy

All of the methods of the inventions described herein may include the administration of oxygen to a subject in combination with a therapeutically effective amount of a therapeutic agent. As used herein, “therapeutically effective amount” means an amount sufficient to effect beneficial or desired clinical results. An effective amount can be administered in one or more administrations. In terms of treatment of a tumor, an “effective amount” of oxygen and/or a therapeutic agent is an amount sufficient to palliate, ameliorate, stabilize, reverse, slow, or delay progression of a tumor in accordance with clinically acceptable standards for treatment of tumors. Detection and measurement of indicators of efficacy can be measured by a number of available diagnostic tools, including, but not limited to, for example, by physical examination including blood tests, urinalysis, X-rays, CT scan, and biopsy.

The therapeutic agent administered may be an oxygen-enhancing substance. As used herein, an “oxygen-enhancing substance” is a compound, drug, or natural or synthetic blood product that increases local oxygen tension in a tissue. As used herein, “local oxygen tension” refers to the concentration of oxygen in local tissue microenvironment.

Oxygen-enhancing substances are known in the art and include, but not limited to, perfluorocarbon based oxygen delivery drugs (e.g., RSR13, an analog of the drugs clofibrate and bezofibrate (Allos Therapeutics, Denver, Colo.; see also Wahr et al., Anesth. Analg. 92:615-620, 2001)); drugs based on haemoglobin molecules coated with polyethylene glycol (e.g., MP4; see Wettstein et al., Crit. Care Med. 31:1824-1830, 2003); Hemolink™ (Hemosol Corp., Ontario, Canada); Hemopure™ (Biopure Corp., Cambridge, Mass.); PolyHeme™ (Northfield Laboratories, Evanston, Ill.); Oxygent™ (Alliance Pharmaceutical Corp., San Diego, Calif.); Oxycyte™ (Synthetic Blood International, Costa Mesa, Calif.); PHER-O2 (Sanguine Corp., Pasadena, Calif.); Albrec (Mitsubishi Pharma, Osaka, Japan); Advate (Baxter, Deerfield, Ill.); and Synthocytes™ (Andaris Group Ltd, Nottingham, UK). A physician treating a subject would readily appreciate how to use these substances in the methods described herein.

The therapeutic agent may be an adenosine pathway antagonist. The adenosine pathway antagonist can be an adenosine receptor antagonist, such as an adenosine analog or other small organic molecule, that binds to an adenosine receptor and inhibits (partially or completely) the ability of adenosine to induce a receptor-dependent signal. The adenosine pathway antagonist can also be an agent that inhibits the biosynthesis of adenosine or otherwise reduces adenosine levels, inhibits expression of one or more adenosine receptors, and/or desensitizes or inhibits adenosine receptor-mediated signaling. As an example of the later, the invention contemplates the use of cAMP inhibitors as adenosine pathway antagonists to be used to improve vaccinations.

For example, the adenosine pathway antagonist may be a chemical compound that binds or interacts with an adenosine receptor, e.g., the A2a or A2b adenosine receptor. The antagonist may be a peptide, or a peptidomimetic, that binds the adenosine receptor. Exemplary antagonists that can be used in the methods described herein are described in U.S. Pat. Nos. 5,565,566, 5, 545, 627, 5,981,524, 5,861,405, 6,066,642, 6,326,390, 5,670,501, 6,117,998, 6,232,297, 5,786,360, 5,424,297, 6,313,131, 5,504,090, and 6,322,771, all of which are incorporated herein by reference. Other nonlimiting examples include ZM241385 (4-(2-[7-amino-2-(2-furyl[1,2,4]-triazolo[2,3-α[1,3,5]triazin-5-yl-aminoethyl)phenol, Tocris Cookson Inc., Ellisville, Mo.), 1,3,7, trimethylxanthine (caffeine), theophilline, teobromin, SCH5826 (5-amino-7-(2-phenylethyl)-2-(2-furyl)-pyrazolo[4,3-E]-1,2,4-triazolo[1,5-c]pyrimidine), enprofylline (Sigma-Aldrich, Steinheim, Germany), and KW-6002 (Istradefylline, Kyowa Pharmaceutical, Princeton, N.J.).

Specific examples of small molecule A2AR antagonists are provided below. One especially preferred group of A2AR antagonists is disclosed in WO/2002/055524. This group includes thieno (3,2-d) pyrimidines and furano (3,2-d) pyrimidines. Molecules of this group may be represented by formula I:

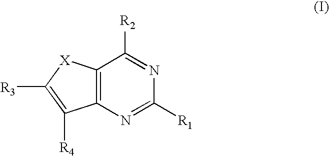

wherein X is S or O;

Ri is selected from H, alkyl, aryl, hydroxy, alkoxy, aryloxy, thioalkyl, thioaryl, halogen, CN, COR5, CO2R5, CONR6R7, CONR5NR6R7, NR6R7, NRsCONR6R7, NR5COR6, NR5CO2R8, and NR5SO2R8;

R2 is selected from aryl attached via an unsaturated carbon atom;

R3 is selected from H, alkyl, hydroxy, alkoxy, halogen, CN and NO2;

R4 is selected from H, alkyl, aryl, hydroxy, alkoxy, aryloxy, thioalkyl, thioaryl, halogen, CN, N02, COR5, C02R5, CONR6R7, CONR5NR6R7, NR6R7, NR5CONR6R7, NR5COR6, NR5CO2R8 and NR5S02R8;

R5, R6 and R7 are independently selected from H, alkyl and aryl, or where R6 and R7 are in an (NR6R7) group, R6 and R7 may be linked to form a heterocyclic group, or where R5, R6 and R7 are in a (CONR5NR6R7) group, R5 and R6 may be linked to form a heterocyclic group;

and R8 is selected from alkyl and aryl, or a pharmaceutically acceptable salt thereof or prodrug thereof.

Examples of such molecules are laid out in Table 1:

TABLE 1

| Example | Structure | Compound Name |
|---|---|---|
| 1 (A) | 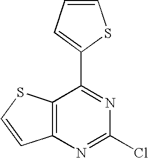 | 2-chloro-4-(2-thienyl)thieno[3,2-d]pyrimidine |
| 2 (E) | 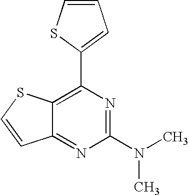 | N,N-dimethyl-4-(2-thienyl)thieno[3,2-d]pyrimidine- 2-amine |
| 3 (A) | 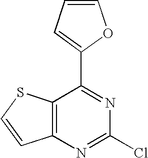 | 2-chloro-4-(2-furyl)thieno[3,2-d]pyrimidine |
| 4 (E) | 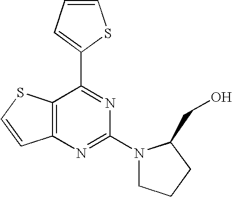 | (2R)-2-(2-hydroxymethylpyrrolidin-1-yl)-4-(2- thienyl)thieno[3,2-d]pyrimidine |
| 5 (E) | 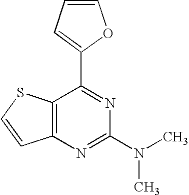 | N,N-dimethyl-4-(2-furyl)thieno[3,2-d]pyrimidine-2- amine |
| 6 (E) | 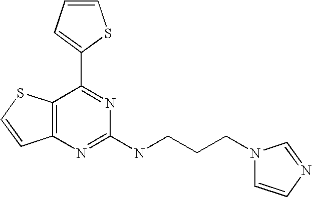 | N-(3-(1H-imidazol-1-yl)propyl)-4-(2- thienyl)thieno[3,2-d]pyrimidine-2-amine |
| 7 (E) | 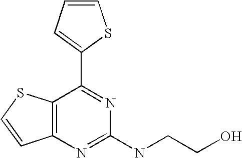 | N-(2-hydroxyethyl)-4-(2-thienyl)thieno[3,2- d]pyrimidine-2-amine |
| 8 (E) | 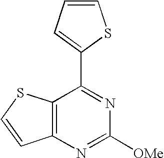 | 2-methoxy-4-(2-thienyl)thieno[3,2-d]pyrimidine |
| 9 (B) | 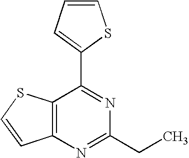 | 2-ethyl-4-(2-thienyl)thieno[3,2-d]pyrimidine |
| 10 (E) | 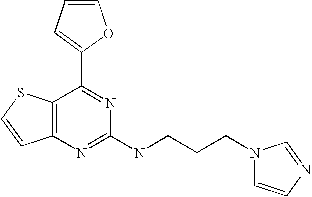 | N-(3-(1H-imidazol-1-yl)propyl)-4-(2- furyl)thieno[3,2-d]pyrimidine-2-amine |
| 11 (A) | 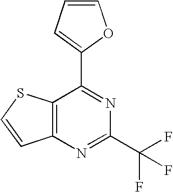 | 4-(2-furyl)-2-trifluoromethylthieno[3,2-d]pyrimidine |
| 12 (A) | 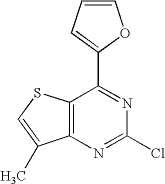 | 2-chloro-4-(2-furyl)-7-methylthieno(3,2- d]pyrimidine |
| 13 (A) | 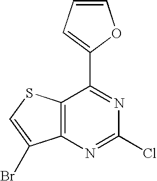 | 7-bromo-2-chloro-4-(2-furyl)thieno[3,2- d]pyrimidine |
| 14 (E) | 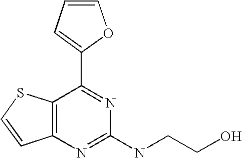 | 4-(2-furyl)-N-(2-hydroxyethyl)thieno[3,2- d]pyrimidine-2-amine |
| 15 (E) | 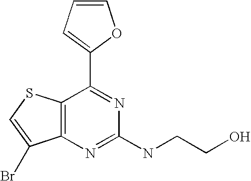 | 7-bromo-4-(2-furyl)-N-(2-hydroxyethyl)thieno[3,2- d]pyrimidine-2-amine |
| 16 (E) | 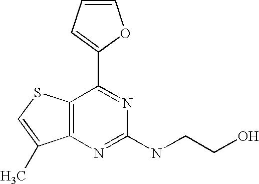 | 4-(2-furyl)-N-(2-hydroxyethyl)-7-methylthieno[3,2- d]pyrimidine-2-amine |
| 17 (A) | 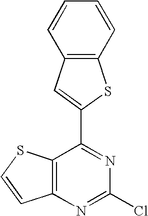 | 4-(2-benzothiophenyl)-2-chlorothieno[3,2- d]pyrimidine |
| 18 (A) | 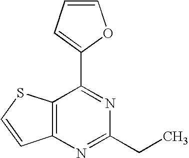 | 2-ethyl-4-(2-furyl)thieno[3,2-d]pyrimidine |
| 19 (E) | 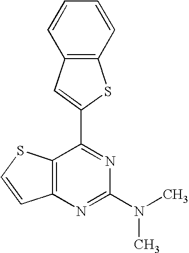 | 4-(2-benzothiophenyl)-N,N-dimethylthieno[3,2- d]pyrimidine-2-amine |
| 20 (E) | 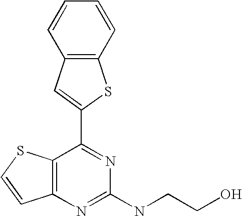 | 4-(2-benzothiophenyl)-N-(2- hydroxyethyl)thieno[3,2-d]pyrimidine-2-amine |
| 21 (E) | 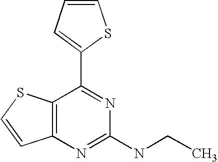 | N-ethyl-4-(2-thienyl)thieno[3,2-d]pyrimidine-2- amine |
| 22 (E) | 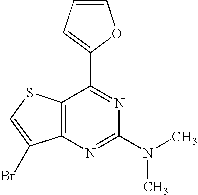 | 7-bromo-N,N-dimethyl-4-(2-furyl)thieno[3,2- d]pyrimidine-2-amine |
| 23 (E) | 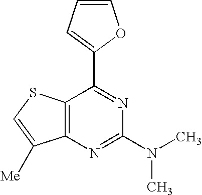 | 4-(2-furyl)-7,N,N-trimethylthieno[3,2-d]pyrimidine- 2-amine |
| 24 (A) | 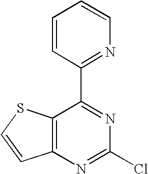 | 2-chloro-4-(2-pyridyl)thieno[3,2-d]pyrimidine |
| 25 (E) | 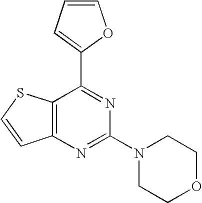 | 4-(2-furyl)-2-morpholinothieno[3,2-d]pyrimidine |
| 26 (E) | 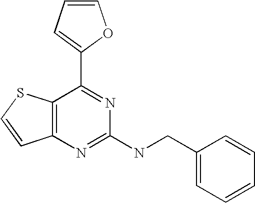 | N-benzyl-4-(2-furyl)thieno[3,2-d]pyrimidine-2- amine |
| 27 (E) | 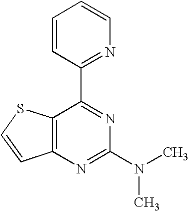 | N,N-dimethyl-4-(2-pyridyl)thieno[3,2-d]pyrimidine- 2-amine |
| 28 (B) | 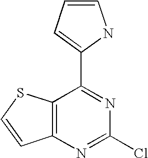 | 2-chloro-4-(1H-pyrrol-1-yl)thieno[3,2-d]pyrimidine |
| 29 (A) | 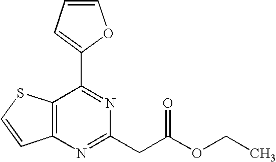 | Ethyl 4-(2-furyl)thieno[3,2-d]pyrimidine-2-acetate |
| 30 (A) | 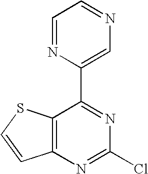 | 2-chloro-4-(2-pyrazinyl)thieno[3,2-d]pyrimidine |
| 31 (P) | 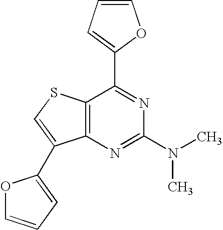 | 4,7-bis(2-furyl)-N,N-dimethylthieno[3,2- d]pyrimidine-2-amine |
| 32 (E) | 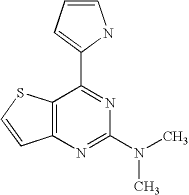 | N,N-dimethyl-4-(1H-pyrrol-1-yl)thieno[3,2- d]pyrimidine-2-amine |
| 33 (E) | 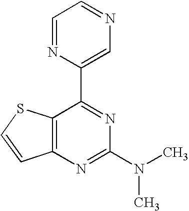 | N,N-dimethyl-4-(2-pyrazinyl)thieno[3,2- d]pyrimidine-2-amine |
| 34 (E) | 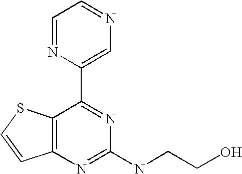 | N-(2-hydroxyethyl)-4-(2-pyrazinyl)thieno[3,2- d]pyrimidine-2-amine |
| 35 (E) | 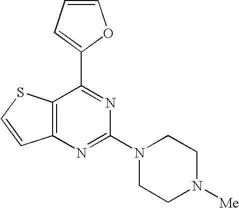 | 4-(2-furyl)-2-(4-methylpiperazinyl)thieno[3,2- d]pyrimidine |
| 36 (E) | 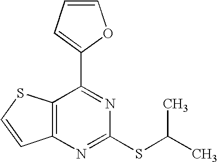 | 4-(2-furyl)-2-isopropylthiothieno[3,2-d]pyrimidine |
| 37 (E) | 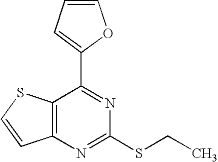 | 2-ethylthio-4-(2-furyl)thieno[3,2-d]pyrimidine |
| 38 (E) | 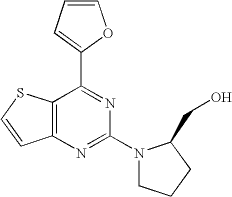 | (2R)-4-(2-furyl)-2-(2-hydroxymethylpyrrolidin-1- yl)thieno[3,2-d]pyrimidine |
| 39 (E) | 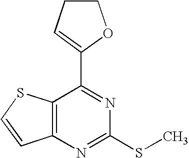 | 4-(2-furyl)-2-methylthiothieno[3,2-d]pyrimidine |
| 40 (E) | 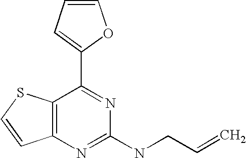 | N-allyl-4-(2-furyl)thieno[3,2-d]pyrimidine-2-amine |
| 41 (A) | 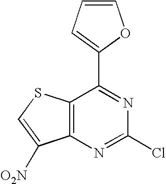 | 2-chloro-4-(2-furyl)-7-nitrothieno[3,2-d]pyrimidine |
| 42 (E) | 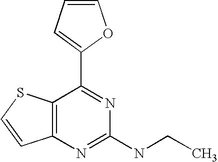 | N-ethyl-4-(2-furyl)thieno[3,2-d]pyrimidine-2-amine |
| 43 (E) | 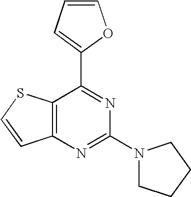 | 4-(2-furyl)-2-(pyrrolidin-1-yl)thieno[3,2- d]pyrimidine |
| 44 (E) | 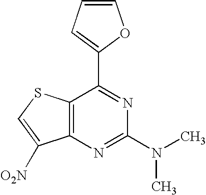 | N,N-dimethyl-4-(2-furyl)-7-nitrothieno[3,2- d]pyrimidine-2-amine |
| 45 (E) | 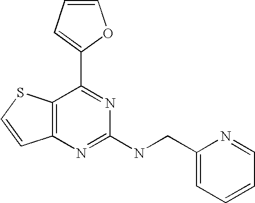 | 4-(2-furyl)-N-(2-pyridylmethyl)thieno[3,2- d]pyrimidine-2-amine |
| 46 (A) | 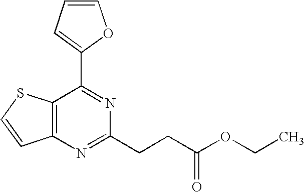 | Ethyl 3-(4-(2-furyl)thieno[3,2-d]pyrimidine-2- yl)propionate |
| 47 (E) | 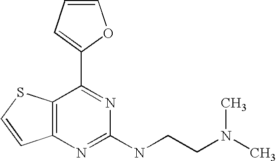 | N-(2-dimethylaminoethyl)4-(2-furyl)thieno[3,2- d]pyrimidine-2-amine |
| 48 (K) | 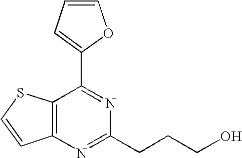 | 3-(4-(2-furyl)thieno[3,2-d]pyrimidin-2-yl)propanol |
| 49 (M) | 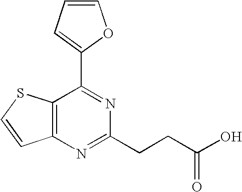 | 3-(4-(2-furyl)thieno[3,2-d]pyrimidine-2-yl)propionic acid |
| 50 (N) | 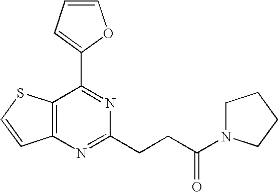 | 4-(2-furyl)-2-(3-oxo-3-(1- pyrrolidinyl)propyl)thieno[3,2-d]pyrimidine |
| 51 (J) | 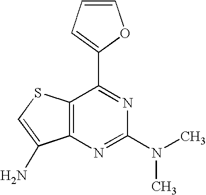 | 7-amino-N,N-dimethyl-4-(2-furyl)thieno[3,2- d]pyrimidine-2-amine |
| 52 (C) | 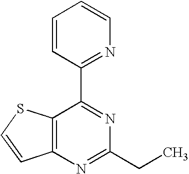 | 2-ethyl-4-(2-pyridyl)thieno[3,2-d]pyrimidine |
| 53 (E) | 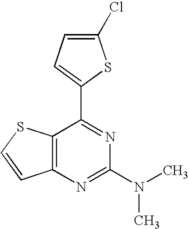 | 4-(5-chloro-2-thienyl)-N,N-dimethylthieno[3,2- d]pyrimidine-2-amine |
| 54 (K) | 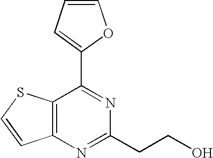 | 2-(4-(2-furyl)thieno[3,2-d]pyrimidin-2-yl)ethanol |
| 55 (I) | 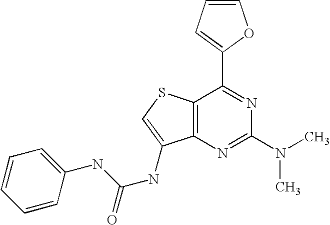 | N-(2-dimethylamino-4-(2-furyl)thieno[3,2- d]pyrimidine-7-yl)-N′-phenylurea |
| 56 (G) | 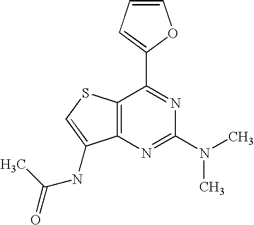 | N-(2-dimethylamino-4-(2-furyl)thieno[3,2- d]pyrimidine-7-yl)acetamide |
| 57 (G) | 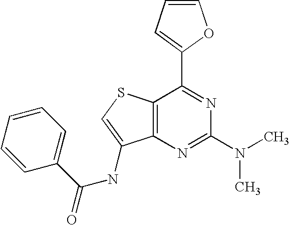 | N-(2-dimethylamino-4-(2-furyl)thieno[3,2- d]pyrimidine-7-yl)benzamide |
| 58 (E) | 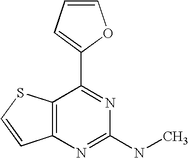 | 4-(2-furyl)-N-methylthieno[3,2-d]pyrimidine-2- amine |
| 59 (G) | 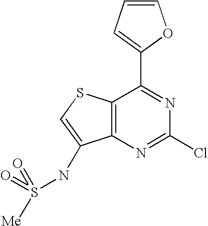 | N-(2-chloro-4-(2-furyl)thieno[3,2-d]pyrimidine-7- yl)methanesulphonamide |
| 60 (G) | 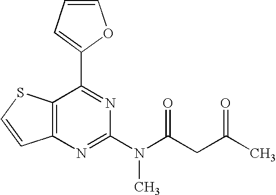 | N-(4-(2-furyl)thieno[3,2-d]pyrimidine-2-yl)-N- methyl-3-oxobutanamide |
| 61 (E) | 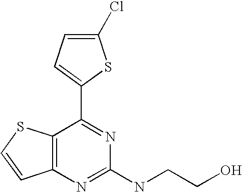 | 4-(5-chloro-2-thienyl)-N-(2- hydroxyethyl)thieno[3,2-d]pyrimidine-2-amine |
| 62 (C) | 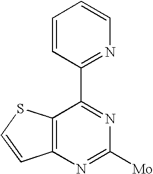 | 2-methyl-4-(2-pyridyl)thieno[3,2-d]pyrimidine |
| 63 (C) | 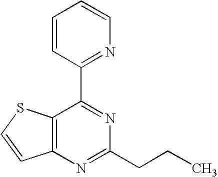 | 2-n-propyl-4-(2-pyridyl)thieno[3,2-d]pyrimidine |
| 64 (C) | 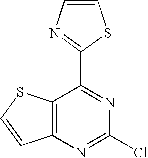 | 2-chloro-4-(2-thiazolyl)thieno[3,2-d]pyrimidine |
| 65 (E) | 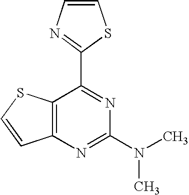 | N,N-dimethyl-4-(2-thiazolyl)thieno[3,2- d]pyrimidine-2-amine |
| 66 (C) | 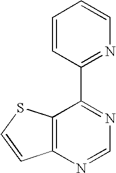 | 4-(2-pyridyl)thieno[3,2-d]pyrimidine |
| 67 (E) | 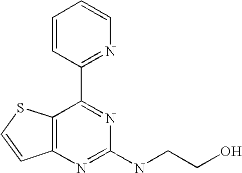 | N-(2-hydroxyethyl)-4-(2-pyridyl)thieno[3,2- d]pyrimidine-2-amine |
| 68 (E) | 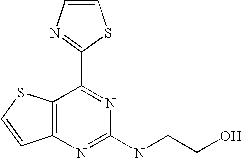 | N-(2-hydroxyethyl)-4-(2-thiazolyl)thieno[3,2- d]pyrimidine-2-amine |
| 69 (L) | 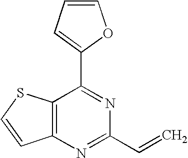 | 4-(2-furyl)-2-vinylthieno[3,2-d]pyrimidine |
| 70 (C) | 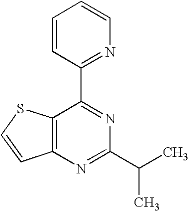 | 2-isopropyl-4-(2-pyridyl)thieno[3,2-d]pyrimidine |
| 71 (E) | 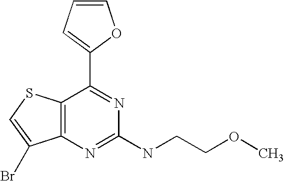 | N-(2-methoxyethyl)-4-(2-furyl)thieno[3,2- d]pyrimidine-2-amine |
| 72 (E) | 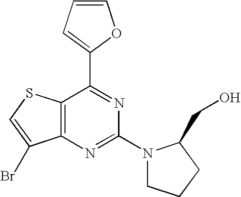 | (2R)-7-bromo-4-(2-furyl)-2-(2- hydroxymethylpyrrolidin-1-yl)thieno[3,2- d]pyrimidine |
| 73 (A) | 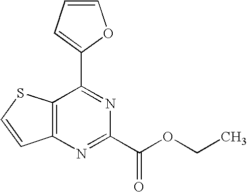 | Ethyl 4-(2-furyl)thieno[3,2-d]pyrimidine-2- carboxylate |
| 74 (E) | 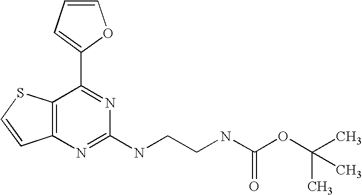 | tert-butyl (2-(4-(2-furyl)thieno[3,2-d]pyrimidine-2- ylamino)ethyl)carbamate |
| 75 (F) | 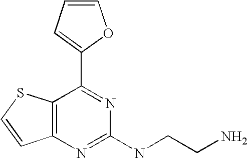 | N-(2-aminoethyl)-4-(2-furyl)thieno[3,2- d]pyrimidine-2-amine |
| 76 (E) | 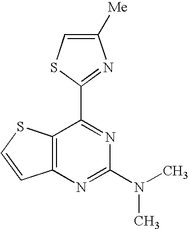 | N,N-dimethyl-4-(4-methyl-2-thiazolyl)thieno[3,2- d]pyrimidine-2-amine |
| 77 (H) | 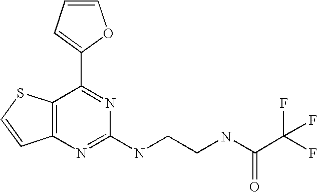 | N-(2-(4-(2-furyl)thieno[3,2-d]pyrimidine-2- ylamino)ethyl)trifluoroacetamide |
| 78 (E) | 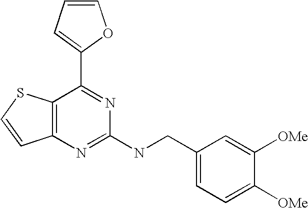 | N-(3,4-dmethoxybenzyl)-4-(2-furyl)thieno[3,2- d]pyrimidine-2-amine |
| 79 (F) | 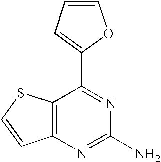 | 4-(2-furyl)thieno[3,2-d]pyrimidine-2-amine |
| 80 (C) | 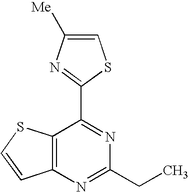 | 2-ethyl-4-(4-methyl-2-thiazolyl)thieno[3,2- d]pyrimidine |
| 81 (K) | 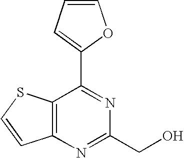 | 4-(2-furyl)thieno[3,2-d]pyrimidine-2-methanol |
| 82 (C) | 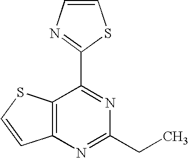 | 2-ethyl-4-(2-thiazolyl)thieno[3,2-d]pyrimidine |
| 83 (H) | 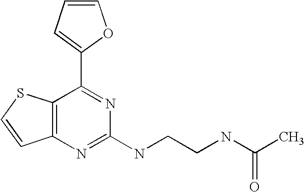 | N-(2-(4-(2-furyl)thieno[3,2-d]pyrimidine-2- ylamino)ethyl)acetamide |
| 84 (H) | 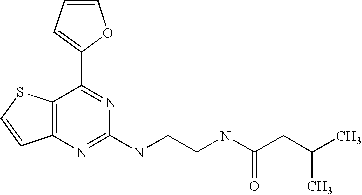 | N-(2-(4-(2-furyl)thieno[3,2-d]pyrimidine-2- ylamino)ethyl)-3-methylbutanamide |
| 85 (H) | 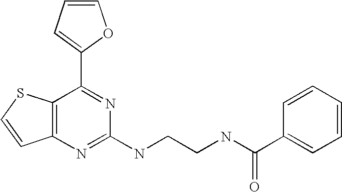 | N-(2-(4-(2-furyl)thieno[3,2-d]pyrimidine-2- ylamino)ethyl)benzamide |
| 86 (H) | 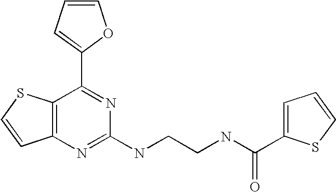 | N-(2-(4-(2-furyl)thieno[3,2-d]pyrimidine-2- ylamino)ethyl)thiophene-2-carboxamide |
| 87 (H) | 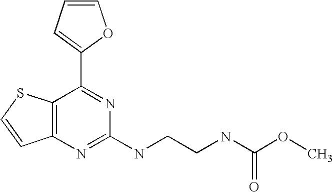 | methyl (2-(4-(2-furyl)thieno[3,2-d]pyrimidine-2- ylamino)ethyl)carbamate |
| 88 (H) | 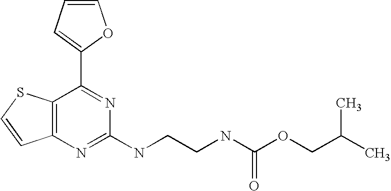 | isobutyl (2-(4-(2-furyl)thieno[3,2-d]pyrimidine-2- ylamino)ethyl)carbamate |
| 89 (H) | 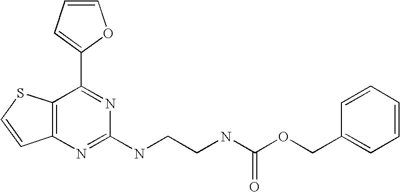 | benzyl (2-(4-(2-furyl)thieno[3,2-d]pyrimidine-2- ylamino)ethyl)carbamate |
| 90 (H) | 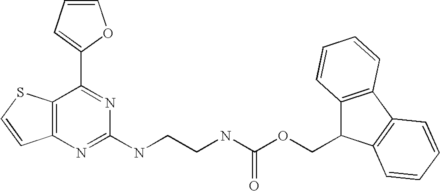 | 9-fluorenylmethyl (2-(4-(2-furyl)thieno[3,2- d]pyrimidine-2-ylamino)ethyl)carbamate |
| 91 (I) | 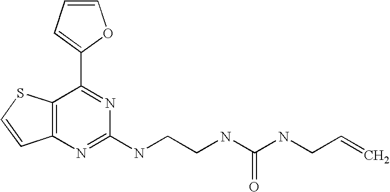 | N-allyl-N′-(2-(4-(2-furyl)thieno[3,2-d]pyrimidine-2- ylamino)ethyl)urea |
| 92 (I) | 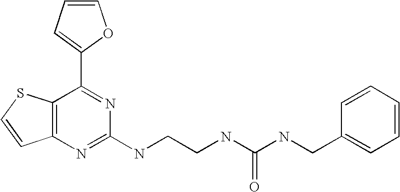 | N-benzyl-N′-(2-(4-(2-furyl)thieno[3,2-d]pyrimidine- 2-ylamino)ethyl)urea |
| 93 (I) | 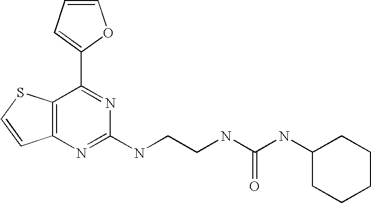 | N-cyclohexyl-N′-(2-(4-(2-furyl)thieno[3,2- d]pyrimidine-2-ylamino)ethyl)urea |
| 94 (I) | 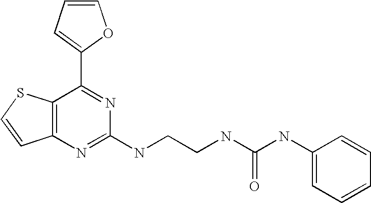 | N-(2-(4-(2-furyl)thieno[3,2-d]pyrimidine-2- ylamino)ethyl)-N′-phenylurea |
| 95 (I) | 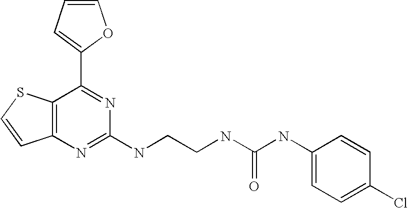 | N-(4-chlorophenyl)-N′-(2-(4-(2-furyl)thieno[3,2- d]pyrimidine-2-ylamino)ethyl)urea |
| 96 (I) | 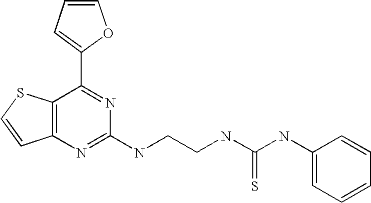 | N-(2-(4-(2-furyl)thieno[3,2-d]pyrimidine-2- ylamino)ethyl)-N′-phenylthiourea |
| 97 (I) | 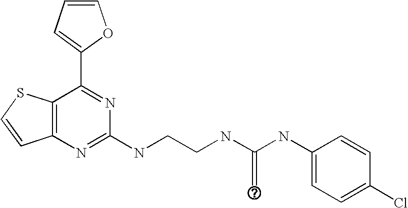 | N-(4-chlorophenyl)-N′-(2-(4-(2-furyl)thieno[3,2- d]pyrimidine-2-ylamino)ethyl)thiourea |
| 98 (H) | 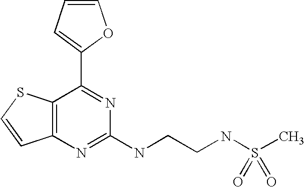 | N-(2-(4-(2-furyl)thieno[3,2-d]pyrimidine-2- ylamino)ethyl)methanesulphonamide |
| 99 (H) | 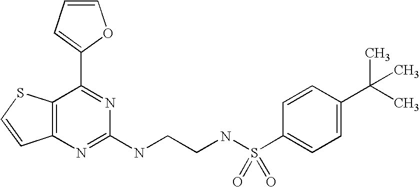 | N-(2-(4-(2-furyl)thieno[3,2-d]pyrimidine-2- ylamino)ethyl)-4-tert-butylphenylsulphonamide |
| 100 (A) | 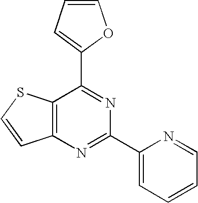 | 4-(2-furyl)-2-(2-pyridyl)thieno[3,2-pyrimidine |
| 101 (G) | 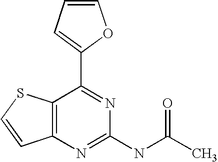 | N-(4-(2-furyl)thieno[3,2-d]pyrimidin-2-yl)acetamide |
| 102 (C) | 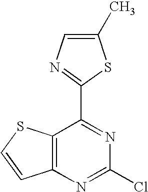 | 2-chloro-4-(5-methyl-2-thiazolyl)thieno[3,2- d]pyrimidine |
| 103 (C) | 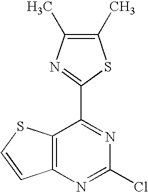 | 2-chloro-4-(4,5-dimethyl-2-thiazolyl)thieno[3,2- d]pyrimidine |
| 104 (E) | 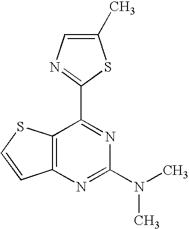 | N,N-dimethyl-4-(5-methyl-2-thiazolyl)thieno[3,2- d]pyrimidine-2-amine |
| 105 (E) | 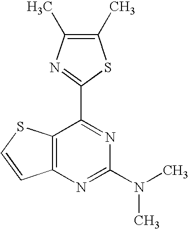 | N,N-dimethyl-4-(4,5-dimethyl-2- thiazolyl)thieno[3,2-d]pyrimidine-2-amine |
| 106 (C) | 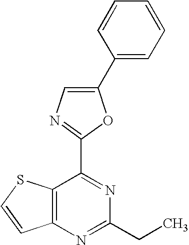 | 2-ethyl-4-(5-phenyl-2-oxazolyl)thieno[3,2- d]pyrimidine |
| 107 (D) | 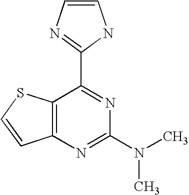 | N,N-dimethyl-4-(1H-imidazol-2-yl)thieno[3,2- d]pyrimidine-2-amine |
| 108 (E) | 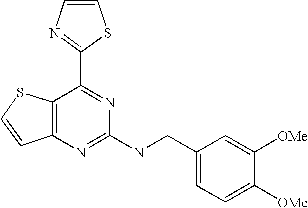 | N-(3,4-dimethoxybenzyl)-4-(2-thiazolyl)thieno[3,2- d]pyrimidine-2-amine |
| 109 (C) | 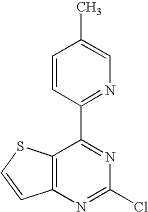 | 2-chloro-4-(5-methyl-2-pyridyl)thieno[3,2- d]pyrimidine |
| 110 (F) | 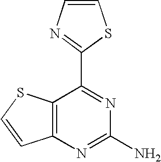 | 4-(2-thiazolyl)thieno[3,2-d]pyrimidine-2-amine |
| 111 (E) | 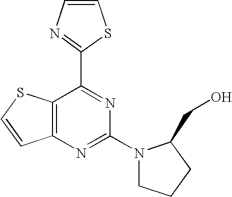 | (2R)-2-(2-hydroxymethylpyrrolidin-1-yl)-4-(2- thiazolyl)thieno[3,2-d]pyrimidine |
| 112 (E) | 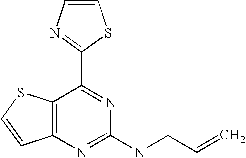 | N-allyl-4-(2-thiazolyl)thieno[3,2-d]pyrimidine-2- amine |
| 113 (C) | 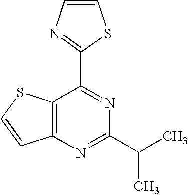 | 2-isopropyl-4-(2-thiazolyl)thieno[3,2-d]pyrimidine |
| 114 (C) | 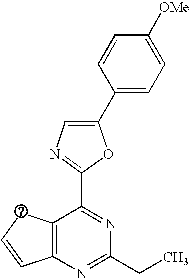 | 2-ethyl-4-(5-(4-methoxyphenyl)-2- oxazolyl)thieno[3,2-d]pyrimidine |
| 115 (E) | 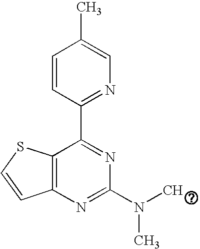 | N,N-dimethyl-4-(5-methyl-2-pyridyl)thieno[3,2- d]pyrimidine-2-amine |
| 116 (G) | 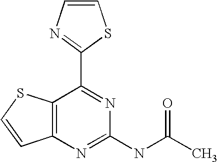 | N-(4-(2-thiazolyl)thieno[3,2-d]pyrimidin-2- yl)acetamide |
| 117 (A) | 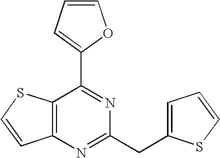 | 4-(2-furyl)-2-(2-thienylmethyl)thieno[3,2- d]pyrimidine |
| 118 (A) | 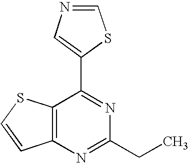 | 2-ethyl-4-(5-thiazolyl)thieno[3,2-d]pyrimidine |
| 119 (A) | 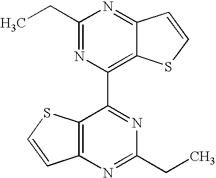 | 2-ethyl-4-(2-ethylthieno[3,2-d]pyrimidin-4- yl)thieno[3,2-d]pyrimidine |
| 120 (D) | 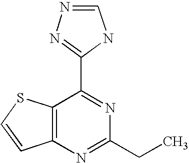 | 2-ethyl-4-(1H-triazol-3-yl)thieno[3,2-d]pyrimidine |
| 121 (D) | 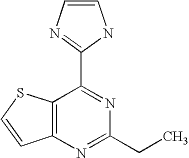 | 2-ethyl-4-(1H-imidazol-2-yl)thieno[3,2-d]pyrimidine |
| 122 (C) | 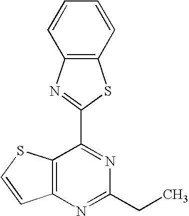 | 4-(2-benzothiazolyl)-2-ethylthieno[3,2-d]pyrimidine |
| 123 (E) | 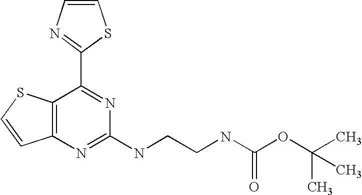 | tert-butyl (2-(4-(2-thiazolyl)thieno[3,2-d]pyrimidine- 2-ylamino)ethyl)carbamate |
| 124 (F) | 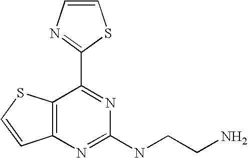 | N-(2-aminoethyl)-4-(2-thiazolyl)thieno[3,2- d]pyrimidine-2-amine |
| 125 (H) | 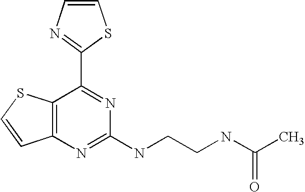 | N-(2-(4-(2-thiazolyl)thieno[3,2-d]pyrimidine-2- ylamino)ethyl)acetamide |
| 126 (I) | 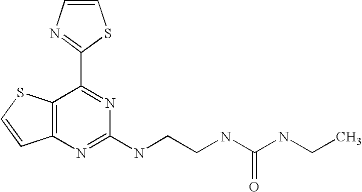 | N-ethyl-N′-(2-(4-(2-thiazolyl)thieno[3,2- d]pyrimidine-2-ylamino)ethyl)urea |
| 127 (I) | 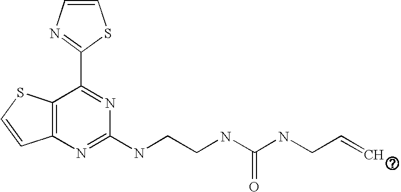 | N-allyl-N′-(2-(4-(2-thiazolyl)thieno[3,2- d]pyrimidine-2-ylamino)ethyl)urea |
| 128 (I) | 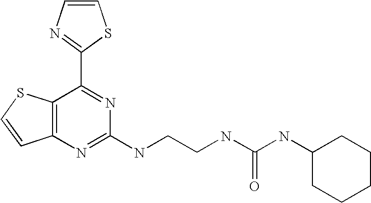 | N-cyclohexyl-N′-(2-(4-(2-thiazolyl)thieno[3,2- d]pyrimidine-2-ylamino)ethyl)urea |
| 129 (H) | 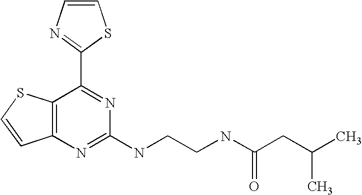 | N-(2-(4-(2-thiazolyl)thieno[3,2-d]pyrimidine-2- ylamino)ethyl)-3-methylbutanamide |
| 130 (H) | 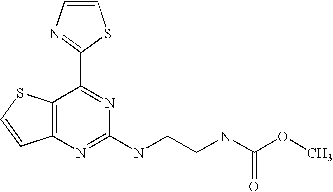 | methyl (2-(4-(2-thiazolyl)thieno[3,2-d]pyrimidine-2- ylamino)ethyl)carbamate |
| 131 (H) | 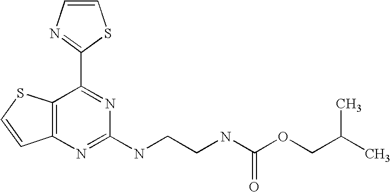 | isobutyl (2-(4-(2-thiazolyl)thieno[3,2-d]pyrimidine- 2-ylamino)ethyl)carbamate |
| 132 (I) | 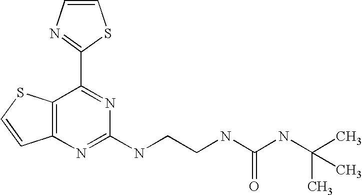 | N-tert-butyl-N′-(2-(4-(2-thiazolyl)thieno[3,2- d]pyrimidine-2-ylamino)ethyl)urea |
| 133 (I) | 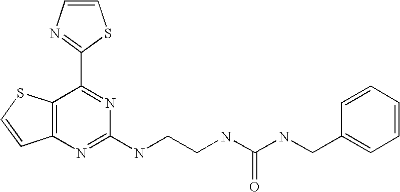 | N-benzyl-N′-(2-(4-(2-thiazolyl)thieno[3,2- d]pyrimidine-2-ylamino)ethyl)urea |
| 134 (I) | 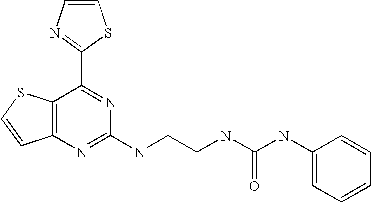 | N-phenyl-N′-(2-(4-(2-thiazolyl)thieno[3,2- d]pyrimidine-2-ylamino)ethyl)urea |
| 135 (I) | 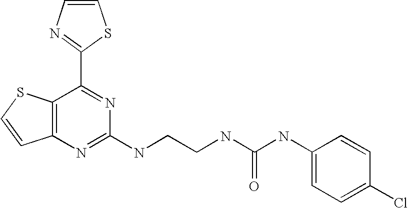 | N-(4-chlorophenyl)-N′-(2-(4-(2-thiazolyl)thieno[3,2- d]pyrimidine-2-ylamino)ethyl)urea |
| 136 (I) | 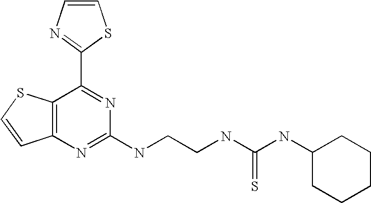 | N-cyclohexyl-N′-(2-(4-(2-thiazolyl)thieno[3,2- d]pyrimidine-2-ylamino)ethyl)thiourea |
| 137 (I) | 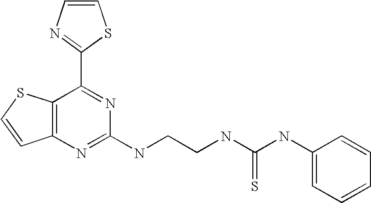 | N-phenyl-N′-(2-(4-(2-thiazolyl)thieno[3,2- d]pyrimidine-2-ylamino)ethyl)thiourea |
| 138 (I) | 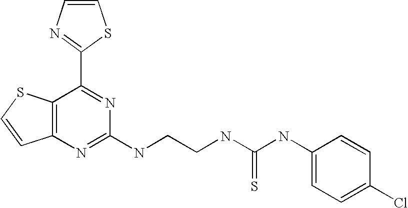 | N-(4-chlorophenyl)-N′-(2-(4-(2-thiazolyl)thieno[3,2- d]pyrimidine-2-ylamino)ethyl)thiourea |
| 139 (C) | 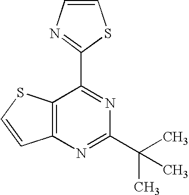 | 2-tert-butyl-4-(2-thiazolyl)thieno[3,2-d]pyrimidine |
| 140 (C) | 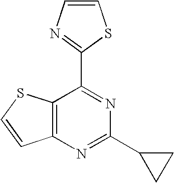 | 2-cyclopropyl-4-(2-thiazolyl)thieno[3,2- d]pyrimidine |
| 141 (C) | 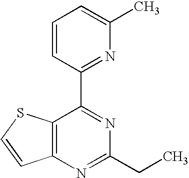 | 2-ethyl-4-(6-methyl-2-pyridyl)thieno[3,2- d]pyrimidine |
| 142 (H) | 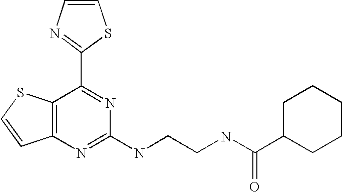 | N-(2-(4-(2-thiazolyl)thieno[3,2-d]pyrimidine-2- ylamino)ethyl)cyclohexylcarboxamide |
| 143 (H) | 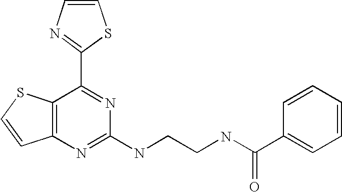 | N-(2-(4-(2-thiazolyl)thieno[3,2-d]pyrimidine-2- ylamino)ethyl)benzamide |
| 144 (H) | 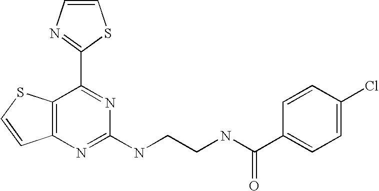 | 4-chloro-N-(2-(4-(2-thiazolyl)thieno[3,2- d]pyrimidine-2-ylamino)ethyl)benzamide |
| 145 (H) | 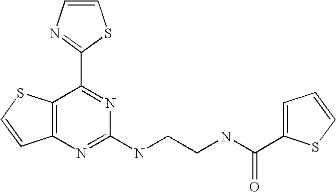 | N-(2-(4-(2-thiazolyl)thieno[3,2-d]pyrimidine-2- ylamino)ethyl)thiophene-2-carboxamide |
| 146 (H) | 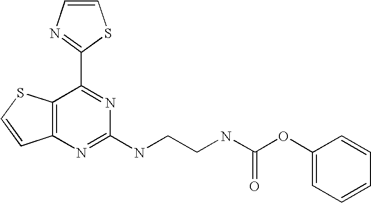 | phenyl (2-(4-(2-thiazolyl)thieno[3,2-d]pyrimidine-2- ylamino)ethyl)carbamate |
| 147 (H) | 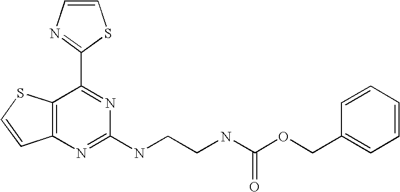 | benzyl (2-(4-(2-thiazolyl)thieno[3,2-d]pyrimidine-2- ylamino)ethyl)carbamate |
| 148 (H) | 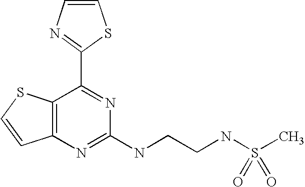 | N-(2-(4-(2-thiazolyl)thieno[3,2-d]pyrimidine-2- ylamino)ethyl)methanesulphonamide |
| 149 (H) | 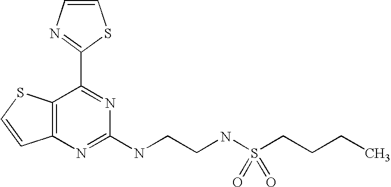 | N-(2-(4-(2-thiazolyl)thieno[3,2-d]pyrimidine-2- ylamino)ethyl)butanesulphonamide |
| 150 (E) | 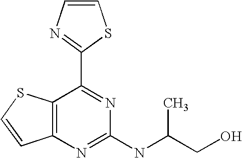 | (1RS)-N-(2-hydroxy-1-methylethyl)-4-(2- thiazolyl)thieno[3,2-d]pyrimidine-2-amine |
| 151 (E) | 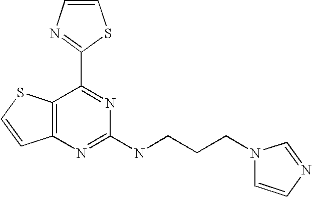 | N-(3-(1H-imidazol-1-yl)propyl)-4-(2- thiazolyl)thieno[3,2-d]pyrimidine-2-amine |
| 152 (E) | 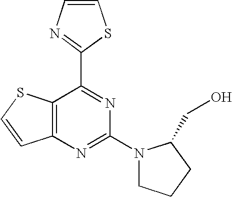 | (2S)-2-(2-hydroxymethylpyrrolidin-1-yl)-4-(2- thiazolyl)thieno[3,2-d]pyrimidine |
| 153 (C) | 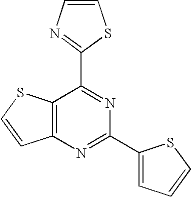 | 4-(2-thiazolyl)-2-(2-thienyl)thieno[3,2-d]pyrimidine |
| 154 (C) | 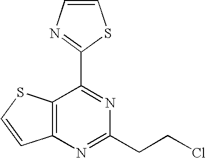 | 2-(2-chloroethyl)-4-(2-thiazolyl)thieno[3,2- d]pyrimidine |
| 155 (O) | 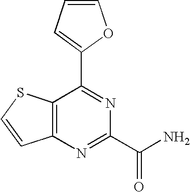 | 4-(2-furyl)thieno[3,2-d]pyrimidine-2-carboxamide |
| 156 (B) | 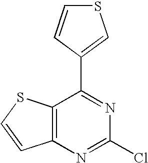 | 2-chloro-4-(3-thienyl)thieno[3,2-d]pyrimidine |
| 157 (E) | 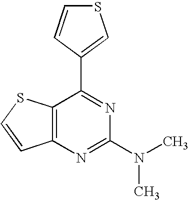 | N,N-dimethyl-4-(3-thienyl)thieno[3,2-d]pyrimidine- 2-amine |
| 158 (B) | 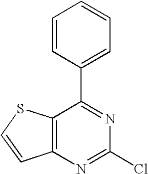 | 2-chloro-4-phenylthieno[3,2-d]pyrimidine |
| 159 (E) | 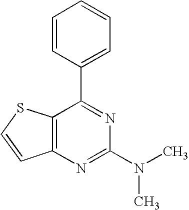 | N,N-dimethyl-4-phenylthieno[3,2-d]pyrimidine-2- amine |
| 160 (B) | 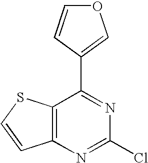 | 2-chloro-4-(3-furyl)thieno[3,2-d]pyrimidine |
| 161 (E) | 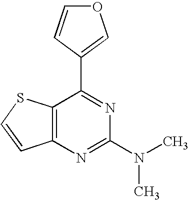 | N,N-dimethyl-4-(3-furyl)thieno[3,2-d]pyrimidine-2- amine |
| 162 (A) | 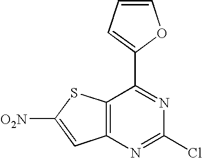 | 2-chloro-4-(2-furyl)-6-nitrothieno[3,2-d]pyrimidine |
| 163 (B) | 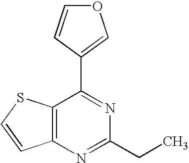 | 2-ethyl-4-(3-furyl)thieno[3,2-d]pyrimidine |
| 164 (B) | 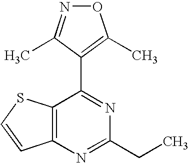 | 4-(3,5-dimethyl-4-isoxazolyl)-2-ethylthieno[3,2- d]pyrimidine |
| 165 (B) | 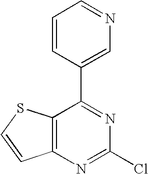 | 2-chloro-4-(3-pyridyl)thieno[3,2-d]pyrimidine |
| 166 (E) | 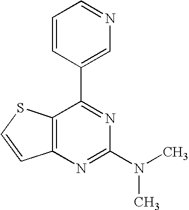 | N,N-dimethyl-4-(3-pyridyl)thieno[3,2-d]pyrimidine- 2-amine |
| 167 (C) | 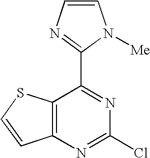 | 2-chloro-4-(1-methyl-1H-imidazol-2-yl)thieno[3,2- d]pyrimidine |
| 168 (E) | 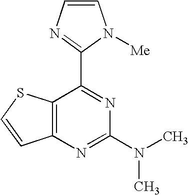 | N,N-dimethyl-4-(1-methyl-1H-imidazol-2- yl)thieno[3,2-d]pyrimidine-2-amine |
| 169 (E) | 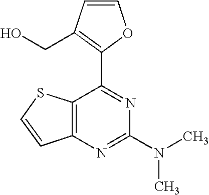 | N,N-dimethyl-4-(3-hydroxymethyl-2- furyl)thieno[3,2-d]pyrimidine-2-amine |
| 170 (E) | 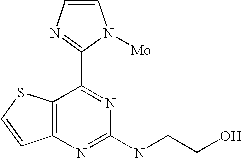 | N-(2-hydroxyethyl)-4-(1-methyl-1H-imidazol-2- yl)thieno[3,2-d]pyrimidine-2-amine |
| 171 (E) | 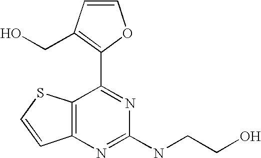 | N-(2-hydroxyethyl)-4-(3-hydroxymethyl-2- furyl)thieno[3,2-d]pyrimidine-2-amine |
| 172 (C) | 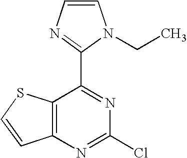 | 2-chloro-4-(1-ethyl-1H-imidazol-2-yl)thieno[3,2- d]pyrimidine |
| 173 (E) | 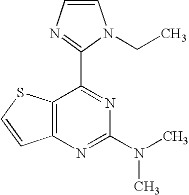 | N,N-dimethyl-4-(1-ethyl-1H-imidazol-2- yl)thieno[3,2-d]pyrimidine-2-amine |
| 174 (E) | 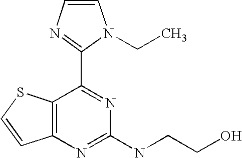 | 4-(1-ethyl-1H-imidazol-2-yl)-N-(2- hydroxyethyl)thieno[3,2-d]pyrimidine-2-amine |
| 175 (C) | 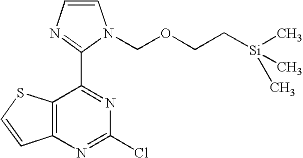 | 2-chloro-4-(1-(2-trimethylsilylethoxymethyl)-1H- imidazol-2-yl)thieno[3,2-d]pyrimidine |
| 176 (E) | 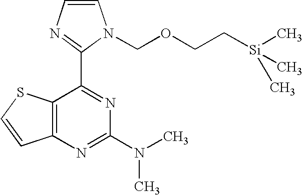 | N,N-dimethyl-4-(1-(2-trimethylsilylethoxymethyl)- 1H-imidazol-2-yl)thieno[3,2-d]pyrimidine-2-amine |
| 177 (C) | 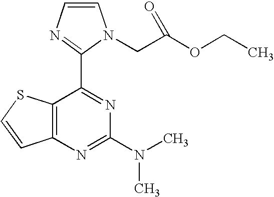 | N,N-dimethyl-4-((1-ethoxycarbonylmethyl)-1H- imidazol-2-yl)thieno[3,2-d]pyrimidine-2-amine |
| 178 (K) | 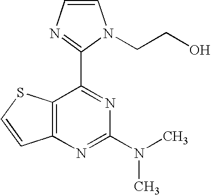 | N,N-dimethyl-4-(1-(2-hydroxyethyl)-1H-imidazol-2- yl)thieno[3,2-d]pyrimidine-2-amine |
| 179 (C) | 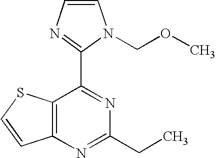 | 2-ethyl-4-(1-methoxymethyl-1H-imidazol-2- yl)thieno[3,2-d]pyrimidine |
| 180 (C) | 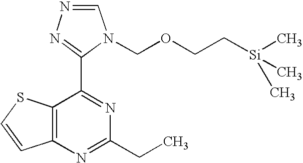 | 2-ethyl-4-(4-(2-trimethylsilylethoxymethyl)-4H- 1,2,4-triazol-3-yl)thieno[3,2-d]pyrimidine |
| 181 (C) | 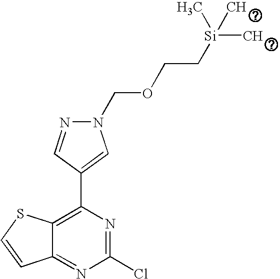 | 2-chloro-4-(1-(2-trimethylsilylethoxymethyl)-1H- pyrazol-4-yl)thieno[3,2-d]pyrimidine |
| 182 (C) | 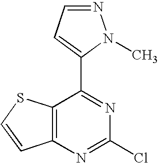 | 2-chloro-4-(1-methyl-1H-pyrazol-5-yl)thieno[3,2- d]pyrimidine |
| 183 (E) | 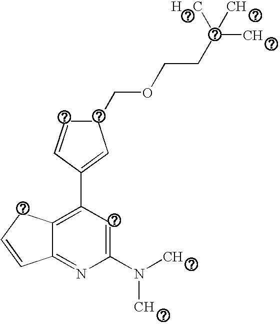 | N,N-dimethyl-4-(1-(2-trimethylsilylethoxymethyl)- 1H-pyrazol-4-yl)thieno[3,2-d]pyrimidine-2-amine |
| 184 (E) | 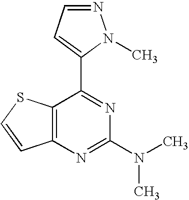 | N,N-dimethyl-4-(1-methyl-1H-pyrazol-5- yl)thieno[3,2-d]pyrimidine-2-amine |
| 185 (D) | 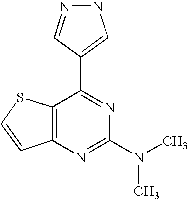 | N,N-dimethyl-4-(1H-pyrazol-4-yl)thieno[3,2- d]pyrimidine-2-amine |
| 186 (C) | 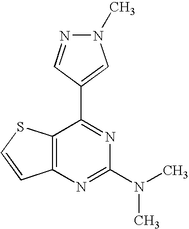 | N,N-dimethyl-4-(1-methyl-1H-pyrazol-4- yl)thieno[3,2-d]pyrimidine-2-amine |
| 187 (C) | 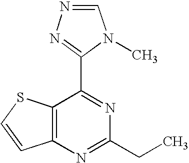 | 2-ethyl-4-(4-methyl-4H-1,2,4-triazol-3- yl)thieno[3,2-d]pyrimidine |
| 188 (A) | 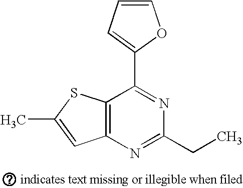 | 2-ethyl-4-(2-furyl)-6-methylthieno[3,2-d]pyrimidine |

An additional preferred A2AR antagonist molecule is set out in WO/2004/058139. KW-6002 (istradefylline) is (E)-8-(3,4-dimethoxystyryl)-1,3-diethyl-7-methylxanthine. KW-6002 has been evaluated humans as a treatment for Parkinson's disease (W. Bara-Jimenez, et al, Adenosine A_{2A} receptor antagonist treatment of Parkinson's disease. Neurology. 2003 Aug. 12; 61(3):293-6). Istradefylline and related A2AR antagonists are disclosed in WO 99/12546 and some examples are shown below.

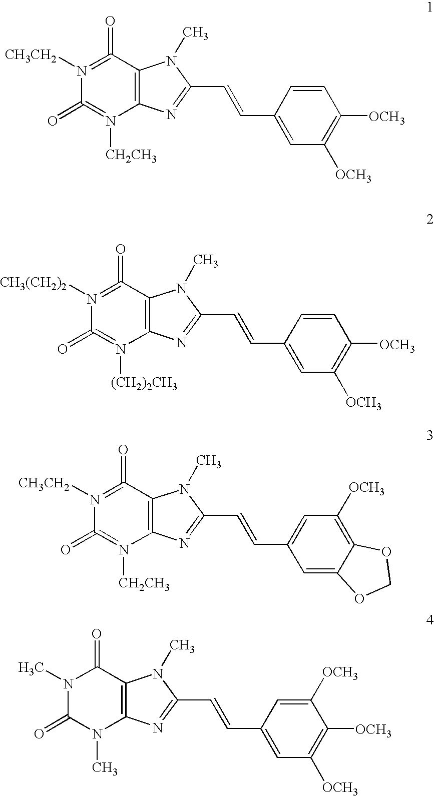

A broader class of istradefylline-related A2AR antagonists is represented by formula (II):

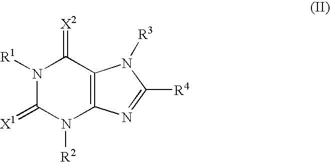

wherein R¹, R² and R³ independently represent hydrogen, lower alkyl, lower alkenyl or lower alkynyl; R⁴ represents cycloalkyl, —(CH₂)ₙ—R⁵ (wherein R⁵ represents substituted or unsubstituted aryl, or a substituted or unsubstituted heterocyclic group, and n is an integer of 0 to 4), or the following group:

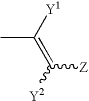

wherein Y¹ and Y² independently represent hydrogen, halogen or lower alkyl, and Z represents substituted or unsubstituted aryl, the following group:

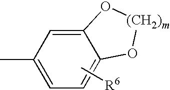

wherein R⁶ represents hydrogen, hydroxy, lower alkyl, lower alkoxy, halogen, nitro or amino, and m is an integer of 1 to 3, or a substituted or unsubstituted heterocyclic group; and X¹ and X² independently represent O or S, or pharmaceutically acceptable salts thereof.

Additional A2AR antagonists of this nature are described in detail in US. Patent Application No. 2006/0178379 and are listed below:

In certain embodiments, the A2A receptor antagonist is represented by formula (II-A):

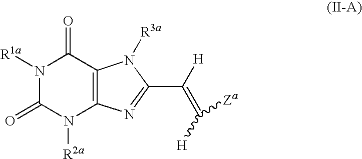

wherein R^{1a} and R^{2a} represent independently methyl or ethyl; R^{3a} represents hydrogen or lower alkyl; and Z^{a} represents

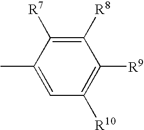

(in which at least one of R⁷, R⁸ and R⁹ represents lower alkyl or lower alkoxy and the others represent hydrogen; R¹⁰ represents hydrogen or lower alkyl) or

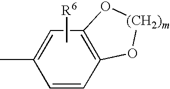

(in which R⁶ and m have the same meanings as defined above, respectively);

and pharmaceutically acceptable salts thereof.

In certain aspects, the A_{2A} receptor antagonist is represented by formula (II-B):

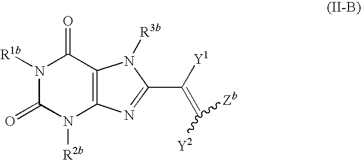

wherein R^{1b} and R^{2b} represent independently hydrogen, propyl, butyl, lower alkenyl or lower alkynyl; R^{3b} represents hydrogen or lower alkyl; Z^{b} represents substituted or unsubstituted naphthyl, or

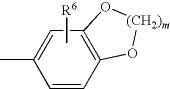

(in which R⁶ and m have the same meanings as defined above); and Y¹ and Y² have the same meanings as defined above, respectively;

and pharmaceutically acceptable salts thereof.

In other embodiments the A_{2A} receptor antagonist is represented by formula (II-C):

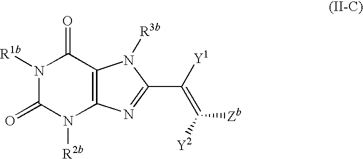

wherein R^{1b} and R^{2b} represent independently hydrogen, propyl, butyl, lower alkenyl or lower alkynyl; R^{3b} represents hydrogen or lower alkyl; Z^{b} represents substituted or unsubstituted naphthyl, or

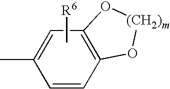

(in which R⁶ and m have the same meanings as defined above, respectively); and Y¹ and Y² have the same meanings as defined above, respectively;

and pharmaceutically acceptable salts thereof.

The adenosine A_{2A} receptor antagonist used in the disclosed methods is not limited as long as it has A_{2A} receptor antagonistic activity. Examples thereof include compounds disclosed in U.S. Pat. No. 5,484,920, U.S. Pat. No. 5,703,085, WO 92/06976, WO 94/01114, U.S. Pat. No. 5,565,460, WO 98/42711, WO 00/17201, WO 99/43678, WO 01/92264, WO 99/35147, WO 00/13682, WO 00/13681, WO 00/69464, WO 01/40230, WO 01/02409, WO 01/02400, EP 1054012, WO 01/62233, WO 01/17999, WO 01/80893, WO 02/14282, WO 01/97786, and the like.

The pharmaceutically acceptable acid addition salts of istradefylline include inorganic acid addition salts such as hydrochloride, sulfate and phosphate, and organic acid addition salts such as acetate, maleate, fumarate, tartrate, citrate and methanesulfonate; the pharmaceutically acceptable metal salts include alkali metal salts such as sodium salt and potassium salt, alkaline earth metal salts such as magnesium salt and calcium salt, aluminum salt, and zinc salt; the pharmaceutically acceptable ammonium salts include ammonium and tetramethylammonium; the pharmaceutically acceptable organic amine addition salts include salts with morpholine and piperidin; and the pharmaceutically acceptable amino acid addition salts include salts with lysine, glycine and phenylalanine.

Istradefylline can be produced by the method disclosed in Japanese Published Unexamined Patent Application No. 211856/94, Japanese Published Unexamined Patent Application No. 16559/94 or WO 94/01114, or according to these methods. The desired compound in the process can be isolated and purified by purification methods conventionally used in synthetic organic chemistry, such as filtration, extraction, washing, drying, concentration, recrystallization or various kinds of chromatography

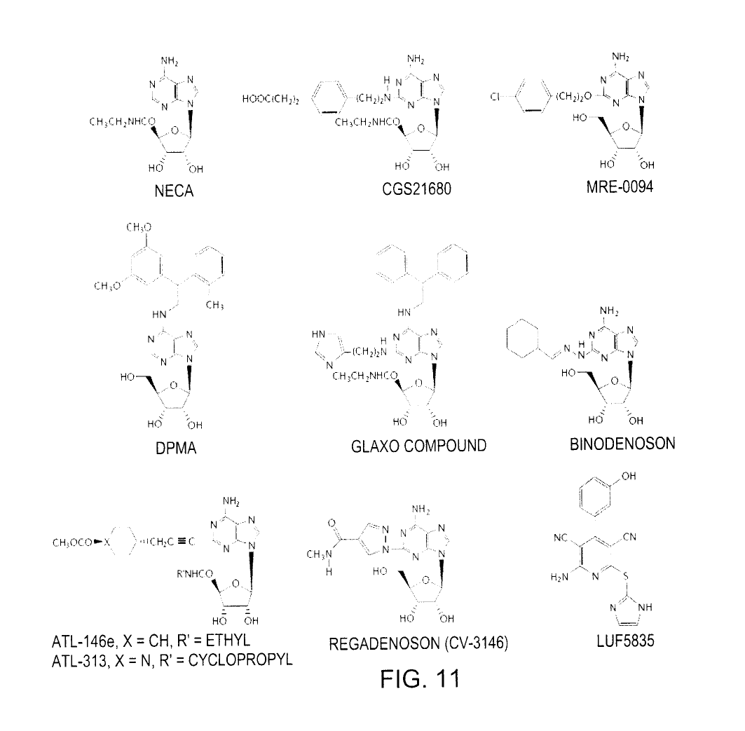

Additional A2AR antagonists are depicted in FIG. 11.

The adenosine receptor inhibitor may also be an antisense molecule or catalytic nucleic acid molecule (e.g., a ribozyme) that specifically binds mRNA encoding an adenosine receptor, e.g., encoding an A2a or A2b adenosine receptor. The antisense molecule or catalytic nucleic acid molecule can be based on an adenosine receptor locus, e.g., the adenosine receptor A2a or A2b locus (e.g., GenBank accession numbers AH003248 and NM000676, respectively). An antisense construct includes the reverse complement of at least part of the adenosine receptor cDNA coding sequence, the adenosine receptor cDNA or gene sequence or flanking regions thereof. The antisense molecule or catalytic nucleic acid may alternatively target biochemical pathways downstream of the adenosine receptor. For example, the antisense molecule or catalytic nucleic acid can inhibit an enzyme involved in the Gs protein-dependent intracellular pathway, e.g., adenylyl cyclase.

The introduced sequence need not be the full-length human adenosine receptor cDNA or gene or reverse complement thereof, and need not be exactly homologous to the equivalent sequence found in the cell type to be transformed. Antisense molecules can be made using known techniques in the art (see, e.g., Agrawal, Methods in Molecular Biology, Humana Press Inc., 1993, Vol. 20 (“Protocols for Oligonucleotides and Analogs”)).

The antisense molecule may be conjugated to another molecule, e.g., a peptide, hybridization triggered cross-linking agent, transport agent, or hybridization-triggered cleavage agent. A targeting moiety can also be included that enhances uptake of the molecule by cells, e.g., tumor cells. The targeting moiety can be a specific binding molecule, such as an antibody or fragment thereof that recognizes a molecule present on the surface of the cell, e.g., tumor cell.

Alternatively, the therapeutic agent is a catalytic nucleic acid, such as a ribozyme (a synthetic RNA molecule that possesses highly specific endoribonuclease activity). The production and use of ribozymes are disclosed in, e.g., U.S. Pat. Nos. 4,987,071 and 5,543,508. Ribozymes can be synthesized and administered to a cell or a subject, or can be encoded on an expression vector, from which the ribozyme is synthesized in the targeted cell (see, e.g., PCT publication WO 9523225, and Beigelman et al., Nucl. Acids Res. 23:4434-42, 1995). Examples of oligonucleotides with catalytic activity are described in, e.g., PCT Publication Nos. WO 9506764 and WO 9011364, and Sarver et al., Science 247:1222-1225, 1990. The inclusion of ribozyme sequences within antisense RNAs can be used to confer RNA cleaving activity on the antisense RNA, such that endogenous mRNA molecules that bind to the antisense RNA are cleaved, which, in turn, leads to an enhanced antisense inhibition of endogenous gene expression.

In other embodiments, the therapeutic agent is an adenosine receptor agonist, e.g., an A1 or A3 adenosine receptor agonist, or any other Gi-protein linked adenosine receptor agonist. Exemplary agonists include, without limitation, N6-Cyclopentyladenosine (CPA), 2-chloro-N(6)-methyl-4′-thioadenosine-5′-methyluronamide, and agonists described in Jeong et al., J. Med. Chem. 49:273-81, 2006, and in U.S. Pat. No. 6,586,413.

In other situations, the therapeutic agent may be an agent that decreases the local tissue accumulation of extracellular adenosine. The agent may render extracellular adenosine non-functional (or decrease such function), such as an agent that modifies the structure of adenosine to nullify the ability of adenosine to signal through adenosine receptors. Such agents can be, e.g., an enzyme (e.g., adenosine deaminase (ADA)) or another catalytic molecule that selectively binds and destroys the adenosine, thereby abolishing or significantly decreasing the ability of endogenously formed adenosine to signal through adenosine receptors. The therapeutic agent may be, e.g., polyethylene glycol-modified adenosine deaminase (ADA-PEG), such as ADAGEN™ (Enzon Pharmaceuticals, Inc., Bridgewater, N.J.). Alternatively, the therapeutic agent may inhibit extracellular adenosine by preventing or decreasing formation of extracellular adenosine, and/or preventing or decreasing the accumulation of extracellular adenosine. For example the therapeutic agent may be an inhibitor of CD39 ecto-apyrase (ADPase/ATPase) and/or 5′-ecto-nucleotidase (CD73) (see, e.g., Eltzschig et al., Methods Mol. Biol. 341:73-87, 2006).

In other embodiments, the subject method can be practiced through the administration of a vaccine in conjunction with an HIF-1α antagonist.

The therapeutic agent may also be one or more of an immunosuppressive, an immunostimulant (e.g., IFA, a COX-2 inhibitor, IL-12, N-acetyl-cysteine, or a saponin, e.g., QS-23), an anti-cancer agent, an anti-inflammatory, an anti-infective, a vaccine, an agent that decreases inflammation-associated local tissue hypoxia, or an agent that decreases the redox status of molecules in an inflamed local tissue environment. In some embodiments, the therapeutic agent is AS-101 (Wyeth-Ayerst Labs., Philadelphia, Pa.), bropirimine (Upjohn, Kalamazoo, Mich.), gamma interferon (Genentech, San Francisco, Calif.), GM-CSF (Genetics Institute, Cambridge, Mass.), IL-2 (Cetus, Emeryville, Calif. or Hoffman-LaRoche, Nutley, N.J.), human immune globulin (Cutter Biological, Berkely, Calif.), IMREG (Imreg, New Orleans, La.), SK&F 106528 (Genentech, San Francisco, Calif.), TNF (Genentech, San Francisco, Calif.), azathioprine (such as Azasan™ by Salix, Raleigh, N.C., or Imuran™ by GlaxoSmithKline, Research Triangle Park, NC), cyclophosphamide (e.g., Cytoxan™ by Bristol-Myers Squibb, Evansville, Ind.), chlorambucil (e.g., Leukeran™ by GlaxoSmithKline, Research Triangle Park, NC), or methotrexate (Ben Venue Laboratories, Bedford, Ohio). The therapeutic agent may also be chemotherapeutic compound, such as ifosfamide (e.g., Ifex™ by Bristol-Myers Squibb, Evansville, Ind.), cisplatin (e.g., Platinol™ by Bristol Myers-Squibb, Princeton, N.J.), procarbazine (e.g., Matulane™ by Sigma Tau Pharms, Gaithersburg, Md.), etoposide (e.g., VePesid™ by Bristol-Myers Squibb, Evansville, Ind.), carmustine (e.g., BiCNU™ by Bristol-Myers Squibb, Evansville, Ind.), vincristine (e.g., Oncovin™ by Gensia Sicor Pharmaceuticals, Inc. Irvine, Calif.), vinblastine (e.g., Velbe™ by Eli Lilly and Co, Indianapolis, Ind.), gencitabine (e.g., Gemzar™ by Eli Lilly, Indianapolis, Ind.), 5-fluorouracil (Alfa Chem, Kings Point, N.Y.), paclitaxel (e.g., Taxol™ by Bristol-Myers Squibb, Evansville, Ind.), or doxorubicin (e.g., Doxil™ by Ortho Biotech Products, Bridgewater, N.J.).

Alternatively, the therapeutic agent may be an anti-viral immune cell, such as one produced by incubating immune cells under hypoxic culture conditions, thereby producing an immune cell that is resistant to hypoxia-produced extracellular adenosine. As used herein, the term “anti-viral immune cell” means a T cell that can recognize and be activated by a viral peptide expressed on the surface of a virus-infected cell. Such immune cells include cytotoxic T lymphocytes (CTL) or a lymphokine-activated killer (LAK) cells. The cells can be produced by culturing peripheral blood cells from a subject in hypoxic culture conditions comprising less than 4% oxygen, between 0.5% and 5% oxygen, between 1% and 4% oxygen, between 1% and 3% oxygen, or between 1% and 2% oxygen. The cells are incubated in the presence of one or more peptides expressed on the surface of virus-infected or cancerous cells (see, e.g., Gattinoni et al., Nat. Rev. Immunol. 6:383-93, 2006).

The therapeutic agent may have an affinity (tropism) for tumor cells, and the oxygen promotes the immune response against the tumor. Without being bound by theory, the therapeutic agent may selectively accumulate in the tumor due to tropism for the tumor cells or the local environment. For example, the therapeutic agent can be delivered to tumors after conjugation with a tumor-recognizing monoclonal antibody (see, e.g., Elbayoumi et al., Eur. Nucl. Med. Mol. Imaging 33:1196-1205, 2006).

Methods such as the Nanocell method (US 2005-0266067 A1, US 2007-0053845 A1) may be used to target therapeutic agents to the tumor. In this method for treating cancer, an antiangiogenic agent is loaded inside the lipid vesicle and is released before the anti-neoplastic/chemotherapeutic agent inside the inner nanoparticle. This results in the collapse of the vasculature feeding the tumor, and also leads to the entrapment of the anti-neoplastic agent-loaded nanocores inside the tumor with no escape route. The anti-neoplastic agent is released slowly resulting in the killing of the nutrient-starved tumor cells. In other words, this double balloon drug delivery system allows one to load up the tumor with an anti-neoplastic agent and then cut off the blood supply to the tumor. This sequential process results in the entrapment of the toxic chemotherapeutic/antineoplastic agent within the tumor, leading to increased and selective toxicity against the tumor cells, and less drug is present in the systemic circulation, since it cannot leak out from the functionally avascular tumor site, resulting in less side effects. This technique also overcomes the hypoxia caveat, as the tumor-entrapped cytotoxic chemotherapeutic cell kills off the tumor cells that would have otherwise survived in the hypoxic growth factor-rich environment resulting from the vascular shutdown.

Sometimes, the therapeutic agent is an immunotoxin that accumulates in the tumor due to its selective interactions with tumor-specific antigens. These therapeutic agents can cause direct destruction of tumor cells, although in some instances, destruction of the tumor can be incomplete. Without being bound by theory, the death of a portion of the tumor cells can create an inflammatory environment within the tumor and activates tumor infiltrating immune cells (macrophages and T cells). One exemplary therapeutic agent is anti-CD19 immunotoxin (IT) (HD37-dgRTA), which is effective in killing B-lineage leukemia cells and in curing severe combined immunodeficient mice with acute lymphoblastic leukemia (see Herrera et al., Leuk. Lymphoma 47:2380-2387, 2006). Other such agents are known in the art.

The therapeutic agent may initiate the anti-tumor process in vivo. For example, when the therapeutic agent is an immune cell activating reagent coupled to a bifunctional antibody that binds a tumor specific antigen and binds a T cell or macrophage-activating ligand, the therapeutic agent can accumulate in the tumor due to its selective interactions with tumor-specific antigens. The therapeutic agent may also direct activation of tumor infiltrating immune cells, which destroys tumor cells. This activation of immune cells and tumor cells death creates an inflammatory environment within the tumor and also activates tumor infiltrating immune cells (e.g., macrophages and T cells).

In other instances, the therapeutic agent is a population of immune cells, such as tumor defense-resistant immune cells, that is specific for tumor antigens. Such tumor defense-resistant immune cells are administered alone or in combination with other ligands that enhance antitumor activity of tumor defense-resistant immune cells (e.g., CTLA4 ligand; Kuhns et al., Proc. Natl. Acad. Sci. USA 97:12711, 2001) or in combination with the removal of CD25⁺ regulatory T cells. Depletion of either of these two immunoregulatory mechanisms improves anti-tumor CTL activity (see Sutmuller et al., J. Exp. Med. 94:823-32, 2001). The tumor defense-resistant immune cells can be prepared, e.g., by incubating them under hypoxic culture conditions, leading to the loss of (or reduction of) adenosine receptors, and thereby rendering these cells uninhabitable by tumor-associated adenosine. The hypoxic culture conditions may comprise less than 4% oxygen, comprise between 0.5% and 5% oxygen, between 1% and 4% oxygen, between 1% and 3% oxygen, or between 1% and 2% oxygen. Alternatively, tumor defense-resistant immune cells can be prepared by incubating them in the presence of adenosine analogs to provide selective negative pressure to prevent or decrease expansion of adenosine receptor-expressing immune cells.

As used herein, “immune cell” means any cell involved in a host defense mechanism, such as cells that produces pro-inflammatory cytokines, and such as cells that participate in tissue damage and/or disease pathogenesis. Examples include, but are not limited to, T cells, B cells, natural killer cells, neutrophils, mast cells, macrophages, antigen-presenting cells, basophils, and eosinophils.

As used herein, the term “tumor defense-resistant immune cell” means an anti-tumor T cell having a reduced level of inhibition of one or more of its activities in a tumor microenvironment. For example, a tumor defense-resistant immune cell can have a reduced level of inhibition by down-regulation of A2A and/or A2B adenosine receptors (see, e.g., Ohta et al., Proc. Natl. Acad. Sci. U.S.A. 103:13132-13137, 2006).

A. Exemplary Adenosine Receptor Antagonists

In certain embodiments, the adenosine pathway antagonist can be an adenosine receptor antagonist, such as an adenosine analog or other small organic molecule, that binds to an adenosine receptor and inhibits (partially or completely) the ability of adenosine to induce a receptor-dependent signal. An “adenosine receptor antagonist” refers to a substance that reduces or blocks activity mediated by an adenosine receptor in response to the cognate ligand of that receptor. The activity of the antagonist can be directly at the receptor, e.g., by blocking the receptor or by altering receptor configuration or activity of the receptor. The activity of the antagonist can also be at other points (e.g. at one or more second messengers, kinases, etc.) in a metabolic pathway that mediates the receptor activity. There are a wide variety of adenosine receptor antagonists from which to chose in the practice of the present methods, including pharmacological agents that impair receptor function, small molecules, antibodies that block the receptor, peptides or proteins that block or inhibit the receptor, small interfering RNA molecules that impair or inhibit transcription of a gene encoding the adenosine receptor, anti-sense RNA that impairs or inhibits the transcription of a gene encoding the adenosine receptor, agents that lead to inhibition, down-regulation, or interference with adenosine receptor activity, and ribozymes with a complementary base pair binding portion that binds to adenosine receptor mRNA and a catalytic portion that cleaves said mRNA.

1. Adenosine A2A Receptor Antagonists.

A number of adenosine A2A receptor antagonists are known to those of skill in the art and can be used individually or in conjunction in the methods described herein. Such antagonists include, but are not limited to caffeine and/or a caffeine derivatives, (−)-R,S)-mefloquine (the active enantiomer of the racemic mixture marketed as Mefloquine™), 3,7-Dimethyl-1-propargylxanthine (DMPX), 3-(3-hydroxypropyl)-7-methyl-8-(m-methoxystyryl)-1-propargylxanthine (MX2 or MSX-2), 3-(3-hydroxypropyl)-8-(3-methoxystyryl)-7-methyl-1-propargylxanthin phosphate disodium salt (MSX-3, a phosphate prodrug of MSX-2), 7-methyl-8-styrylxanthine derivatives, SCH 58261, KW-6002, aminofuryltriazolo-triazinylaminoethylphenol (ZM 241385), and 8-chlorostyrylcaffeine, KF17837, VR2006, istradefylline, the VERNALIS drugs such as VER 6489, VER 6623, VER 6947, VER 7130, VER 7146, VER 7448, VER 7835, VER 8177VER-11135, VER-6409, VER 6440, VER 6489, VER 6623 (also called V2006 and VR2006), VER 6947, VER 7130, VER 7146, VER 7448, VER 7835, VER 8177, pyrazolo[4,3-e]1,2,4-triazolo[1,5-c]pyrimidines, and 5-amino-imidazolo-[4,3-e]-1,2,4-triazolo[1,5-c]pyrimidines, and the like. These adenosine A2A receptor antagonists are intended to be illustrative and not limiting.

Xanthine derivatives, [1,2,4]triazolo[1,5-c]pyrimidine derivatives, [1,2,4]triazolo[1,5-a]pyrimidine derivatives, and the like have also been known to have an adenosine A2A receptor inhibitory action. See, for example, U.S. Pat. No. 5,484,920, U.S. Pat. No. 5,703,085, WO 92/06976, WO 94/01114, U.S. Pat. No. 5,565,460, WO 98/42711, WO 00/17201, WO 99/43678, WO 99/26627, WO 01/92264, WO 99/35147, WO 00/13682, WO 00/13681, WO 00/69464, WO 01/40230, WO 01/02409, WO 01/02400, EP 1054012, WO 01/62233, WO 01/17999, WO 01/80893, WO 02/14282, WO 01/97786, WO 03/032996, WO 03/048163, WO 03/049164 and WO 03/049165.

In certain embodiments, an adenosine receptor inhibitor is an agent that reduces the level of that receptor in a cell. Methods of reducing proteins levels are well known in the art and include the use of antisense nucleic acids, siRNAs, miRNAs, ribozymes, morpholino, PNAs, and the like. Another example of an adenosine receptor inhibitor is a molecule that blocks the activity of the adenosine receptor, including an inhibitory antibody or a small molecule.

In certain embodiments, adenosine A2A receptor antagonists are antagonists that have substantially less effect on the adenosine A1 receptor(s). In certain embodiments, the antagonists show at least 2 fold, preferably at least 5 fold, and more preferably at least 10 fold greater inhibitory activity on the A2A receptor as compared to the adenosine A1 receptor. In certain embodiments, the A2AR antagonist is an A1 agonist. In another, not mutually exclusive embodiment, the A2AR antagonist show at least 2 fold, preferably at least 5 fold, and more preferably at least 10 fold greater inhibitory activity on the A2A receptor as compared to the adenosine A3 receptor and/or the A2B receptor.

In other embodiments, the method can be practiced by combining the vaccine with compounds which are A3 or A1 receptor antagonists. Examples of such compounds are disclosed in U.S. Pat. Nos. 6,326,390; 6,407,236; 6,448,253; 6,358,964; and U.S. Publication Nos. 2003/0 144266 and 2004/0067932; all of which are incorporated herein by reference in their entireties.

2. Exemplary cAMP Antagonists

The term “cAMP antagonist” refers to an agent which decreases the intracellular level of, or cellular response to cAMP, including agents which inhibit adenylate cyclase or activate/potentiate phosphodiesterase. As described in further detail, cAMP antagonists, as the term is used herein, also refers to upstream and downstream effectors of cAMP activity, such as inhibitors of protein kinase A (PKA) or agents that effect G proteins.

As above, the subject cAMP antagonists can be chosen on the basis of their selectivity for cAMP-mediated pathways, such as selectivity in antagonism of cAMP-mediated pathways relative to pathways regulated by other cyclic nucleotides and/or selectivity for particular cAMP-dependent enzymes or even isoforms of those enzymes.

A variety of PKA inhibitors are known in the art to be cAMP inhibitors, including both peptidyl and organic compounds. For instance, the PKA inhibitor can be a 5-isoquinolinesulfonamide, such as represented in the general formula:

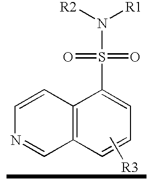

wherein,

R1 and R2 each can independently represent hydrogen, and as valence and stability permit a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH2)m-R8, —(CH2)m-OH, —(CH2)m-O-lower alkyl, —(CH2)m-O-lower alkenyl, —(CH2)n-O—(CH2)m-R8, —(CH2)m-SH, —(CH2)m-S-lower alkyl, —(CH2)m-S-lower alkenyl, —(CH2)n-S—(CH2)m-R8, or

R1 and R2 taken together with N form a heterocycle (substituted or unsubstituted);

R3 is absent or represents one or more substitutions to the isoquinoline ring such as a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH2)m-R8, —(CH2)m-OH, —(CH2)m-O-lower alkyl, —(CH2)m-O-lower alkenyl, —(CH2)n-O—(CH2)m-R8, —(CH2)m-SH, —(CH2)m-S-lower alkyl, —(CH2)m-S-lower alkenyl, —(CH2)n-S—(CH2)m-R8;

R8 represents a substituted or unsubstituted aryl, aralkyl, cycloalkyl, cycloalkenyl, or heterocycle; and

n and m are independently for each occurrence zero or an integer in the range of 1 to 6.

To further illustrate, the PKA inhibitor can be N-[2-((p-bromocinnamyl)amino)ethyl]-5-isoquinolinesulfonamide (H-89; Calbiochem Cat. No. 371963), e.g., having the formula:

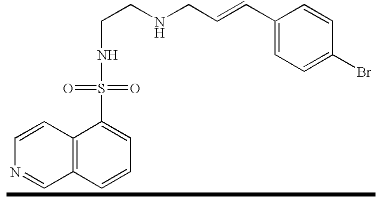

In another embodiment, the PKA inhibitor is 1-(5-isoquinolinesulfonyl)-2-methylpiperazine (H-7; Calbiochem Cat. No. 371955), e.g., having the formula:

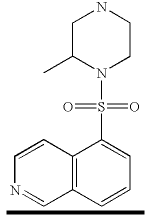

In still other embodiments, the PKA inhibitor is KT5720 (Calbiochem Cat. No. 420315), having the structure

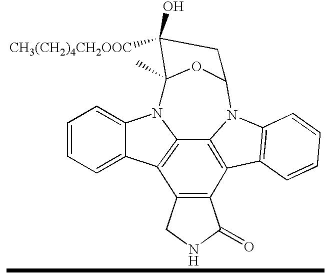

In certain embodiments, a compound which is an agonist or antagonist of PKA is chosen to be selective for PKA over other protein kinases, such as PKC, e.g., the compound modulates the activity of PKA at least an order of magnitude more strongly than it modulates the activity of another protein kinase, preferably at least two orders of magnitude more strongly, even more preferably at least three orders of magnitude more strongly. Thus, for example, a preferred inhibitor of PKA may inhibit PKA activity with a Kᵢ at least an order of magnitude lower than its Kᵢ for inhibition of PKC, preferably at least two orders of magnitude lower, even more preferably at least three orders of magnitude lower. In certain embodiments, the adenosine pathway antagonist inhibits PKC with a Kᵢ greater than 1 μM, greater than 100 nM, preferably greater than 10 nM.

In still other embodiments, the cAMP antagonist is an adenylate cyclase inhibitor.

B. Exemplary HIF-1α Antagonists

In other embodiments, the subject method can be practiced through the administration of a vaccine in conjunction with an HIF-1α antagonist. Exemplary HIF-1α antagonist suitable for use in this version of the methods and compositions described herein include P13 kinase inhibitors; LY294002; rapamycin; histone deacetylase inhibitors such as Depsipeptide FK228 [(E)-(1S,4S,10S,21R)-7-[(Z)-Ethylidene]-4,21-diisopropyl-2-oxa-12,13-dithia-5,8,20,23-tetraazabicyclo[8,7,6]-tricos-16-ene-3,6,9,22-pentanone]; heat shock protein 90 (Hsp90) inhibitors such as geldanamycin, 17-allylamino geldanamycin (17-AAG), and other geldanamycin analogs, radicicol and derivatives thereof such as KF58333; genistein; indanone; staurosporin; protein kinase-1 (MEK1) inhibitors such as PD98059 (2′-amino-3′-methoxyflawne); PX-12 (1-methylpropyl 2 imidazolyl disulfide); PX-478 (S-2-amino-3-[4′-N,N,-bis(2-chloroethyl)amino]phenyl propionic acid N-oxide dihydrochloride); quinoxaline 1,4-dioxides; sodium butyrate (NaB); sodium nitropurruside (SNP) and other NO donors; microtubule inhibitors such as novobiocin, panzem (2-methoxyestradiol or 2-ME2), vincristines, taxanes, epothilones, discodermolide, and derivatives of any of the foregoing; coumarins, barbituric and thiobarbituric acid analogs; camptothecins; and YC-1, a compound described in Biochem. Pharmacol., 2001, 61(8):947-954, incorporated herein by reference, and its derivatives.

In certain embodiments, the HIF-1α inhibitor is a cardiac glycoside. The term “cardiac glycoside” or “cardiac steroid” is used in the medical field to refer to a category of compounds tending to have positive inotropic effects on the heart. As a general class of compounds, cardiac glycosides comprise a steroid core with either a pyrone or butenolide substituent at C17 (the “pyrone form” and “butenolide form”). Additionally, cardiac glycosides may optionally be glycosylated at C3. Most cardiac glycosides include one to four sugars attached to the 3β-OH group. The sugars most commonly used include L-rhamnose, D-glucose, D-digitoxose, D-digitalose, D-digginose, D-sarmentose, L-vallarose, and D-fructose. In general, the sugars affect the pharmacokinetics of a cardiac glycoside with little other effect on biological activity. For this reason, aglycone forms of cardiac glycosides are available and are intended to be encompassed by the term “cardiac glycoside” as used herein. The pharmacokinetics of a cardiac glycoside may be adjusted by adjusting the hydrophobicity of the molecule, with increasing hydrophobicity tending to result in greater absorbtion and an increased half-life. Sugar moieties may be modified with one or more groups, such as an acetyl group.

The cardiac glycoside may comprise a steroid core with either a pyrone substituent at C17 (the “bufadienolides form”), or a butyrolactone substituent at C17 (the “cardenolide” form).

The cardiac glycoside may be selected from: digitoxigenin, digoxin, lanatoside C, Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, convallatoxin, oleandrigenin, bufalin, periplocyrnarin, digoxin (CP 4072), strophanthidin oxime, strophanthidin semicarbazone, strophanthidinic acid lactone acetate, emicyrnarin, sannentoside D, sarverogenin, sarmentoside A, sarmentogenin, or a pharmaceutically acceptable salt, ester, amide, or prodrug thereof.

C. Additional Immunomodulatory Compounds

As used herein and unless otherwise indicated, the terms “immunomodulatory compounds of the invention” and “IMiDs®” (Celgene Corporation) encompass certain small organic molecules that inhibit LPS induced monocyte TNF-α, IL-1β, IL-12, IL-6, MIP-1α, MCP-1, GM-CSF, G-CSF, and COX-2 production. Specific immunomodulatory compounds are discussed below.

TNF-α is an inflammatory cytokine produced by macrophages and monocytes during acute inflammation. TNF-α is responsible for a diverse range of signaling events within cells. Without being limited by a particular theory, one of the biological effects exerted by the immunomodulatory compounds of the invention is the reduction of myeloid cell TNF-α production. Immunomodulatory compounds of the invention may enhance the degradation of TNF-α mRNA.

Further, without being limited by theory, immunomodulatory compounds used in the invention may also be potent co-stimulators of T cells and increase cell proliferation dramatically in a dose dependent manner. Immunomodulatory compounds of the invention may also have a greater co-stimulatory effect on the CD8+ T cell subset than on the CD4+ T cell subset. In addition, the compounds preferably have anti-inflammatory properties against myeloid cell responses, yet efficiently co-stimulate T cells to produce greater amounts of IL-2, IFN-γ, and to enhance T cell proliferation and CD8+ T cell cytotoxic activity. Further, without being limited by a particular theory, immunomodulatory compounds used in the invention may be capable of acting both indirectly through cytokine activation and directly on Natural Killer (“NK”) cells and Natural Killer T (“NKT”) cells, and increase the NK cells' ability to produce beneficial cytokines such as, but not limited to, IFN-γ, and to enhance NK and NKT cell cytotoxic activity.

Specific examples of immunomodulatory compounds include cyano and carboxy derivatives of substituted styrenes such as those disclosed in U.S. Pat. No. 5,929,117; 1-oxo-2-(2,6-dioxo-3-fluoropiperidin-3-yl)isoindolines and 1,3-dioxo-2-(2,6-dioxo-3-fluoropiperidine-3-yl)isoindolines such as those described in U.S. Pat. Nos. 5,874,448 and 5,955,476; the tetra substituted 2-(2,6-dioxopiperidin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), substituted 2-(2,6-dioxopiperidin-3-yl)phthalimides and substituted 2-(2,6-dioxopiperidin-3-yl)-1-oxoisoindoles including, but not limited to, those disclosed in U.S. Pat. Nos. 5,635,517, 6,281,230, 6,316,471, 6,403,613, 6,476,052 and 6,555,554; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds such as those described in U.S. patent publication no. 2003/0045552 published on Mar. 6, 2003, U.S. patent publication no. 2003/0096841 published on May 22, 2003, and International Application No. PCT/US01/50401 (International Publication No. WO 02/059106). The entireties of each of the patents and patent applications identified herein are incorporated herein by reference. Immunomodulatory compounds do not include thalidomide.

Various immunomodulatory compounds of the invention contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of a particular immunomodulatory compounds of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

D. Pharmaceutical Compositions and Methods of Administration.

The therapeutic agents described herein can be formulated as pharmaceutical compositions, e.g., with an appropriate solid or liquid pharmaceutically acceptable carrier or excipient. Such pharmaceutically acceptable carriers and excipients are conventional and known to those of ordinary skill in the art (see, e.g., Harrison's Principles of Internal Medicine, 14th Edition, McGraw-Hill, 1998). For instance, parenteral formulations can include injectable fluids that are pharmaceutically and physiologically acceptable fluid vehicles such as water, physiological saline, other balanced salt solutions, aqueous dextrose, glycerol, or the like. Excipients that can be included are, for instance, other proteins, such as human serum albumin or plasma preparations. If desired, the pharmaceutical composition can also contain minor amounts of non-toxic auxiliary substances, such as wetting or emulsifying agents, preservatives, and pH buffering agents and the like, e.g., sodium acetate or sorbitan monolaurate.

The therapeutic agents described herein can also be formulated using drug carriers to improve, e.g., half-life in vivo, shelf life, bioavailability, or taste. In some situations, the therapeutic agents can be formulated to facilitate application of the therapeutic agent and/or targeted delivery of the therapeutic agent to a specific tissue or to a specific site of pharmacological action. For example, the therapeutic agent can be incorporated into a nanoparticle, a nanoemulsion, a liposome, a prodrug, a polymeric micelle, or a colloidal drug carrier, e.g., as a component of a controlled release drug delivery system (see, e.g., Remington, The Science and Practice of Pharmacology, 20^{th} Edition, Lippincott Williams & Wilkins, 2000).

The dosage form of the pharmaceutical composition will be determined by the mode of administration chosen. For instance, in addition to injectable fluids, topical and oral formulations can be employed. Topical preparations can include eye drops, ointments, sprays, and the like. Oral formulations can be liquid (e.g., syrups, solutions, or suspensions), or solid (e.g., powders, pills, tablets, or capsules). For solid compositions, conventional non-toxic solid carriers can include pharmaceutical grades of mannitol, lactose, starch, or magnesium stearate. Actual methods of preparing such dosage forms are known, or will be apparent, to those of ordinary skill in the art.

The pharmaceutical compositions can be formulated in unit dosage form, suitable for individual administration of precise dosages. For example, one possible unit dosage can contain from about 1 mg to about 1 g of a therapeutic agent described herein. The amount of active compound(s) (i.e., therapeutic agent(s)) administered will be dependent on the specific therapeutic agent(s), the subject being treated, the severity of the affliction, and the manner of administration, and is best left to the judgment of the prescribing clinician. Within these bounds, the pharmaceutical composition to be administered will contain a quantity of the active compounds(s) (i.e., therapeutic agent(s)) in amounts effective to achieve the desired effect in the subject being treated.

The therapeutic agents described herein can be administered to humans or other animals in various manners know to those with skill in the art, e.g., topically, orally, intravenously, intramuscularly, intraperitoneally, intranasally, transdermally, intradermally, intrathecally, and subcutaneously (see, e.g., Harrison's Principles of Internal Medicine, 14th Edition, McGraw-Hill, 1998). The particular mode of administration and the dosage regimen can be selected by an attending physician, taking into account the particulars of the case (e.g., the subject, the disease, the disease state involved, and whether the treatment is prophylactic). Treatment can involve daily or multi-daily doses of therapeutic agents over a period of a few days to months, or even years. In some embodiments, site-specific administration of a therapeutic agent described herein can be used, for instance, by applying a therapeutic agent to a precancerous region, a region of tissue from which a neoplasm has been removed, or a region suspected of being prone to neoplastic development.

In solid dosage forms for oral administration (capsules, tablets, pills, dragees, powders, granules and the like), the subject composition is mixed with one or more pharmaceutically acceptable carriers and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using lactose or milk sugars, as well as high molecular weight polyethylene glycols and the like.

A therapeutically effective amount of a therapeutic agent can be the amount of therapeutic agent necessary to stimulate the immune system of a subject. Specific immunostimulatory effects that can be caused by therapeutic agents are described herein. For example, a therapeutically effective amount of a therapeutic agent can be the amount of therapeutic agent necessary to stimulate an increase in the level of a pro-inflammatory cytokine described herein, e.g., in the blood or urine of a subject. The level of one or more cytokines in the blood or urine from a subject may be measured by ELISA or PCR-based assays or in biological assays. In some instances, an immunostimulatory amount of the therapeutic agent is an amount sufficient to stimulate an immune response (e.g., cause an increase in the level of a cytokine) without causing a substantial cytotoxic effect (e.g., without killing more than 10% of cells in a sample).

As used herein, “administered in combination” means that two or more therapies or agents are administered to a subject at the same time or within an interval, such that there is overlap of an effect of each therapy and/or agent on the subject. The administration of the first and second therapy or agent may be spaced sufficiently close together such that a combinatorial effect is achieved. The interval can be an interval of hr., days, or weeks. The administration of at least one of the therapies or agents, e.g., the first therapy or agent, may be made while the other therapy or agent, e.g., the second therapy or agent, is still present at a therapeutic level in the subject. A “combinatorial therapeutic effect” is an effect, e.g., an improvement, that is greater than one produced by either therapy or agent alone. The difference between the combinatorial therapeutic effect and the effect of each therapy or agent alone can be a statistically significant difference.

### ILLUSTRATIVE EXAMPLES

### Example 1

### Combined Treatment with Caffeine and High Oxygen Improves Rejection of RMA T Lymphoma

1. Methods

Wild type C57BL/6 mice were inoculated with either a high dose of RMA T lymphoma cells (3×10⁵ cells) or a low dose of RMA cells (2×10⁵ cells). The RMA T lymphoma cells express H-2K^{b} molecules. Tumor cells were washed and suspended in PBS and injected s.c. (100 μl of cell suspension/mouse). Perpendicular tumor diameters were measured and tumor volumes were calculated according to the formula a²×b×0.52, where “a” is the smaller and “b” is the larger tumor diameter. The experiment was terminated when tumors reached 2.0 cm in diameter or became ulcerated. Animal experiments were performed according to the protocol approved by Institutional Animal Care and Use Committees (Northeastern University and NIAID).

Treatment with caffeine was started immediately after the inoculation of tumor cells. Caffeine (Sigma, St. Louis, Mo.) was given in the drinking water (0.1% w/v). Control assays of ex vivo serum from caffeine-treated mice confirmed that the in vivo levels of caffeine in serum were sufficiently high to prevent (antagonize) adenosine→A2AR-induced cAMP accumulation in cells.

Treatment with hyperoxia also commenced immediately after inoculation. Hyperoxia treatment was performed by exposing mice to hyperoxic gas (60% oxygen). The mice were placed in an Intensive Care Unit (Thermocare, Incline Village, Nev.), which has enough space to contain mice cages with food, water bottles and lids. These airtight plastic units continuously received a low flow of gas.

2. Results

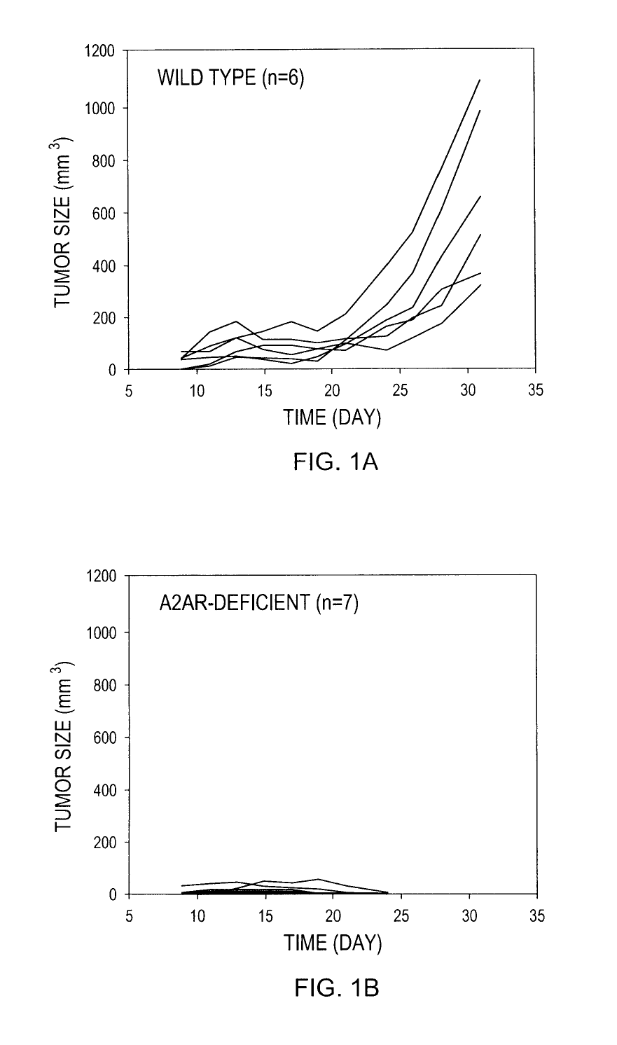

In these studies, conditions of tumor growth in mice were used where the tumor is recognized by the immune system and there is the development of anti-tumor CD8⁺ T cells. However, under these conditions, the tumor defense system prevents anti-tumor T cells from killing the tumor by producing extracellular adenosine in the tumor microenvironment (see Ohta et al., Proc. Natl. Acad. Sci. U.S.A. 103:13132-13137, 2006.) As shown in FIG. 1, A2a adenosine receptor (A2AR) inactivation by genetic mutation led to rejection of established RMA T lymphoma by anti-tumor CD8⁺ T cells. Thus, this result indicates that the A2A adenosine receptor is involved in tumor protection from anti-tumor T cells.

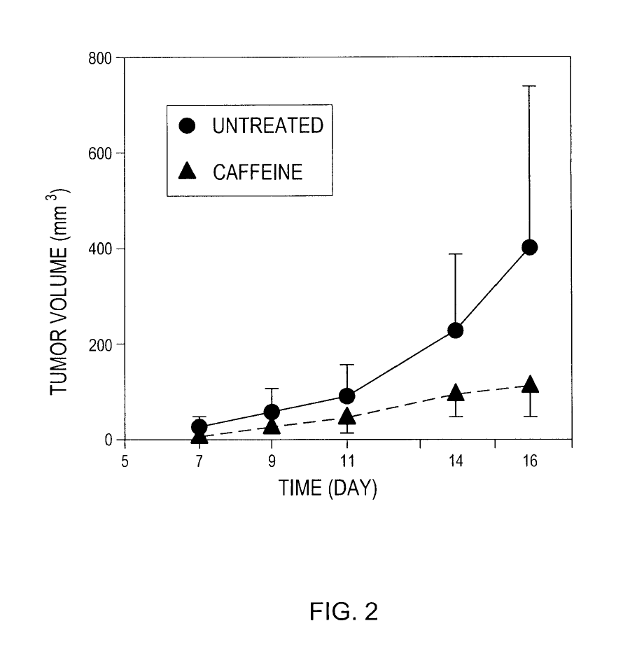

As shown in FIG. 2, treatment with caffeine delayed tumor growth in mice. In spite of significant early inhibition of tumor growth, the tumor eventually became prominent and there was no significant improvement of survival. This shows that while caffeine does facilitate tumor destruction by anti-tumor T cells, it is not sufficient to completely prevent the inhibition by increasing concentrations of tumor-produced extracellular adenosine.

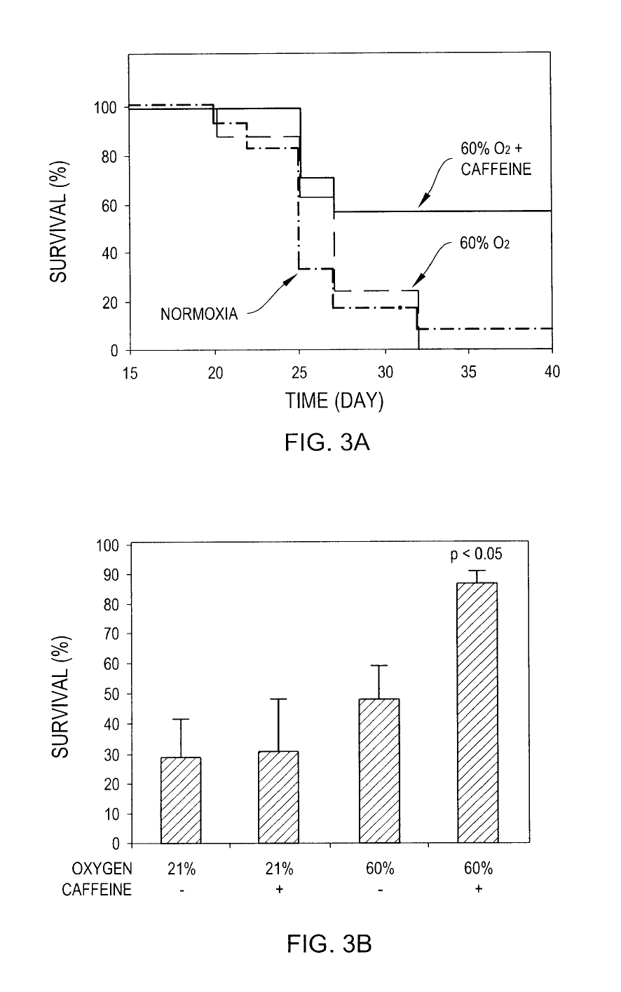

As shown in FIG. 3, combined treatment with caffeine and high oxygen atmosphere significantly improved spontaneous rejection of RMA T lymphoma, ensuring survival of tumor-bearing mice. As shown in FIG. 3A, about 60% of mice survived high dose (3×10⁵ cells, s.c.) RMA challenge by treatment with 60% oxygen plus caffeine (n=14), while most of control (n=12) and 60% oxygen-treated (n=8) mice did not reject the tumor. As shown in FIG. 3B, about 30% of control mice survived from low dose (2×10⁵ cells, s.c.) RMA inoculation. However, combined treatment with caffeine and 60% oxygen significantly improved survival of mice. The results shown here are the average proportion of survival from two independent experiments. The size of group (n) was 6-11.

### Example 2

### Combined Treatment with Caffeine and High Oxygen Improves Effectiveness of Vaccine Immunization

1. Methods

100 μl of 1 mg/ml of a solution of 2,4,6,-Trinitrophenyl hapten conjugated to Keyhole Limpet Hemocyanin (TNP-KLH, Biosearch Technologies Inc., Novato Calif.) with complete Freund's adjuvant (CFA) were injected s.c. to two sites in the back of 3-month old female C57B1/6 mice. Control mice (n=4) were housed in normal oxygen conditions. Other mice (n=8) were kept at 60% oxygen, and half of them were given drinking water containing 1 mg/ml of caffeine instead of regular drinking water. After 14 days mice received booster immunization with TNP-KLH combined with incomplete Freund's adjuvant (IFA). Mice were sacrificed 14 days after booster immunization and blood was collected through heart puncture. Sera were prepared from the blood samples by incubation at room temperature (RT) for 2 hr. and subsequent centrifugation at 2700 g for 3 min. For indirect ELISA measurements of TNP-specific IgM, 96-well flat bottom plates were coated with 2 μg/ml of TNP-BSA at RT overnight, and samples from sera diluted 1:10 were measured using Mouse IgM ELISA Quantitation Kit (Bethyl Labs, Montgomery, Tex.).

2. Results

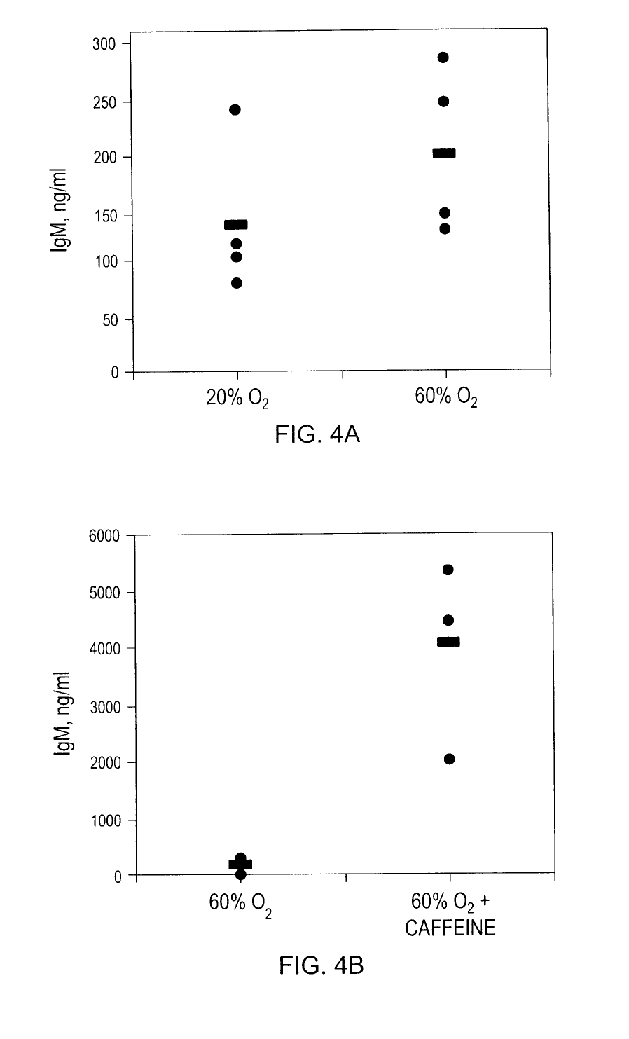

As shown in FIG. 4, combined treatment with caffeine and high oxygen atmosphere significantly improved production of immunoglobulins as measured by indirect ELISA for antigen-specific antibodies. As shown in FIG. 4A, mice that breathed 60% oxygen after vaccination produced more IgM than control immunized mice housed at normal oxygen conditions. Oral administration of caffeine in drinking water further improved effectiveness of immunization, as shown in FIG. 4B.

Those skilled in the art will recognize, or be able to ascertain, using no more than routine experimentation, numerous equivalents to the specific compositions and procedures described herein. Such equivalents are considered to be within the scope of this invention, and are covered by the following claims.

### Example 3

### Tumor Cell Vaccine

1. Model of Human Vaccine in Mice

Tumor melanoma cells are transfected with GM-CSF then irradiated or treated with anti-proliferative drug, mitomycin C, in order to prevent the proliferation of these tumor vaccine cells in a patient to be injected with this vaccine. After injection, the dying cells of cancer vaccine will release GM-CSF into the patient and this results in enhancement of anti-tumor immune response. Hodi F S, Dranoff G. Combinatorial cancer immunotherapy. Adv Immunol. 2006; 90:341-68

2. Method of Treatment with A2AR-Specific Antagonist KW6002 and High Oxygen Atmosphere to Significantly Retard Tumor Growth.

B16 melanoma (1×106 cells, s.c.) were inoculated to syngeneic C57BL/6 mice. All the mice received tumor vaccination, GM-CSF transfectant of B16 cells (1×106 cells, s.c.), for 3 times in every week starting from day 2. On the same day, treatment with KW6002 (2 mg/kg, daily s.c. injection) and/or 60% oxygen was started until the end of the experiment. B16 tumor growth was retarded by treatment with 60% oxygen and combined treatment with KW6002 (left). Perpendicular tumor diameters were measured and tumor volumes were calculated according to the formula a2×b×0.52, where a is the smaller and b is the larger tumor diameter. As shown in FIG. 1, the data represents average tumor size of the same group (n=8). The mice were euthanized when tumor reached 2.0 cm in diameter or became ulcerated. As shown in FIG. 1, in correspondence to the tumor size, the treatment with 60% oxygen and combined treatment with KW6002 prolonged survival of mice (right). The difference was statistically significant with control vs 60% O2 (p=0.033) and control vs 60% O2+KW (p=0.001). The statistics was calculated by log-rank test.

### Example 4

### Protein Vaccine

5-6 weeks old female C57B1/6 mice were immunized by s.c. injections of 0.1 mg of TNP-KLH mixed with CFA. Cages with 5 mice per group of mice were housed in either 20 or 60% oxygen. Indicated groups received 1 mg/ml 1,3,7-trimethylxanthine (TMX, caffeine) in drinking water. After 2 weeks, mice were s.c. injected with 0.1 mg TNP-KLH with IFA and blood was collected 2 weeks after booster immunization. Serums were prepared from the blood samples by incubation at room temperature for 2 hours and subsequent centrifugation at 2700 g for 3 min. For indirect ELISA measurements of TNP-specific IgM, 96-well flat bottom plates were coated with 2 ug/ml of TNP-BSA at room temperature overnight and samples from serums diluted 1:10 were measured using Mouse IgM ELISA Quantitation Kit (Bethyl Labs). As shown in FIG. 2, the A2_{A} antagonist caffeine can enhance the production of specific antibodies of different classes of immunoglobulins.

### Example 5

### The long-Lived A2AR Antagonist KW6002 Dramatically Improves Adoptive Immunotherapy and Enables the Complete T Cell-Mediated Elimination of Lung Metastases

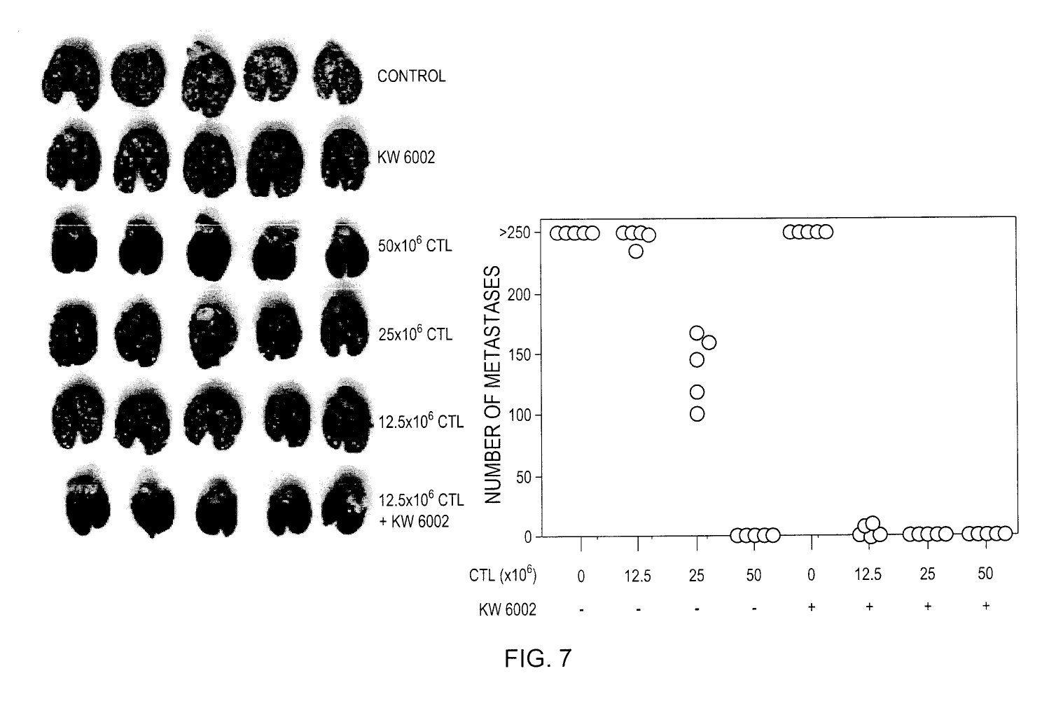

Here, we observed the effect of adoptively transferred anti-tumor T cells on the weakly immunogenic MCA 205 fibrosarcoma lung metastases if treatment included A2AR antagonist KW6002. It is shown that treatment with this long-lived A2AR antagonist may strongly enhance the efficacy of adoptive immunotherapy (FIG. 7). For example, adoptive transfer of 12.5×106 anti-tumor T cells was not effective to prevent tumor metastases, but when co-treated with an A2AR antagonist KW6002, most of the tumor nodules were eliminated.

CTLs were prepared from tumor draining lymph nodes isolated from lymph nodes 12 days after s.c. inoculation with 1.5×105 MCA205 fibrosarcoma. After 2 days anti-CD3 activation and additional 3 days IL-2 expansion, these T cells were injected into mice with 10 days established pulmonary metastases (3×105 MCA205 cells). After the adoptive transfer, the mice received daily i.p. injection of 20 mg/kg of KW6002. The pulmonary metastases were examined by day 21 after tumor inoculation. (Please note that tumors appear as white nodules upon the ink injected lungs.)

### Example 6

### Treatment with Even the Short-Lived A2AR Antagonist 1,3,7-trimethylxanthine (Caffeine) and Exposure of Mice to Hyperoxia (60% Oxygen) Synergistically Enhances Spontaneous Rejection of Tumors

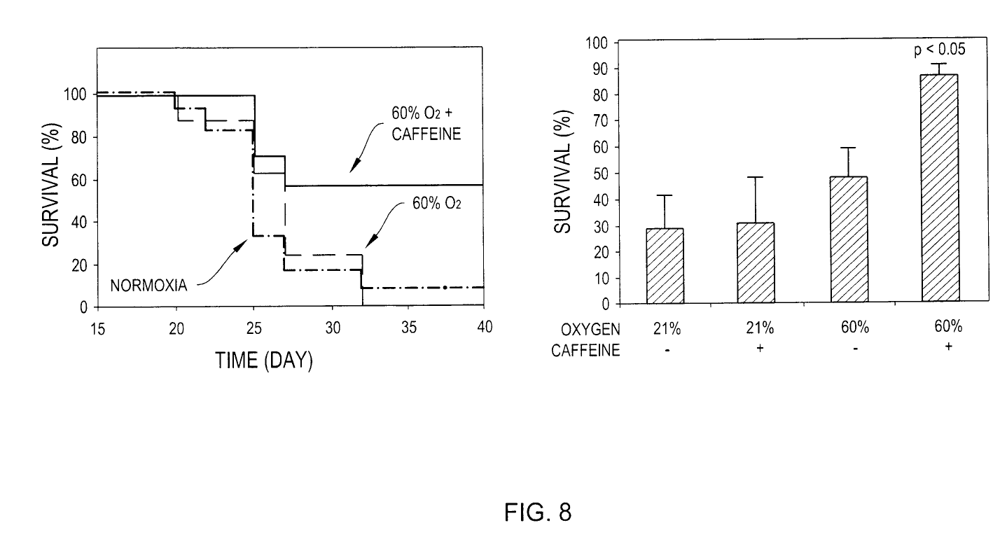

Since it is the tumor hypoxia that is conducive to accumulation of adenosine in TME, we hypothesized that exposure of mice to high oxygen tension (60% O₂) will weaken the tumor tissue hypoxia and decrease the levels of tumor-produced extracellular adenosine. This, in turn, will improve the anti-tumor effects of T cells. Indeed, we demonstrate that high oxygen inhalation and caffeine treatment synergize in preventing the inhibition of anti-tumor T cells thereby dramatically improving tumor rejection and survival (FIG. 8).

FIG. 8 shows that combined treatment with A2AR antagonist (1,3,7 trimethylxanthine, i.e. caffeine) and breathing 60% oxygen significantly improved T cell-mediated rejection of RMA T lymphoma, ensuring survival of tumor-bearing mice. Panel A shows that wild type C57BL/6 mice were inoculated s.c. with high dose (3×105 RMA, s.c.) T lymphoma cells. About 60% of mice survived high dose RMA challenge by treatment with 60% oxygen plus caffeine (n=14), while most of control (n=12) and 60% oxygen-treated (n=8) mice couldn't reject the tumor. Panel B depicts the result when wild type C57BL/6 mice were inoculated s.c. with low dose (2×105 RMA, s.c.) T lymphoma cells About 30% of control mice could survive from low dose RMA inoculation. However, combined treatment with caffeine and 60% oxygen significantly improved survival of mice. The result shown here is average proportion of survival from two independent experiments. The (n) is 6-11 mice.

### Example 7

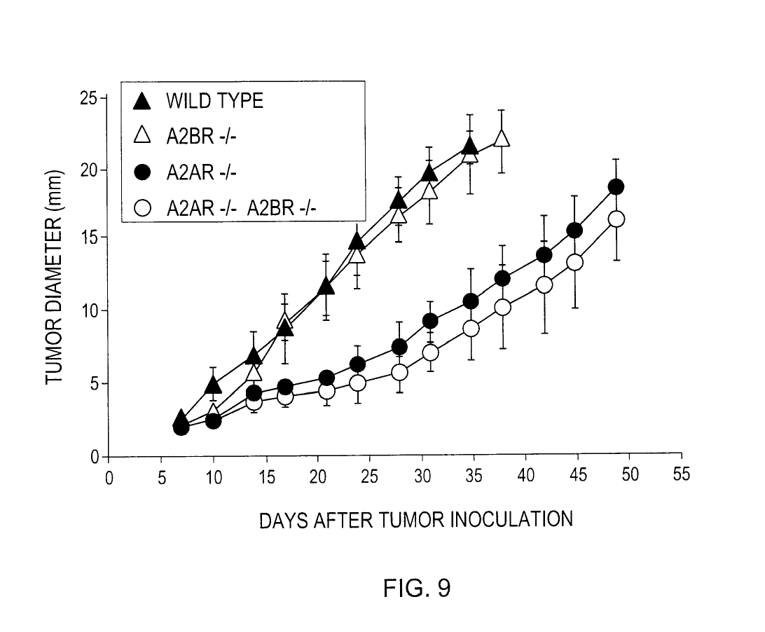

Observations suggesting that it may be sufficient to target only A2AR and not both A2AR and A2BR to accomplish the better rejection of melanoma. These data provided the proof-of-principle for the appealing pharmacological approach where only the A2AR and not the A2BR should be targeted to improve cancer immunotherapy (FIG. 9).

It was important to establish whether low affinity A2BR is as important as A2AR in the inhibition of anti-tumor T cells in TME. This could be done by comparing the tumor rejection in A2AR^{-/-} vs. A2BR^{-/-} vs. A2AR/A2BR double knockout mice. We demonstrate in FIG. 9 that the removal of only A2AR improves the T cell-mediated tumor growth retardation. However, there is no additional improvement in anti-tumor activities in A2AR/A2BR double knockout mice or significant tumor growth retardation in A2BR KO mice.

Deletion of A2BR does not further improve the anti-tumor activity of T cells in A2AR-deleted mice. A2AR or A2BR single knockout mice and A2AR/A2BR double knockout mice were i.d. injected with the weakly immunogenic MCA 205 fibrosarcoma. In A2BR^{-/-} mice, 1×10⁵ MCA 205 tumor cells inoculation led to progressive tumor growth that was not significantly different from control WT mice. In contrast, A2AR^{-/-} mice developed anti-tumor immunity resulting in delay of tumor growth, but all eventually succumbed to the progressive tumor growth. No differences were observed between the A2AR single and A2AR/A2BR double knockout mice indicating that it is the immunosuppressive signaling via A2AR that must be opposed by drugs in order to enhance anti-tumor immunity.

Mice in groups of five were inoculated i.d. with 1×10⁵ MCA 205 tumor cells suspended in 50 μl of HBSS to initiate tumor growth. Tumor sizes were estimated by measuring perpendicular diameters, and the results are expressed as mean diameters of tumors.

These results suggest that A2AR should be targeted in order to improve anti-tumor immunity. Targeting of both A2AR and A2BR does not appear necessary. However, the data suggests that cross-reactivity with A2BR will not reduce the efficacy of an A2AR inhibitor. These are promising observations since less cardiovascular and neurological effects are expected by antagonizing only A2AR than both A2AR/A2BR. Applicants predict that there may be other tumors or different anatomical locations where there will be so much tumor-produced adenosine that even the low affinity A2BR will be triggered to inhibit anti-tumor T cells.

### Example 8

### Expression of Adenosine Receptors in iNKT Cells

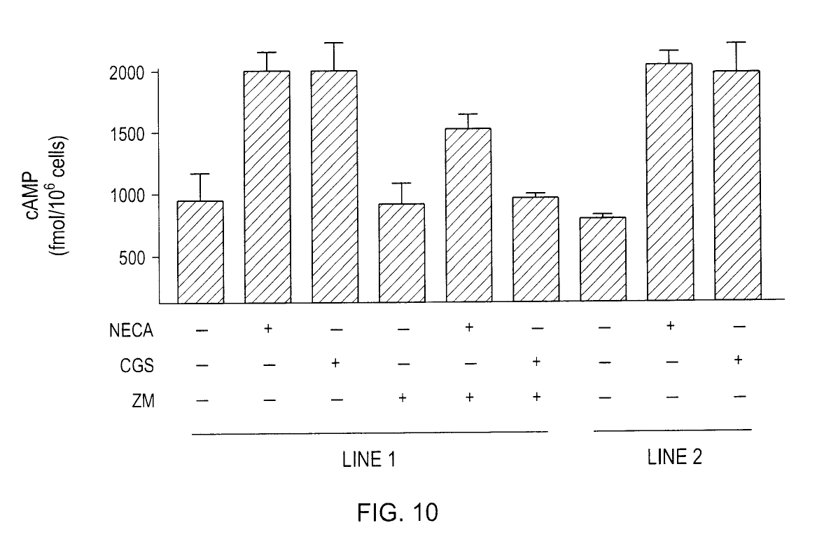

Applicants determined whether human and murine iNKT cells expressed A2A and/or A2B adenosine receptors. Both agonists stimulated comparable levels of cAMP in human iNKT cells, indicating that signaling was predominantly or exclusively through the A2A receptor (FIG. 10) as was the case in mice iNKT cells (not shown). Consistent with these biochemical data, the A2AR, but not A2BR mRNA were detected in human and murine iNKT. We also found that only A2AR was active on both murine and human iNKT in inhibiting IFN-γ and IL-4 secretion. These data support potential role of A2AR in suppressing iNKT cell activity in cancer patients.

Two different cultured iNKT cell lines derived from two healthy donors were stimulated with an agonist selective only for the A2AR(CGS21680) or for both A2AR and A2BR (NECA), and cAMP levels were measured as measure of A2AR vs A2BR functional expression. The activities of both agonists could be blocked by A2AR and A2BR antagonist, ZM241385, confirming the A2AR identification (FIG. 10).

Those skilled in the art will recognize, or be able to ascertain, using no more than routine experimentation, numerous equivalents to the specific substances and procedures described herein. Such equivalents are intended to be encompassed in the scope of the claims that follow the examples below.

Patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.\

1. A method of treating cancer, comprising conjointly administering a therapeutically effective amount of an A2AR antagonist and a cancer vaccine to a patient in need thereof.

2. The method of claim 1, wherein the cancer vaccine is for a solid tumor, and the A2AR antagonist is delivered locally to the site of the tumor.

3. The method of claim 1, wherein the A2AR antagonist is administered repeatedly or continuously for a period of at least 1 month.

4. The method of claim 3, wherein the A2AR antagonist is administered repeatedly or continuously for a period of at least 6 months.

5. The method of claim 1, which includes a first (priming) immunization and at least one subsequent booster immunization, and said A2AR antagonist is administered conjointly with at least one booster immunization.

6. The method of claim 5, wherein the A2AR antagonist is administered continuously or periodically between the priming and booster immunization.

7. The method of claim 1, wherein the vaccine is administered simultaneously with the A2AR antagonist.

8. The method of claim 1, wherein the A2AR antagonist is administered after the vaccine.

9. The method of claim 8, wherein the A2AR antagonist is administered at least 3 days after the vaccine.

10. The method of claim 8, wherein the A2AR antagonist is administered after expansion of T cells specific to the vaccine.

11. The method of claim 8, wherein the A2AR antagonist is administered after the differentiation of CD4+ helper T cells specific to the vaccine.

12. The method of claim 8, wherein the A2AR antagonist is administered at a time when the vaccine or CD4+ helper T cells specific to the vaccine are present at an effective serum concentration.

13. The method of claim 1, wherein the cancer is melanoma, prostate cancer, breast cancer, ovarian cancer, esophageal cancer, or kidney cancer.

14. The method of claim 1, 2, or 5, further comprising conjointly administering oxygen to the patient.

15. The method of claim 14, wherein at least 45% oxygen is administered.

16. The method of claim 15, wherein at least 70% oxygen is administered.

17. The method of claim 16, wherein at least 90% oxygen is administered.

18. The method of claim 14, wherein oxygen is administered conjointly with the A2AR antagonist.

19. The method of claim 1, further comprising conjointly administering at least one additional anti-cancer therapy to the patient, wherein the additional anti-cancer therapy is selected from the group consisting of radiation therapy, chemotherapy, surgery, and vasculature-targeting therapy.

20. The method of claim 1, wherein one or more biomarkers are used to assay the status of the cancer.

21. The method of claim 20, wherein the biomarker is selected from the group consisting of AMACR, PAP, PSM, MAGE, NY-ESO-1, MUM-1, p53, CDK4, HER2/NEU, antigens from Papilloma Virus, antigens from Epstein-Barr Virus, LAGE1, Melan A, MART-1, MAGE-1, MAGE-3, BAGE, GAGE-1, GAGE-2, tyrosinase, gp100, gp75, c-erb-B2, CEA, MUC-1, CA-125, Stn, TAG-72, KSA (17-1A), PSMA, point-mutated RAS, EGF-R, VEGF, GD2, GM2, GD3, Anti-Id, CD20, CD19, CD22, CD36, Aberrant class II, B1, CD25, (IL-2R, anti-TAC), CA-125, CA19-9, PSA, GSTP1, promoter region of GSTP1, NGAL, CD97, CD 55, COX4-2, LAMA2, kallikrein 12, kallikrein 14, kallikrein 15, EPCA, G-CSF, leptin, prolactin, OPN, IGF-II, delta-catenin, ERRγ, hK10, hK6, hK2, alpha-haptoglobin, PKC, calreticulin, 125P5C8, Nicotinamide N-methyltransferase, ULIP proteins, ITGβ6, TIMP-1, Nup88, Csk autoantibodies, VEGFR, Neuropilins, COTA, hnRNP, TSC403, or NCA 50/90.

22. The method of claim 1, wherein the A2AR antagonist is selected from the group consisting of: caffeine and/or a caffeine derivatives; (−)-(R,S)-mefloquine; 3,7-Dimethyl-1-propargylxanthine; 3-(3-hydroxypropyl)-7-methyl-8-(m-methoxystyryl)-1-propargylxanthine; 3-(3-hydroxypropyl)-8-(3-methoxystyryl)-7-methyl-1-propargylxanthine phosphate disodium salt; 7-methyl-8-styrylxanthine derivatives; 7-(2-phenylethyl)-5-amino-2-(2-furyl)-pyrazolo-[4,3-e]-1,2,4-triazolo[1,5c]pyrimidine; (E)-1,3-diethyl-8-(3,4-dimethoxystyryl)-7-methyl-3,7-dihydro-1H-purine-2,6-dione; aminofuryltriazolo-triazinylaminoethylphenol (ZM 241385); 8-chlorostyrylcaffeine; (E)-1,3-dipropyl-8-(3,4-dimethoxystyryl)-7-methyl-3,7-dihydro-1H-purine-2,6-dione; 2-isopropyl-4-(2-thiazolyl)thieno[3,2-d]pyrimidine-2-amine; the VERNALIS drugs such as VER 6489, VER 6623, VER 6947, VER 7130, VER 7146, VER 7448, VER 7835, VER 8177, VER 11135, VER 6409, VER 6440; pyrazolo[4,3-e]1,2,4-triazolo[1,5-c]pyrimidines; and 5-amino-imidazolo-[4,3-e]-1,2,4-triazolo[1,5-c]pyrimidines.

23-24. (canceled)

25. The method of claim 1, further comprising conjointly administering an adjuvant with the cancer vaccine.

26. The method of claim 25, wherein the adjuvant is an aluminum compound or a saponin.

27-34. (canceled)

35. A method of treating solid tumors, comprising conjointly administering a therapeutically effective amount of an A2AR antagonist together with a vasculature-targeting agent to a patient in need thereof.

36-49. (canceled)

50. A method of vaccinating an individual, comprising conjointly administering an effective amount of a pathogen vaccine and an effective amount of an A2AR antagonist to the individual.

51-63. (canceled)

64. A method of vaccinating an individual, comprising:
(a) administering a therapeutically effective amount of a vaccine to an individual,
(b) determining the level of a biomarker in the individual,
(c) determining whether the level of the biomarker is significantly different from a control level, and
(d) only administering an A2AR antagonist to the patient if the biomarker level is significantly different from the control level.

65-75. (canceled)

76. A method of eliciting an enhanced immune response to a cancer cell, comprising conjointly administering a therapeutically effective amount of an A2AR antagonist and a cancer vaccine to a patient in need thereof.

77. A method of eliciting an enhanced immune response to a cancer cell, comprising conjointly administering a therapeutically effective amount of an A2AR antagonist and oxygen to a patient in need thereof.

78. A method of treating a patient, comprising delivering a localized dose of an A2AR antagonist.

79-86. (canceled)

87. A method of enhancing a B cell response of a non-human animal, comprising:
(a) administering an immunogen to a non-human animal, and
(b) conjointly administering an A2AR antagonist to the animal.

88-95. (canceled)

96. A method of screening for an adenosine receptor antagonist, comprising:
(a) contacting an immune cell with an agent;
(b) exposing the immune cell to high oxygen levels, and
(c) assaying for increased activity of the immune cell as compared to a control in the absence of the agent, wherein increased activity of the immune cell indicates that the agent is an adenosine receptor antagonist.

97-99. (canceled)

100. A pharmaceutical preparation comprising an A2AR antagonist and an additional anti-cancer agent.

101. A pharmaceutical preparation comprising an A2AR antagonist and a vasculature-targeting agent.

102. A pharmaceutical preparation comprising an A2AR antagonist and a vaccine.

103. A method of enhancing the immune response of a patient, comprising conjointly administering a therapeutically effective dose of an A2AR antagonist and an inhibitor of an adenosine-producing enzyme.

104-105. (canceled)
